(19) United States
(12) Patent Application Publication
(10) Pub. No.: US 2025/0382631 A1
(43) Pub. Date: Dec. 18, 2025

(54) PROTAC-CID SYSTEMS FOR USE IN MULTIPLEX GENE REGULATION

(71) Applicant: William Marsh Rice University, Houston, TX (US)

(72) Inventors: Xue GAO, Houston, TX (US); Dacheng MA, Houston, TX (US)

(73) Assignee: William Marsh Rice University, Houston, TX (US)

(21) Appl. No.: 18/867,609

(22) PCT Filed: May 19, 2023
(86) PCT No.: PCT/US2023/067256

§ 371 Date: Nov. 20, 2024

Related U.S. Application Data
(60) Provisional application No. 63/344,264, filed on May 20, 2022.

(51) Int. Cl. C12N 15/85; C07K 14/00; C12N 9/00; C12N 9/12; C12N 9/22; C12N 9/78; C12N 15/113; C12N 15/62

(52) U.S. Cl. CPC C12N 15/85; C07K 14/00; C12N 9/1241; C12N 9/226; C12N 9/78; C12N 9/93; C12N 15/113; C12N 15/62; C12Y 603/02; C07K 2319/80; C12N 2310/20

(57) ABSTRACT

The present disclosure provides proteolysis targeting chimeras-based scalable CID (PROTAC-CID) system that repurpose PROTACs for inducible, orthogonal, and multiplex transcriptional activation. When coupled with multi-layer genetic circuits, PROTAC-CID enables digitally inducible DNA manipulations with low basal levels. These PROTAC-CID systems can be delivered in vivo by adeno-associated virus (AAV) to allow ON-OFF genetic switches.

### REFERENCE TO RELATED APPLICATIONS

The present application claims the priority benefit of U.S. provisional application No. 63/344,264, filed May 20, 2022, the entire contents of which are incorporated herein by reference.

### STATEMENT REGARDING FEDERALLY SPONSORED RESEARCH

This invention was made with government support under Grant No. R01 HL157714 awarded by the National Institutes of Health and Grant No. CBET-2143626 awarded by the National Science Foundation. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on May 18, 2023, is named RICEP0108WO_ST26.xml and is 470,213 bytes in size.

### BACKGROUND

### 1. Field

The present disclosure relates generally to the fields of molecular biology and gene regulation. More particularly, it concerns composition and methods that employ proteolysis targeting chimeras to create chemically induced dimeraization systems for transcriptional regulation.

### 2. Description of Related Art

Precise spatiotemporal manipulation of gene expression in living cells is essential for both basic biology research and therapeutic development (1, 2). Inducible and reversible gene expression can overcome safety concerns for gene and cell therapies (3). Small molecule inducers enable precise spatial, temporal, and quantitative gene regulation and have revolutionized biomedical research (3, 4). However, the widely used Tet-On/Off inducible systems derived from bacterial origins can elicit immune responses with leaky baseline gene expressions and the concerns of antibiotic usage in mammals (5, 6). The chemically induced dimerization (CID) system-based inducible gene activation tool is composed of two fusion proteins with small-molecule binding domains fused to a DNA binding domain and a transcriptional activation domain, respectively. In the presence of a small molecule, both fusion proteins bind to the same small molecule, recruit the transactivation domain to DNA, and activate gene expression (2). CID-based gene regulation systems have been used for novel transactivation domain mining (7), CRISPR-based gene activation (8), and tailored antibody N-glycosylation modification (9). In addition to gene regulation, the CID systems have been utilized to regulate protein degradation (10), cell therapy (11), and programmable 3D genome positioning (12, 13).

Most CID systems use naturally existing small molecules from bacteria or plants. Rapamycin is a widely-used CID inducer but with undesirable immunosuppressive effects and autophagy-inducing effects (14, 15). Other CID inducers, such as abscisic acid (ABA) (16) and gibberellic acid analog (GA₃) (17), require high concentrations for efficient protein dimerization. Prior efforts to expand CID toolboxes include designing or mining small molecules (18-21), identifying new protein partners through screening nanobody/antibody libraries (22, 23), or computation-assisted protein design (24, 25). However, the number of highly efficient CIDs remains limited, preventing multiplexing applications in mammalian cells. These requirements and limitations for CID systems have led to the need for a framework that can be used to create a panel of CID systems with multiplex and clinical application potential, such need at present being unfulfilled.

### SUMMARY

To address this unmet need, the inventors repurposed proteolysis targeting chimeras (PROTACs), a rapidly growing group of small molecules that harness the ubiquitin-proteasome system for proximity-induced degradation of the targeted proteins (FIG. 1A) (26, 27). PROTACs are composed of a warhead that binds to the target protein, an anchor ligand that binds to an E3 ubiquitin ligase, and a linker that ties these two parts together (27). At least 1600 PROTACs have been developed, acting on more than 100 human protein targets with multiple E3 ubiquitin ligases (28, 29), mainly for cancer therapy. Herein, PROTACs were repurposed to expand the repertoire for CID-based applications, especially inducible gene regulation.

In one embodiment, provided herein are systems for regulating an inducible protein-protein interaction to execute a biological function, the system comprising: (a) a first fusion protein comprising a domain of interest fused to a first interacting protein; and (b) a second fusion protein comprising a domain of interest fused to a second interacting protein, whereby the presence of a small molecule having a first ligand part capable of binding to the first interacting protein and a second ligand part capable of binding to the second interacting protein induces the protein-protein interaction to execute the biological function.

In some aspects, the biological function is regulating the expression of a first inducible gene, and the system comprises: (a) a first fusion protein comprising a DNA binding domain of a transcription factor fused to a first interacting protein, or a nucleic acid encoding said first fusion protein; (b) a second fusion protein comprising a transcription activator fused to a second interacting protein, or a nucleic acid encoding said second fusion protein; and (c) a nucleic acid comprising an expression cassette wherein the first inducible gene is under the control of a promoter to which the DNA binding domain of the first fusion protein binds, whereby the presence of a small molecule having a first ligand part capable of binding to the first interacting protein and a second ligand part capable of binding to the second interacting protein induces expression of the first inducible gene.

In some aspects, the system further comprises: (d) a third fusion protein comprising a second DNA binding domain of a transcription factor fused to a third interacting protein, or a nucleic acid encoding said third fusion protein; and (e) a fourth fusion protein comprising a second transcription activator fused to a fourth interacting protein, or a nucleic acid encoding said fourth fusion protein; (f) a nucleic acid comprising a second expression cassette comprising a second inducible gene is under the control of a second promoter to which the second DNA binding domain of the third fusion protein binds, whereby the presence of a second small molecule having a third ligand capable of binding to the third interacting protein and a fourth ligand capable of binding to the fourth interacting protein induces expression of the second inducible gene.

In some aspects, the first transcription activator and the second transcription activator are the same. In some aspects, the first DNA binding domain and the second DNA binding domain are different. In some aspects, the first small molecule does not induce expression of the second inducible gene. In some aspects, the second small molecule does not induce expression of the first inducible gene.

In some aspects, the system further comprises: (d) a third fusion protein comprising a second DNA binding domain of a transcription factor fused to a third interacting protein, or a nucleic acid encoding said third fusion protein; and (e) a fourth fusion protein comprising a second transcription activator fused to a fourth interacting protein, or a nucleic acid encoding said fourth fusion protein; wherein the first inducible gene is further under the control of a second promoter to which the second DNA binding domain of the third fusion protein binds, whereby the presence of either (a) a first small molecule having a first ligand capable of binding to the first interacting protein and a second ligand capable of binding to the second interacting protein or (b) a second small molecule having a third ligand capable of binding to the third interacting protein and a fourth ligand capable of binding to the fourth interacting protein induces expression of the first inducible gene.

In some aspects, the first DNA binding domain and the second DNA binding domain are different. In some aspects, the first transcription activator and the second transcription activator are the same. In some aspects, the third interacting protein is the same as the first interacting protein, and the fourth interacting protein is different than the second interacting protein. In some aspects, the third interacting protein is different than the first interacting protein, and the fourth interacting protein is the same as the second interacting protein. In some aspects, the first promoter and the second promoter are the same. In some aspects, the first promoter and the second promoter are different.

In some aspects, the first inducible gene is a first DNA recombinase. In some aspects, the recombinase is Cre recombinase or a Dre recombinase. In some aspects, the system further comprises a nucleic acid comprising a second expression cassette comprising a first gene of interest operably linked to a second promoter, wherein a sequence that prevents expression of the first gene of interest is positioned between the second promoter and the first gene of interest and is flanked by recombinase recognition sequences for the first DNA recombinase. In some aspects, the first gene of interest is a second DNA recombinase, a base editor, a prime editor, or a therapeutic protein. In some aspects, the second promoter is a second inducible promoter. In some aspects, the first inducible promoter and the second inducible promoter are the same.

In some aspects, the system further comprise a nucleic acid comprising a third expression cassette comprising a second gene of interest operably linked to a third promoter, wherein a sequence that prevents expression of the second gene of interest is positioned between the third promoter and the second gene of interest and is flanked by recombinase recognition sequences for the second DNA recombinase. In some aspects, the second gene of interest is a base editor, a prime editor, or a therapeutic protein. In some aspects, the third promoter is a third inducible promoter. In some aspects, the third promoter is a constitutive promoter. In some aspects, the first inducible promoter and the second inducible promoter are the same. In some aspects, the first inducible promoter and the second inducible promoter are different.

In some aspects, the DNA binding domain is a GAL4 DNA binding domain. In some aspects, the transactivation domain is a VP64-p65-Rta (VPR) transactivation domain. In some aspects, the promoter is a GAL4 cognate pUAS promoter or a tetracycline response element.

In some aspects, the biological function is inducing adenine base editing activity, and the system comprises: (a) a first fusion protein comprising an N-terminal portion of an adenine base editor (ABE) deaminase domain fused to a first interacting protein, or a nucleic acid encoding said first fusion protein; (b) a second fusion protein comprising a C-terminal portion of the ABE deaminase domain fused with a CRISPR nuclease and a second interacting protein, or a nucleic acid encoding said second fusion protein; and wherein the presence of a small molecule having a first ligand part capable of binding to the first interacting protein and a second ligand part capable of binding to the second interacting protein induces adenine base editing activity. In some aspects, the CRISPR nuclease is SpCas9 or SpG.

In some aspects, the small molecule is rapamycin. In some aspects, the first or second interaction protein is FRB or FKBP3. In some aspects, each fusion protein comprises two copies of the interacting protein.

In some aspects, the small molecule is a proteolysis targeting chimera (PROTAC). In some aspects, one of the first interacting protein or the second interacting protein is the PROTAC's target protein, and the other of the first interacting protein or the second interacting protein is the PROTAC's E3 ubiquitin ligase. In some aspects, the E3 ubiqutin ligase lacks ubiquitin ligase function. In some aspects, the E3 ubiquitin ligase lacks the seven α-helical bundle domain (HBD). In some aspects, the E3 ubiquitin ligase is unable to interact with Damage Specific DNA Binding Protein 1 (DDB1). In some aspects, the E3 ubiquitin ligase has ubiquitin ligase function. In some aspects, the PROTAC's target protein is a full-length PROTAC target protein. In some aspects, the PROTAC's target protein is the portion of the target protein needed for interaction with the PROTAC. In some aspects, the PROTAC's target protein is the bromodomain of the target protein.

In one embodiment, provided herein are cells comprising the system of any one of the present embodiments. In some aspects, the first inducible gene, the first gene of interest, or the second gene of interest is a site-specific DNA recombinase. In some aspects, the first inducible gene, the first gene of interest, or the second gene of interest is a base editor. In some aspects, the first inducible gene, the first gene of interest, or the second gene of interest is a prime editor. In some aspects, the first inducible gene, the first gene of interest, or the second gene of interest is a therapeutic protein.

In one embodiment, provided herein are methods of inducing site-specific DNA recombination or adenine base editing in a cell, the method comprising contacting the cell of the present embodiments with the first small molecule.

In one embodiment, provided herein are methods of inducing base editing in a cell, the method comprising contacting the cell of the present embodiments with the first small molecule.

In one embodiment, provided herein are methods of inducing prime editing in a cell, the method comprising contacting the cell of the present embodiments with the first small molecule.

In one embodiment, provided herein are methods of expressing a therapeutic protein in a cell, the method comprising contacting the cell of the present embodiment with the first small molecule. In some aspects, the cell is contacting with the first small molecule a second time. In some aspects, the contacting occurs in vivo.

In one embodiment, provided herein are vectors or combinations of vectors comprising the nucleic acids of the system of any one of the present embodiments. In some aspects, the combination of vectors comprises two vectors. In some aspects, the vectors are adeno-associated viral (AAV) vectors. In some aspects, the vectors are optimized for expression in mammalian cells. In some aspects, the vectors are optimized for expression in human cells.

In one embodiment, provided herein are compositions comprising the vector or combination of vectors of any one of the present embodiments. In some aspects, the compositions further comprise a pharmaceutically acceptable carrier.

In one embodiment, provided herein are methods for producing a cell in which a first inducible gene can be inducibly expressed or in which an adenine base editor can be inducibly activated, the method comprising contacting a cell with the composition of any one of the present embodiments, under conditions suitable for expression of the first fusion protein and the second fusion protein. Also provided are cells produced by the methods, where the cells can be plant cells or animal cells, where the cells may be isolated or in an organism.

In one embodiment, provided herein are methods for expressing the first inducible gene or inducing adenine base editing in a cell, the method comprising contacting the cell of the present embodiments with the first small molecule, thereby inducing expression of the first inducible gene or induction of adenine base editing. In some aspects, the cell is in a mammal and the contacting occurs by intravenous or intraperitoneal administration. In some aspects, the expression of the first inducible gene or the adenine base editor treats a disease or disorder in the mammal. In some aspects, the method interferes with RNA splicing of a gene. In some aspects, the interference of RNA splicing of a gene inactivates the expression of the gene.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1E. Repurposing PROTACs for inducible gene activation. (FIG. 1A) Schematic of the PROTAC system for the degradation of target proteins (left). E2, E2 ubiquitin ligase. Ub, ubiquitin. Schematic of the repurposed PROTAC-CID system for inducible gene expression (right). PROTAC target protein or E3 ubiquitin ligase fused with DNA-binding domain GAL4 or transactivation domain VPR. GAL4 binds with the cognate upstream activation sequence promoter (pUAS-1). PROTACs recruit the VPR domain to the pUAS-1 for enhanced yellow fluorescent (EYFP) protein expression. pA, polyA signal. (FIG. 1B) Relative EYFP fluorescence intensity was measured by flow cytometry in response to PROTACs or solvent Dimethylsulfoxide (DMSO) after 2 days of induction. The concentration for each small molecule and the protein fusion strategy are listed in Table 5. The target protein is shown in blue, and E3 ubiquitin ligase is shown in red. (FIGS. 1C-1E) EYFP signal intensity in the presence of 5 μM dTRIM24 (FIG. 1C), 100 nM MZ1 or 1 μM AT1 (FIG. 1D), or 1 μM dBRD9 (FIG. 1E) or DMSO (shown as “−”) with truncated TRIM24, BRD4, and BRD9 as indicated, respectively. Same data is shown in FIG. 1B and FIGS. 1C, 1D, or FIG. 1E with full-length TRIM24, BRD4, and BRD9 for comparison. Data are mean with Standard deviation (SD). n=3 biologically independent samples. AU, arbitrary units. See Methods for EYFP intensity normalization calculation. (FIGS. 1B-1E). HEK293T cells transfected with the reporter plasmid encoding pUAS-1-driven EYFP, with no PROTAC or DMSO added, as the blank control group (Ctrl).

FIGS. 2A-2E. Multiplexing and gradient gene expression regulation by PROTAC-CID. (FIG. 2A) Orthogonality analysis of the PROTAC-CID systems. HEK293T cells were transfected with plasmids encoding each PROTAC-CID and UAS-Fluc, followed by treatment with indicated PROTAC or rapamycin (both cognate and noncognate pairs) for 2 days. Cells were lysed and assayed for the bioluminescence intensity. RLU, relative light units. N=3 biologically independent repeats with mean shown in the heatmap. (FIG. 2B) Diagram of dual orthogonal inducible expression system simultaneously in one cell. (left). Representative imaging of EYFP and BFP expression after dTAG^{V}-1 or dBRD9 treatment (1 μM). (FIG. 2C) Representative images of EYFP intensity in HEK293 cells of logic OR gate circuit. (FIGS. 2B and 2C) Scale bar, 125 μm. (FIG. 2D). Representative images of the logic AND gate system based on inducible DNA recombinases. Scale bar, 100 μm. (FIG. 2C-2D) HEK293 cells induced by 1 μM dBRD9 and 100 nM dTAG-13 for 2 days. (FIG. 2E) Quantitative measurement and microscopy observation of EYFP intensity of the multiple-channel gene regulation system. dTRIM24 5 M, MZ1 100 nM, Rapamycin 1 μM, dTAG-13 1 μM, dTAG^{V}-1 1 μM. Data are mean with SD. Scale bar, 125 μm. N=3 biologically independent repeats. DMSO shown as “−”.

FIGS. 3A-3H. High-induction and low-basal level gene regulation for transient genome editing. (FIG. 3A) GFP expression in HEK293T cells was measured by flow cytometry or microscopy in the presence of 100 nM dTAG-13 or DMSO. (FIG. 3B) Schematic of “three-layer” genetic circuits for tightly controlled digital output (left). Quantitative GFP intensity for measuring the Cre expression in HEK293T cells (right). (FIG. 3A) and (FIG. 3B) HEK293T cells transfected with LoxP-STOP-LoxP-GFP reporter plasmid as the control group (Ctrl). (FIGS. 3C-3E) Quantification of base editing efficiency by PROTAC-CID based inducible base editing tools in HEK293T cells driven by 100 nM dTAG-13 for 3 days. (FIG. 3C) C to T editing by inducible A3G5.13-SpCas9 (FIG. 3D) and (FIG. 3E) A to G editing by TRE3G driven or “three-layer” circuit driven ABE8e-SpG. (FIG. 3F) Schematic of the PROTAC-CID based inducible prime editing reporter platform (above). Representative images of the GFP intensity induced by dTAG-13 or DMSO after 48 h induction (below). HEK293T cells were induced in the presence of 100 nM dTAG-13 or DMSO. HEK293T cells transfected with the LoxP-STOP-LoxP-GFP reporter plasmid and the pCAG driven mutated Cre as the Control group (Ctrl). (FIG. 3G) Schematic of PROTAC-CID based inducible prime editing system. (FIG. 3H) Quantification of His₆ Tag insertion efficiency in HEK293T cells after 3 days of induction by 100 nM dTAG-13 or DMSO. The pegRNA and nicking sgRNA sequences are listed in Table 3. n=3 biologically independent repeats for all experiments. DMSO shown as “−”. Scale bar, 125 μm.

FIGS. 4A-4H. In vivo gene activation by PROTAC-CID. (FIG. 4A) Schematic of AAV-loaded PROTAC-CID system to induce Fluc gene expression. (FIG. 4B) Infecting HEK293T cells by Virus a and Virus b treated by 100 nM MZ1 or DMSO. Blank HEK293T cells as the control (Ctrl). Cells were lysed three days post-infection. (FIG. 4C) Schematic of AAV delivery and MZ1 administration routes. (FIG. 4D) Representative bioluminescence images of mice infected with AAV virus and treated with 10 mg/kg MZ1 or vehicle solution at 6 h post-MZ1 injection. (FIG. 4E) Quantification of the bioluminescence signals in (FIG. 4D). RLU, relative light units. n=3 mice. The data are mean with standard error of the mean (SEM). (FIG. 4F) Bioluminescence signals after MZ1 injection intraperitoneally (i.p. 50 mg/kg, n=4 mice) or intravenously (i.v. 10 mg/kg, n=3 mice). Data are mean with SD. (FIG. 4G-4H) Floating bar plot of bioluminescence signals (min to max) with a line at mean and polyline linking the max point at different time points, and representative images of mice after repeated i.p. injection of 50 mg/kg MZ1 (n=3 mice). Asterisks indicate statistical significance between mice receiving Virus a, Virus b, and MZ1 and mice receiving Virus b using a two-tailed t-test (*P<0.05).

FIGS. 5A-5J. Exploration of inducible split ABE system. (FIG. 5A) Schematical design of the EYFP reporter system by introducing a pre-mature stop codon in the eyfp gene. (FIG. 5B). The representative images of ABE8e-nSpCas9 transfected HEK293T cells with gRNA directed to mutate the eyfp gene or without the gRNA (Ctrl). (FIG. 5C) Crystal structure of the ABE8e deaminase domain (PDB: 6VPC). (FIGS. 5D and E) Schematical design of split ABE system by fusing FRB or FKBP3 with split ABE8e domain where FRB fused with N-terminal of ABE8e and FKBP3 fused with ABE8e-C part. (FIG. 5F and G). EYFP intensity of HEK293T cells transfected with one copy of FKBP3 or FRB fused isABE system across different splitting sites. (FIGS. 5H and 5I). Fusing two copies of FRB or FKBP3 in the isABE system. (FIG. 5J) Dose-resonse curve of isABE system under different doses of Rapamycin. N=3 biological repeats with SD.

FIGS. 6A-6D. Endogenous editing of isABE system. (FIG. 6A) Mean of the endogenous editing efficiency of isABE system compared with ABE8e-nSpCas9. (FIG. 6B). The editing efficiency with only A to be edited across five different sites. (FIGS. 6C and 6D) R-loop assay to measure the Cas9-independent off-target effect on single strand DNA (ssDNA). N=3 biological repeats with SD.

FIGS. 7A-7B. Inducible gene knockout system by isABE. (FIG. 7A) Schematical design of the isABE system to mutate the base for RNA splicing to inactivate the gene. (FIG. 7B) The expression of B2M and CD46 stained by antibodies measured by flow cytometry. N=3 biological repeats with SD.

FIGS. 8A-8B. Reporter system compatible with PAM expanded Cas9. (FIGS. 8A and 8B) Representative images of restored EYFP expression. N=3 biological repeats. The NGG PAM in FIG. 8A is SEQ ID NO: 277. The NG PAM in FIG. 8B is SEQ ID NO: 278.

FIGS. 9A-9B. NLS signal in the C terminal of FRB with increased activity. (FIG. 9A) Schematic design of the isABE system with NLS in the C terminal of FRB. (FIG. 9B) The EYFP intensity of isABE. N=3 biological repeats with SD.

FIGS. 10A-10B. PAM-expanded isABE system. (FIGS. 10A and 10B) The EYFP intensity of both NGG and NG PAM based repoter system for both of SpG fused isABE system and SpCas9 fused isABE system. The NGG PAM in FIG. 10A is SEQ ID NO: 277. The NG PAM in FIG. 10B is SEQ ID NO: 278.

FIGS. 11A-11F. Comparison of promoter configurations and PROTACs with Lenalidomide for inducible EYFP expression. (FIG. 11A) Schematic of the pUAS promoters used for evaluation of PROTAC-CID-based gene activation systems. (FIG. 11B) Quantitative EYFP gene activation efficiency for the comparison of pUAS-1 and pUAS-2 promoter-based reporter systems. (FIG. 11C) Chemical structure of dTAG-13 (Top) and Lenalidomide (Below). (FIG. 11D) and (FIG. 11E) Fluorescence quantification of EYFP intensity for dTAG-13 and molecular glue inducible gene activation in HEK293T cells. IKZF1 or IKZF3 fused to GAL4 at N-terminal or C-terminal. HEK293T cells were induced by Lenalidomide (1 μM) and 100 nM dTAG-13 for 2 days. Same data shown in FIGS. 11E and 1B for dTAG-13 group of gene activation. (FIGS. 11B, 11D, and 11E) Error bars reflect mean with SD from three biological replicates. “+” represents treatment with inducers. “−” represents treatment with DMSO. Ctrl, control. HEK293T cells transfected with reporter plasmid as the control group. (FIG. 11F) The basal level of the CID-based gene activation systems. The differences between the red column (with PROTAC-CID proteins and DMSO as the mock inducer, >=1) and the white column (untreated control samples without PROTAC-CID components, normalized as 1) represent the basal leaky effect. The p-value was calculated by a two-tailed unpaired t-test. *p<0.05; **p<0.01; ***p<0.001; n.s., non-significant. Data are from FIG. 1B, n=3 biologically independent repeats with an error bar of SD.

FIGS. 12A-12B. Truncated CRBN with disabled E3 ubiquitin ligase function. (FIG. 12A) Crystal structure of CRBN with DDB1 (Fischer et al., 2014). 7-α-helical bundle domain (HBD) in CRBN interacts with DDB1 and was labeled in the rectangle. (FIG. 12B) Performance (EYFP activation efficiency) of truncated CRBN variants compared with full-length CRBN fused with VPR for inducible gene activation treated with 100 nM dTAG-13 or DMSO shown as “−”. HEK293T cells transfected with reporter plasmids as the control group (Ctrl). Same data of full-length of CRBN group with FIG. 1B for comparison. N=3 biologically independent repeats with SD.

FIGS. 13A-13C. Sensitivity and modularity of the PROTAC-CID systems. (FIG. 13A) The dose-response curve of PROTAC small molecules as well as Rapamycin and ABA, enabling dosage-dependently tunable gene activation. The EC₅₀ of PROTAC-CID tools was calculated by Prism 9 (Graphpad) using the “[Agonist] vs. response—Variable slope (four parameters)” model. The nonlinear regression results were listed in Table 4. (FIG. 13B) dTAG-13 interacting protein partner CRBN or FKBP12^{F36V} fused with GAL4 or VPR in N-terminal or C-terminal generating eight different fusion protein with eight different pairs. EYFP induction efficiency by all different fusion pairs with either dTAG-13 (100 nM) or DMSO treatment shown as “−”. For the protein fusion configuration, unless stated otherwise, fuse protein starts with N-terminal and ends with C-terminal. The same data of group 4 with FIG. 1B for comparison. (FIG. 13C) BRD9BD fused with GAL4 in N-terminal or C-terminal treated with 1 μM dBRD9 or DMSO. Data are shown in three independent biological replications with SD.

FIGS. 14A-14G. Orthogonal analysis of the PROTAC-CID systems. (FIGS. 14A-14G) Firefly luciferase expression induced by PROTAC-CID system stimulated by 100 nM dTAG-13, 100 nM Rapamycin, 1 μM dTAG^{V}-1, 1 μM dBRD9, 100 nM MZ1, 1 μM AT1, 5 μM dTRIM24, 1 μM TL13-12, 1 M TL13-112 or Dimethyl Sulfoxide (DMSO). HEK293T cells pre-transfected with plasmids encoding GAL4 or VPR fused with protein partners. Cells were lysed 2 days post-induction and D-luciferin was added to measure the intensity of bioluminescence. RLU, relative light units.

FIGS. 15A-15B. TetR DNA binding domain for PROTAC-CID platform. (FIG. 15A) and (FIG. 15B) Schematic of the inducible PROTAC-CID system with the replacement of the DNA binding domain from GAL4 to TetR. FKBP12^{F36V} or BRD9BD fused with TetR C-terminally (Above). Efficacy of dTAG-13 or dBRD9 to induce TRE promoter driven EYFP expression. HEK293T cells transfected with plasmids treated with dTAG-13 (100 nM) or dBRD9 (1 μM) or DMSO shown as “−” for two days. N=3 biological repeats. Error bar represents the mean with SD.

FIGS. 16A-16D. Multiplexing gene regulation by PROTAC-CID small molecules. (FIG. 16A) Dual inducible expression cassettes to drive EYFP and BFP regulated by two PROTACs. (FIG. 16B) Dual inducible expression cassettes to drive the same EYFP gene forming a logic OR gate. (FIG. 16A) and (FIG. 16B) Representative images of EYFP or BFP intensity 2 days post-induction in HEK293T cells transfected with constructs in the presence of dTAG-13 (100 nM), MZ1 (100 nM), dTAG^{V}-1 (1 μM) or dBRD9 (1 μM). N=3 biologically independent repeats. (FIG. 16C) Schematic of logic AND gate by two orthogonal site-specific DNA recombinases. (FIG. 16D) Schematic design of the graded activation systems. (FIG. 16A-16B). Scale bar, 125 μm.

FIGS. 17A-17B. Inducible Cre DNA recombinase by PROTAC-CID. (FIG. 17A) Schematic of the PROTAC-CID based inducible site-specific DNA recombination platform. (FIG. 17B) Representative images and fluorescence quantification of the GFP intensity induced by dBRD9 after 2 days induction. The red bar represents the GFP intensity of induced cells, and the grey bar represents the uninduced cells transfected with PROTAC-CID system, TRE3G driven Cre and the LoxP-STOP-LoxP-GFP reporter plasmids. Control (Ctrl) group refers to the HEK293T cells transfected with the LoxP-STOP-LoxP-GFP reporter plasmid. N=3 biologically independent repeats. (FIG. 17B) Scale bar, 125 μm.

FIG. 18. Inducible PE to repair micro-deleted Cre gene. Quantitative GFP intensity for the evaluation of inducible PE activity compared with pCMV-driven PE2 in HEK293T cells. HEK293T cells transfected with LoxP-STOP-LoxP-GFP reporter plasmid as control (Ctrl). 100 nM dTAG-13 was added to the induced group. (n=3 biological repeats with SD).

FIG. 19. Schematic depicting the compact PROTAC-CID system loaded by AAV vectors to induce the Fluc expression. pEFS, elongation factor 1α short promoter. pCMV, truncated human cytomegalovirus promoter. ITR, inverted terminal repeat. In Virus a, GAL4 fused with VHL and BRD4^{BD2} fused with VP64-p65 gene activation domain was expressed constitutively. GAL4-VHL fusion protein (SEQ ID NO: 3) binds with the pUAS-2 promoter upstream of the Firefly luciferase (Fluc) gene in Virus b. In the presence of MZ1, BRD4^{BD2}-VP64-p65 will be brought in proximity to the pUAS-2 promoter to drive the Fluc gene expression.

FIG. 20. Schematic of in vivo studies of PROTAC-CID gene activation in FVB mice model treated with AAV virus and MZ1. 8-week-old FVB female mice were injected with either Virus a or Virus a and Virus b at a dose of 2×10¹⁰ genome copies (GC) per mouse by i.v. injection. 25 days post-injection, mice were administrated with MZ1 (10 mg/kg) by i.p. injection. 6 h post-MZ1 treatment, the bioluminescence was monitored. Mice were treated with 50 mg/kg MZ1 by i.p. injection or 10 mg/kg by i.v. injection to compare the route of administration. At day 52, mice were treated with 50 mg/kg MZ1 by i.p. injection and observed the luciferase bioluminescence. At day 54, after treated with 50 mg/kg MZ1 i.p. injection, liver tissue was collected for protein detection.

FIG. 21. Immunoblot analysis of endogenous BRD4 expression in FVB mice liver tissue. Immunoblot analysis of endogenous long isoform BRD4 (BRD4L) after 50 mg/kg MZ1 treatment i.p. Uncropped immunoblots are displayed in FIG. 25.

FIGS. 22A-22B. Immunoblot analysis of endogenous BRD4 expression in HEK293T cells. HEK293T cells pre-transfected with MZ1 (GAL4-VHL and BRD4^{BD2}-VPR) PROTAC-CID systems to activate the pUAS-1 driven EYFP. 48 h post-induction, the EYFP intensity was observed (FIG. 22A) and cells were lysed for immunoblot to detect the BRD4 expression. Scale bar 125 μm. (FIG. 22B). Long isoform BRD4 (BRD4L). Short isoform BRD4 (BRD4S). activation Uncropped immunoblots are displayed in FIG. 26. Rep, Replication.

FIG. 23. Weight loss analysis of AAV and MZ1 treated mice. FVB mice receiving Virus a and Virus b treated by MZ1 as in FIG. 20. The dotted line represents the mean wight of tested mice. Statistical analysis was performed using Student's t-test. NS, not significant.

FIG. 24. FACS gating examples for flow cytometry data analysis in this study. FL1-FITC-A channel used for measuring the intensity of EYFP and GFP. FL6-Pacific blue-A channel used for measuring the intensity of BFP.

FIG. 25. Uncropped original immunoblots data in FIG. 21. Mice testis and pancreas tissue as the positive control for BRD4 expression.

FIG. 26. Uncropped original immunoblots data in FIG. 22.

### DETAILED DESCRIPTION

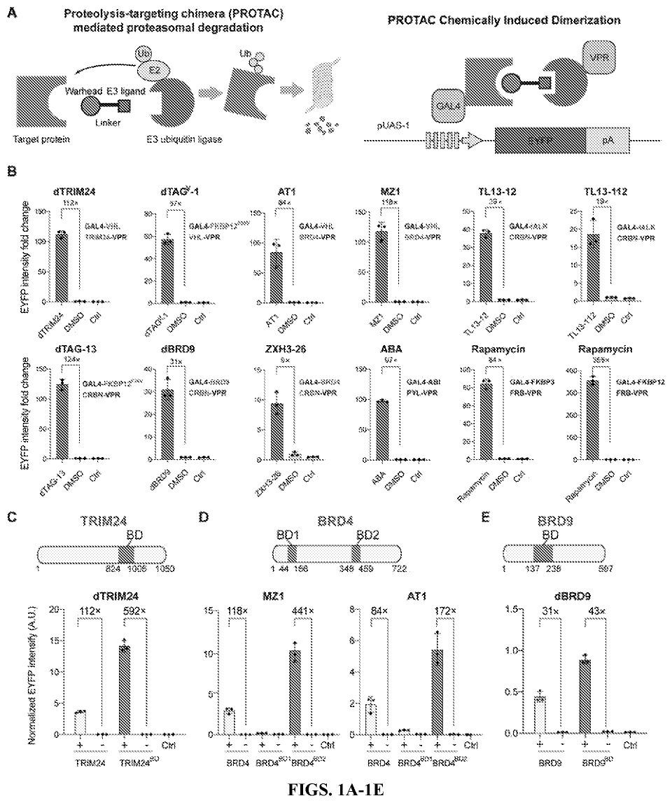

Chemically induced dimerization (CID) systems provide methods for inducible gene regulation but suffer from the limited multiplexing capability, low efficiency, and uncertainty for in vivo applications. However, CID systems have significant potential in clinical application. Proteolysis targeting chimeras (PROTACs), a rapidly growing group of small molecules that induce target protein degradation, are anticipated to become the next-generation of protein inhibitors (38). PROTACs are composed of three elements: one part (warhead) that binds to the target protein, another part that binds to an E3 ubiquitin ligase, and a linker that ties these two ligands together (38). PROTACs hijack the ubiquitin-proteasome system, causing the proximity-induced ubiquitination and degradation of the targeted protein (FIG. 1A) (39). By harnessing previously established small-molecule warheads and expanding the number of targetable proteins, at least 1600 PROTACs have been developed to date, based on a modular design strategy (40) (FIG. 1B); these contribute to an excellent and rapidly expanding repertoire. Here, the inventors present proteolysis targeting chimeras-based scalable CID (PROTAC-CID) platforms by systematically repurposing PROTAC systems for inducible gene expression regulation. Different PROTAC-CIDs are orthogonal, which allows them to be combined to fine-tune gene expression at gradient levels or multiplexing signals with different logic gating operations. When coupled with genetic circuits, the PROTAC-CID can be used for digitally inducible expression of DNA recombinases, base- and prime-editors for transient genome manipulation. The compact PROTAC-CID system can be delivered by adeno-associated viruses and elicit chemically inducible and reversible gene activation in vivo. These findings provide a versatile toolset for complex gene regulation suitable for dissecting mammalian signal transduction regulatory networks as well as gene therapy applications in therapeutic intervention.

The inventors established the PROTAC-based scalable CID platforms by systematically repurposing PROTACs for inducible transcriptional activation, enabling orthogonal, multiplexing, and digital gene regulation and safe gene therapy. Given the rapid development of PROTACs (28), the CID toolbox can be readily expanded. PROTAC protein partners are derived from human sources and could mitigate immune responses compared to ABA and other CID inducers. At least 13 PROTACs are being tested in clinical trials, while two PROTACs, ARV-110 and ARV-471, have passed phase I clinical trials with validated safety profiles and characterized pharmacological properties (29). The established safety profiles of PROTACs make them potentially suitable for inducible gene or cell therapy.

As a research tool, the effect of the repurposed PROTAC on gene expression regulation can be concurrent with the degradation of its endogenous substrate. Therefore, it is crucial to include the negative control with the same PROTAC treatment to correctly attribute the observed biological effects to gene expression regulation rather than degradation of the endogenous substrate of the PROTAC. There are many ways to minimize the interference of PROTAC-CID with the endogenous cellular process. For example, dTAG-13 and dTAG^{V}-1 work with the FKBP12^{F36V} protein partner and do not degrade wild-type FKBP12 (32, 36). Furthermore, the engineered overexpressed compact PROTAC interacting domain with higher affinity may compete with endogenous target proteins to decrease the risks of target protein depletion, as shown that the endogenous BRD4 expression was not influenced using the PROTAC-CID in both cultured cells and mice.

The highly efficient gene activation readout of the PROTAC-CID platform could make it useful for rapidly evaluating the affinity of newly constructed PROTAC candidates. While the inventors were mainly focusing on the applications of PROTAC-CID for transcriptional regulation, PROTAC-CID tools could also be applied to control protein levels directly, e.g., by dimerizing the split CRISPR/Cas effector proteins for inducible endogenous gene activation, base editing, or primer editing (30, 46, 57, 58). Thus, PROTAC-CID platforms empower PROTACs with new functionalities and exciting potential for a wide range of biomedical applications.

These and other aspects of the disclosure are set out in detail below.

### I. Proteolysis-Targeting Chimeras (PROTACS)

Proteolysis-targeting chimeras (PROTACs) are bifunctional molecules comprised of two small molecule ligands, one with high affinity towards the target protein of interest, and the second for recruitment of an E3 ligase that ubiquitinates the protein and targets it for proteolysis by the 26S proteasome (Lai and Crews, Nat. Rev. Drug Discov., 16:101-114, 2017). The two ligands are joined by a flexible tether providing a highly modular approach to generate molecules designed to degrade and silence proteins through a mechanism differing from standard small molecule or antibody inhibition. This modular approach provides room to optimize ligand affinity without concern for functional activity since silencing the protein relies on recruitment of an E3 ligase in close proximity to the protein for ubiquitination, not functional inhibition. Optimal length and hydrophobicity of the tether is important and must be empirically evaluated because if the tether is too short there may be significant steric interactions in the recruitment of the E3 ligase. Hydrophobicity of the tether should also be optimized.

Additionally, one must also consider recruitment of various E3 ubiquitin ligases and the tether length and hydrophobicity. There are three classes of E3 ligases that have been identified, which include the HECT, RING, and U-Box domain types. The HECT domain family members directly catalyze the final attachment of ubiquitin to their substrate protein, while RING and U-Box E3s do not have a direct catalytic role in protein ubiquitination (Robinson and Ardley, J. Cell Sci., 117:5191-5194, 2004; Metzger et al., J. Cell Sci., 125:531-537, 2012). The Cullin-RING ligases are the most abundant. Small molecules targeting these enzymes provide a framework to optimize ligase-recruiting molecules (Bulatov and Ciulli, Biochem J., 467:365-368, 2015). PROTACs show relatively specific target degradation and less off-target degradation than initially suggested by the ligand specificity because the E3 ligase recruited can affect the specificity of the PROTAC (Lai and Crews, Nat. Rev. Drug Discov., 16:101-114, 2017).

Exemplary PROTACs are described in the table below:

| PROTAC | Target | Target ligand | E3 ligase | E3 ligand |
|---|---|---|---|---|
| AT1 | BRD4 | JQ1 | VHL | A VH032 derivative |
| MZ1 | BRD4 | JQ1 | VHL | VHL-1 |
| dBRD9 | BRD9 | BI-7273 | CRBN | Pomalidomide |
| dBET1 | BRD2/3/4 | JQ1 | CRBN | Thalidomide |
| dTRIM24 | TRIM24 | IACS-7e | VHL | VL-269 |
| dTAG-13 | FKBP12^{F36V} | | CRBN | Thalidomide |
| TL13-12 | ALK | TAE684 | CRBN | Pomalidomide |
| TL13-112 | ALK | LDK378 | CRBN | Pomalidomide |
| ZXH3-26 | BRD4 | | CRBN | |
| dTAG^{v}-1 | FKBP12^{F36V} | | VHL | |
| CM11 | pVHL30 | | pVHL30 | |
| SNIPER(ER)-87 | ERα | 4-OHT | IAP | An LCL161 derivative |
| SNIPER(ABL)-38 | BCR-ABL | Dasatinib | IAP | An LCL161 derivative |
| SNIPER(BRD4)-1 | BRD4 | JQ1 | IAP | An LCL161 derivative |
| SNIPER(PDE4)-9 | PDE4 | A PDE4 inhibitor | IAP | An LCL161 derivative |
| HaloPROTAC3 | GFP- | Chloroalkane | VHL | A hydroxyproline |
| | HaloTag7 | | | derivative |
| PROTAC_ERRα | ERRα | A thiazolidinedione- | VHL | A hydroxyproline |
| | | based ligand | | derivative |
| PROTAC_RIPK2 | RIPK2 | A RIPK2 inhibitor | VHL | A hydroxyproline |
| | | | | derivative |
| DAS-6-2-2-6- | c-ABL | Dasatinib | VHL | A hydroxyproline |
| VHL | | | | derivative |
| DAS-6-2-2-6- | C-ABL & | Dasatinib | CRBN | Pomalidomide |
| CRBN | BCR-ABL | | | |
| BOS-6-2-2-6- | c-ABL & | Bosutinib | CRBN | Pomalidomide |
| CRBN | BCR-ABL | | | |
| ARV-771 | BRD2/3/4 | A JQ1 derivative | VHL | A HIF-1α-derived |
| | | | | (R)-hydroxyproline |
| ARV-825 | BRD2/3/4 | OTX015 | CRBN | Pomalidomide |
| dFKBP-1; | FKBP12 | Steel factor | CRBN | Thalidomide |
| dFKBP-2 | | | | |
| 3i | TBK1 | A TBK1 inhibitor | VHL | VHL ligand 2 |
| PROTAC 1 | Wild-type | Lapatinib | VHL | A hydroxyproline- |
| | EGFR | | | based ligand |
| | Exon 20 in. | | | |
| | EGFR | | | |
| | HER2 | | | |
| PROTAC 3 | Exon 19 del | Gefitinib | VHL | A hydroxyproline- |
| | EGFR | | | based ligand |
| | L858R EGFR | | | |
| PROTAC 4 | EGFR | Afatinib | VHL | A hydroxyproline- |
| | | | | based ligand |
| PROTAC 7 | c-Met | Foretinib | VHL | A hydroxyproline- |
| | | | | based ligand |
| PROTAC 12 | Sirt2 | Sirt2 inhibitor 3b | CRBN | Thalidomide |
| Compound 23 | BRD2/3/4 | HJB97 | CRBN | Lenalidomide |
| THAL-SNS-032 | CDK9 | SNS-032 | CRBN | A thalidomide |
| | | | | derivative |
| PROTAC 3 | CDK9 | An aminopyrazole | CRBN | Thalidomide |
| | | analog | | |
| TL13-117; | FLT3 | AC220 | CRBN | Pomalidomide |
| TL13-149 | | | | |
| DD-04-015 | BTK | RN486 | CRBN | Pomalidomide |
| MS4077 (5) | ALK | Ceritinib | CRBN | Pomalidomide |
| MS4078 (6) | ALK | Ceritinib | CRBN | Pomalidomide |
| Compound 42a | AR | An AR antagonist | IAP | An LCL161 derivative |
| MT-802 | Wild-type | An ibrutinib | CRBN | Pomalidomide |
| | BTK | derivative | | |
| | C481S BTK | | | |

### II. DNA Binding Domains and Promoters

Non-limiting examples of DNA binding domains are helix-turn-helix, zinc finger, leucine zipper, winged helix, winged helix turn helix, helix-loop-helix, HMG-box, Wor3 domain, immunoglobulin fold, B3 domain, TAL effector DNA-binding domains and RNA-guided DNA-binding domains. Non-limiting examples of transcription factors, from which these DNA binding domains may be derived, include Gal4, CREB, HSF, TetR, ZFHD1, Ecdysone Receptor, Nuclear Receptors, such as glucocorticoid receptor, RXR, RAR, Stat proteins, myc, Tal effectors, LexA, and the like. In one embodiment, the DNA binding domains originate from transcription factors including GAL4, ZFHD1, VP16, VP64 and NFkB (p65).

In some embodiments, the DNA binding domains may be engineered zinc finger proteins. Zinc finger proteins can be engineered to recognize any suitable target site in a promoter, such as the promoter. Methods are known in the art to design or select a zinc finger protein with high specificity and affinity to its target site and are for example described in U.S. Pat. Nos. 6,933,113, 6,933,113, 6,607,882 and 6,777,185, the contents of each of which is herein incorporated by reference in its entirety.

### III. Transcription Activators

A non-limiting example of a transactivation domains is the nine-amino-acid transactivation domain. Non-limiting examples of transcription factors from which transactivation domains may be derived from are Gal4, Oafl, Leu3, Rtg3, Pho4, Gln3, Gcn4, p53, RTg3, CREB, Gli3, E2A, HSFI, NF-IL6, myc, NFAT, BP64, B42, NF-κB and VP16, and VP64. In one embodiment, the transactivation domains originate from transcription factors including GAL4, ZFHD1, VP16, VP64 and NFkB (p65).

### IV. DNA Recombinases

Provided herein are recombinases used to impart stable, DNA-base memory to the logic and memory systems of the invention. A “recombinase,” as used herein, is a site-specific enzyme that recognizes short DNA sequence(s), which sequence(s) are typically between about 30 base pairs (bp) and 40 bp, and that mediates the recombination between these recombinase recognition sequences, which results in the excision, integration, inversion, or exchange of DNA fragments between the recombinase recognition sequences. A “genetic element,” as used herein, refers to a sequence of DNA that has a role in gene expression. For example, a promoter, a transcriptional terminator, and a nucleic acid encoding a product (e.g., a protein product) is each considered to be a genetic element.

Exemplary recombinases include, but are not limited to, Cre, Flp, Dre, SCre, VCre, Vika, B2, B3, KD, ΦC31, Bxb1, λ, HK022, HP1, γδ, ParA, Tn3, Gin, R4, TP901-1, TG1, PhiRv1, PhiBT1, SprA, XisF, TnpX, R, A118, spoIVCA, PhiMR11, SCCmec, TndX, XerC, XerD, XisA, Hin, Cin, mrpA, beta, PhiFC1, Fre, Clp, sTre, FimE, and HbiF.

Exemplary recombinase recognition sequences (RRS) include, but are not limited to, loxP, loxN, lox511, lox5171, lox2272, M2, M3, M7, M11, lox71, lox66, FRT, rox, SloxM1, VloxP, vox, B3RT, KDRT, F3, F14, attB/P, F5, F13, Vlox2272, Slox2272, SloxP, RSRT, and B2RT.

Recombinases can be classified into two distinct families: serine recombinases (e.g., resolvases and invertases) and tyrosine recombinases (e.g., integrases), based on distinct biochemical properties. Serine recombinases and tyrosine recombinases are further divided into bidirectional recombinases and unidirectional recombinases. Examples of bidirectional serine recombinases include, without limitation, β-six, CinH, ParA and γδ; and examples of unidirectional serine recombinases include, without limitation, Bxb1, ΦC31 (phiC31), TP901, TGI, φBTI, R4, cpRVI, cpFC1, MRU, A118, U153 and gp29. Examples of bidirectional tyrosine recombinases include, without limitation, Cre, FLP, and R; and unidirectional tyrosine recombinases include, without limitation, Lambda, HK101, HK022 and pSAM2. The serine and tyrosine recombinase names stem from the conserved nucleophilic amino acid residue that the recombinase uses to attack the DNA and which becomes covalently linked to the DNA during strand exchange. Recombinases have been used for numerous standard biological applications, including the creation of gene knockouts and the solving of sorting problems.

In some embodiments, the recombinases for use in the present invention are orthogonal recombinases. When a first recombinase is orthogonal to the second recombinase, it means that the second recombinase does not recognize the RRS specific for the first recombinase, neither does the first recombinase recognize the RRS specific for the second recombinase.

A recombinase can recognize multiple pairs of RRS. In some embodiments, the recombinase comprises the sequence of Cre and the corresponding recombinase recognition sequences comprise loxP. In some embodiments, the recombinase comprises the sequence of Cre and the corresponding recombinase recognition sequences comprise lox2272. In some embodiments, the recombinase comprises the sequence of Cre and the corresponding recombinase recognition sequences comprise loxN.

In some embodiments, the recombinase comprises the sequence of Bxb1 recombinase, and the corresponding recombinase recognition sequences are Bxb1 attB and Bxb1 attP. In some embodiments, the recombinase comprises the sequence of phiC31 (ϕC31) recombinase and the corresponding recombinase recognition sequences comprise phiC31 attB and phiC31 attP. In some embodiments, the recombinase comprises the sequence of Dre and the corresponding recombinase recognition sequences comprise rox. In some embodiments, the recombinase comprises the sequence of VCre and the corresponding recombinase recognition sequences comprise VloxP. In some embodiments, the recombinase comprises the sequence of VCre and the corresponding recombinase recognition sequences comprise VloxP. In some embodiments, the recombinase comprises the sequence of Flp and the corresponding recombinase recognition sequences comprise FRT. In some embodiments, the recombinase comprises the sequence of SCre and the corresponding recombinase recognition sequences comprise SloxM1. In some embodiments, the recombinase comprises the sequence of Vika and the corresponding recombinase recognition sequences comprise vox. In some embodiments, the recombinase comprises the sequence of B3 and the corresponding recombinase recognition sequences comprise B3RT. In some embodiments, the recombinase comprises the sequence of KD and the corresponding recombinase recognition sequences comprise KDRT.

### V. CRISPR Systems

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a base editing enzyme or a reverse transcriptase.

The CRISPR enzyme can be Cas9 (e.g., from S. pyogenes or S. pneumonia or S. aureus or S. auricularis or S. lugdunensis). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, a Cas9 polypeptide can be a deactivated (e.g., mutated, dCAs9) Cas9 polypeptide, wherein the deactivated Cas9 does not comprise HNH and/or RuvC nickase activities. The HNH and RuvC motifs have been characterized in S. thermophilus (see, e.g., Sapranauskas et al. Nucleic Acids Res. 39:9275-9282 (2011)) and one of skill would be able to identify and mutate these motifs in Cas9 polypeptides from other organisms. For example, the mutations D10A and H840A completely inactivate the nuclease activity of S. pyogenes Cas9. Notably, a Cas9 polypeptide in which the HNH motif and/or RuvC motif is/are specifically mutated so that the nickase activity is reduced, deactivated, and/or absent, can retain one or more of the other known Cas9 functions including DNA, RNA and PAM recognition and binding activities and thus remain functional with regard to these activities, while non-functional with regard to one or both nickase activities.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity, nucleic acid binding activity, base editing activity, or reverse transcription activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### VI. Base Editors

The engineered CRISPR technologies of base editing and prime editing have expanded the toolbox of gene editing strategies to potentially correct genetic mutations by enabling precise edits at individual nucleotides (Chemello et al., 2020). In base editing, Cas9 nickase (nCas9) or deactivated Cas9 (dCas9) is fused to a deaminase protein, allowing precise single-base pair conversions without DSBs within a defined editing window in relation to the protospacer adjacent motif (PAM) site of a sgRNA (Rees et al., 2018). There are two major classes of DNA base editors: cytosine base editors (CBEs), which convert a C:G base pair into a T:A base pair, and adenine base editors (ABEs), which convert an A:T base pair into a G:C base pair. In instances where the programmable DNA-binding domain is a CRISPR/Cas nuclease, targeted adenines lie within an “editing window” in the single-stranded (ss) DNA bubble (R-loop) induced by the CRISPR-Cas RNA-protein complex. The most commonly used ABEs comprise an adenosine deaminase heterodimer consisting of E. coli TadA (wild type) fused to an engineered E. coli TadA variant (e.g. ABEmax) or a single engineered E. coli TadA variant (e.g. ABE8e, ABE8eV106W, or ABE8.20-m) as well as a nickase Cas9 and nuclear localization sequences (NLS). ABEs have been used successfully for installation of A-to-G substitutions in multiple cell types and organisms and could potentially reverse a large number of mutations known to be associated with human disease. Examples of ABEs include those described in U.S. Pat. Publn. US20200308571, PCT Publn. WO2020214842, and PCT Publn. WO2021025750, which are each incorporated herein by reference in their entirety. Reference is made to International Publication No. WO 2018/027078, published Aug. 2, 2018; International Publication No. WO 2019/079347 published Apr. 25, 2019; International Publication No. WO 2019/226593, published Nov. 28, 2019; U.S. Patent Publication No. 2018/0073012, published Mar. 15, 2018, which issued as U.S. Pat. No. 10,113,163, on Oct. 30, 2018; and U.S. Patent Publication No. 2017/0121693, published May 4, 2017, which issued as U.S. Pat. No. 10,167,457 on Jan. 1, 2019. One of the potential concerns reported for base editors is off-target editing. The present off-target analysis did not detect any significant off-target edits in the tested sites. Base editors, such as ABEmax, can edit all available base pairs within a defined activity window.

### VII. Prime Editors

Prime editing is a versatile and precise genome editing method that directly writes new genetic information into a specified DNA site using a CRISPR system working in association with a polymerase (i.e., in the form of a fusion protein or otherwise provided in trans with the CRISPR system), wherein the prime editing system is programmed with a prime editing (pe) guide RNA (“pegRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). As such, prime editors allow for prime editing on a target nucleotide sequence in the presence of a pegRNA (or “extended guide RNA”). The pegRNA consists of (from 5′ to 3′) a sgRNA that anneals to a target site, a scaffold for the nCas9, a reverse transcription template (RT template) containing the desired edit, and a primer binding site (PBS) that binds to the non-target strand. The RT template can be programmed to introduce any type of edit, including all possible base transitions and transversions, and insertions and deletions of nucleotides of any length. The prime editing system is further enhanced by including an additional nicking sgRNA that increases editing efficiency by favoring DNA repair to replace the non-edited strand. The term “prime editor” refers to fusion constructs comprising a Cas9 nickase and a reverse transcriptase. The term “prime editor” may refer to the fusion protein or to the fusion protein complexed with a pegRNA, and/or further complexed with a second-strand nicking sgRNA. In some embodiments, the prime editor may also refer to the complex comprising a fusion protein (reverse transcriptase fused to a Cas9), a pegRNA, and a regular guide RNA capable of directing the second-site nicking step of the non-edited strand as described herein. In other embodiments, the reverse transcriptase component of the “prime editor” may be provided in trans. Further examples of prime editors and their use are provided in PCT Publn. WO2020191249, which is incorporated by reference herein in its entirety.

While INDEL profiles from CRISPR-induced DSBs may have some sequence-dependent predictability in insertion and deletion outcomes (Chakrabarti et al., 2019), the INDEL profiles are nonetheless heterogeneous in their outcome and are site-specific. NHEJ-based INDEL correction thus may produce both non-productive edits and productive edits in restoring the ORF. Prime editing has an advantage of specifying the exact insertion or deletion outcome for exon reframing, thereby ensuring that all of the edits are productive in restoring the correct ORF. Furthermore, in NHEJ-based INDEL correction, a non-productive edit prevents the sgRNA from re-annealing to the site and inducing a productive edit. In prime editing, a non-productive event (i.e. no editing as the edited strand is not successfully incorporated leaving the native sequence intact) leaves the sgRNA target site still amenable to re-annealing and another attempt at inducing the desired edit.

Prime editing can theoretically be used to correct all possible point mutations including base pair transitions and transversions, whereas base editors are limited only to transitions of A:T to G:C or C:G to T:A. In addition, theoretically prime editing is not limited to an editing window as base editing. Also, prime editing can be used to destroy splice sites. As prime editing necessitates the coordination of multiple pegRNA components for editing, such as the spacer sequence, the primer binding site (PBS), and the reverse transcriptase (RT) template, it is likely that editing events at off-target sites are minimal. However, a recent study demonstrated that two opposite strand nicks using the PE3 system can cause undesired editing outcomes in mouse zygote injections (Aida et al., 2020). These undesired editing outcomes were reduced by utilizing a sgRNA that is mutation-specific and can nick only after successful editing and resolution of the pegRNA nick (PE3b system). Nucleotide editing technologies have the potential to eliminate disease-causing mutations following a single treatment.

### VIII. Therapeutic Proteins

In some embodiments, the present application provides expression constructs encoding one or more therapeutic proteins. The therapeutic proteins that may be included in the constructs include a wide range of molecules such as cytokines, chemokines, interleukins, interferons, growth factors, coagulation factors, anti-coagulants, blood factors, bone morphogenic proteins, immunoglobulins, and enzymes. Some non-limiting examples of particular therapeutic proteins include Erythropoietin (EPO), Granulocyte colony-stimulating factor (G-CSF), Alpha-galactosidase A, Alpha-L-iduronidase, Thyrotropin α, N-acetylgalactosamine-4-sulfatase (rhASB), Dornase alfa, Tissue plasminogen activator (TPA) Activase, Glucocerebrosidase, Interferon (IF) β-1a, Interferon β-1b, Interferon γ, Interferon α, TNF-α, IL-1 through IL-36, Human growth hormone (rHGH), Human insulin (BHI), Human chorionic gonadotropin α, Darbepoetin α, Follicle-stimulating hormone (FSH), and Factor VIII.

In some embodiments, the therapeutic protein comprises a peptide sequence that is at least partially identical to any of therapeutic agent (or prophylactic agent) comprising a peptide sequence. For example, the polypeptide may comprise a peptide sequence that is at least partially identical to an antibody (e.g., a monoclonal antibody) for treating a lung disease such as lung cancer. As another example, the polypeptide may comprise a peptide sequence that is at least partially identical to a chimeric antigen receptor (CAR) expressed in an engineered immune cell.

In some embodiments, the therapeutic protein comprises a peptide or protein that restores the function of a defective protein in a subject being treated by the pharmaceutical composition described herein. For example, the polynucleotide comprises a peptide or protein that restores function of cystic fibrosis transmembrane conductance regulator (CFTR) protein, which may be used to rescue a subject who is afflicted with inborn error leading to the expression of the mutated CFTR protein. Other examples of the rescue may include administering to a subject in need thereof a polypeptide comprising a peptide or protein of wild type Dynein axonemal heavy chain 5, Dynein axonemal heavy chain 11, Bone morphogenetic protein receptor type 2, Fumarylacetoacetate hydrolase, Phenylalanine hydroxylase, Alpha-L-iduronidase, Collagen type IV alpha 3 chain, Collagen type IV alpha 4 chain, Collagen type IV alpha 5 chain, Polycystin 1, Polycystin 2, Fibrocystin (or polyductin), Solute carrier family 3 member 1, Solute carrier family 7 member 9, Paired box gene 9, Myosin VIIA, Cadherin related 23, Usherin, Clarin 1, Gap junction beta-2 protein, Gap junction beta-6 protein, Rhodopsin, dystrophia myotonica protein kinase, Dystrophin, Sodium voltage-gated channel alpha subunit 1, Sodium voltage-gated channel beta subunit 1, Coagulation factor VIII, Coagulation factor IX, N-glycanase 1, Tumor protein p53, Palmitoyl-protein thioesterase 1, Tripeptidyl peptidase 1, Kv11.1 (alpha subunit of potassium ion channel), Palmitoyl-protein thioesterase 1, ATM serine/threonine kinase, or Fibrillin 1.

### IX. AAV Vectors

Any type of vector may be used for administration of a system described herein. In some embodiments, the vector is a lipid nanoparticle. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector.

Where a vector is used, it may be a viral vector, such as a non-integrating viral vector. In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector.

In embodiments, particular embodiments, the vector is an AAV vector. AAV is a small virus that infects humans and some other primate species. AAV is not currently known to cause disease. The virus causes a very mild immune response, lending further support to its apparent lack of pathogenicity. In many cases, AAV vectors integrate into the host cell genome, which can be important for certain applications, but can also have unwanted consequences. Gene therapy vectors using AAV can infect both dividing and quiescent cells and persist in an extrachromosomal state without integrating into the genome of the host cell, although in the native virus some integration of virally carried genes into the host genome does occur. These features make AAV a very attractive candidate for creating viral vectors for gene therapy, and for the creation of isogenic human disease models. Recent human clinical trials using AAV for gene therapy in the retina have shown promise. AAV belongs to the genus Dependoparvovirus, which in turn belongs to the family Parvoviridae. The virus is a small (20 nm) replication-defective, nonenveloped virus.

Wild-type AAV has attracted considerable interest from gene therapy researchers due to a number of features. Chief amongst these is the virus's apparent lack of pathogenicity. It can also infect non-dividing cells and has the ability to stably integrate into the host cell genome at a specific site (designated AAVS1) in the human chromosome 19. This feature makes it somewhat more predictable than retroviruses, which present the threat of a random insertion and of mutagenesis, which is sometimes followed by development of a cancer. The AAV genome integrates most frequently into the site mentioned, while random incorporations into the genome take place with a negligible frequency. Development of AAVs as gene therapy vectors, however, has eliminated this integrative capacity by removal of the rep and cap from the DNA of the vector. The desired gene together with a promoter to drive transcription of the gene is inserted between the inverted terminal repeats (ITR) that aid in concatemer formation in the nucleus after the single-stranded vector DNA is converted by host cell DNA polymerase complexes into double-stranded DNA. AAV-based gene therapy vectors form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency. AAVs also present very low immunogenicity, seemingly restricted to generation of neutralizing antibodies, while they induce no clearly defined cytotoxic response. This feature, along with the ability to infect quiescent cells present their dominance over adenoviruses as vectors for human gene therapy.

The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry.

The Inverted Terminal Repeat (ITR) sequences comprise 145 bases each. They were named so because of their symmetry, which was shown to be required for efficient multiplication of the AAV genome. The feature of these sequences that gives them this property is their ability to form a hairpin, which contributes to so-called self-priming that allows primase-independent synthesis of the second DNA strand. The ITRs were also shown to be required for both integration of the AAV DNA into the host cell genome (19th chromosome in humans) and rescue from it, as well as for efficient encapsidation of the AAV DNA combined with generation of a fully assembled, deoxyribonuclease-resistant AAV particles.

With regard to gene therapy, ITRs seem to be the only sequences required in cis next to the therapeutic gene: structural (cap) and packaging (rep) proteins can be delivered in trans. With this assumption many methods were established for efficient production of recombinant AAV (rAAV) vectors containing a reporter or therapeutic gene. However, it was also published that the ITRs are not the only elements required in cis for the effective replication and encapsidation. A few research groups have identified a sequence designated cis-acting Rep-dependent element (CARE) inside the coding sequence of the rep gene. CARE was shown to augment the replication and encapsidation when present in cis.

On the “left side” of the genome there are two promoters called p5 and p19, from which two overlapping messenger ribonucleic acids (mRNAs) of different length can be produced. Each of these contains an intron which can be either spliced out or not. Given these possibilities, four various mRNAs, and consequently four various Rep proteins with overlapping sequence can be synthesized. Their names depict their sizes in kilodaltons (kDa): Rep78, Rep68, Rep52 and Rep40. Rep78 and 68 can specifically bind the hairpin formed by the ITR in the self-priming act and cleave at a specific region, designated terminal resolution site, within the hairpin. They were also shown to be necessary for the AAVS1-specific integration of the AAV genome. All four Rep proteins were shown to bind ATP and to possess helicase activity. It was also shown that they upregulate the transcription from the p40 promoter (mentioned below) but downregulate both p5 and p19 promoters.

The right side of a positive-sensed AAV genome encodes overlapping sequences of three capsid proteins, VP1, VP2 and VP3, which start from one promoter, designated p40. The molecular weights of these proteins are 87, 72 and 62 kiloDaltons, respectively. The AAV capsid is composed of a mixture of VP1, VP2, and VP3 totaling 60 monomers arranged in icosahedral symmetry in a ratio of 1:1:10, with an estimated size of 3.9 MegaDaltons.

The cap gene produces an additional, non-structural protein called the Assembly-Activating Protein (AAP). This protein is produced from ORF2 and is essential for the capsid-assembly process. The exact function of this protein in the assembly process and its structure have not been solved to date.

All three VPs are translated from one mRNA. After this mRNA is synthesized, it can be spliced in two different manners: either a longer or shorter intron can be excised resulting in the formation of two pools of mRNAs: a 2.3 kb- and a 2.6 kb-long mRNA pool. Usually, especially in the presence of adenovirus, the longer intron is preferred, so the 2.3-kb-long mRNA represents the so-called “major splice”. In this form the first AUG codon, from which the synthesis of VP1 protein starts, is cut out, resulting in a reduced overall level of VP1 protein synthesis. The first AUG codon that remains in the major splice is the initiation codon for VP3 protein. However, upstream of that codon in the same open reading frame lies an ACG sequence (encoding threonine) which is surrounded by an optimal Kozak context. This contributes to a low level of synthesis of VP2 protein, which is actually VP3 protein with additional N terminal residues, as is VP1.

Since the bigger intron is preferred to be spliced out, and since in the major splice the ACG codon is a much weaker translation initiation signal, the ratio at which the AAV structural proteins are synthesized in vivo is about 1:1:20, which is the same as in the mature virus particle. The unique fragment at the N terminus of VP1 protein was shown to possess the phospholipase A2 (PLA2) activity, which is probably required for the releasing of AAV particles from late endosomes. Muralidhar et al. reported that VP2 and VP3 are crucial for correct virion assembly. More recently, however, Warrington et al. showed VP2 to be unnecessary for the complete virus particle formation and an efficient infectivity, and also presented that VP2 can tolerate large insertions in its N terminus, while VP1 cannot, probably because of the PLA2 domain presence.

The AAV vector may be replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

### X. Nucleic Acid Delivery

In some embodiments, expression cassettes are employed for use directly in a genetic-based delivery approach. Provided herein are expression vectors which contain one or more nucleic acids encoding fusion proteins or target proteins or genes of interest. In some embodiments, a nucleic acid encoding the first fusion protein and a nucleic acid encoding the second fusion protein are provided on the same vector. In further embodiments, a nucleic acid encoding one or more of the fusion proteins and a nucleic acid encoding a gene of interest or target protein are provided on separate vectors.

Expression requires that appropriate signals be provided in the vectors and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” or “operably linked” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

### XI. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22 (3): 659-661, 2008, which is incorporated herein by reference):

$$H E D ( mg / kg ) = Animal dose ( mg / kg ) \times ( Amimal {K}_{m} / Human {K}_{m} )$$

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the patient has eaten or will eat.

### XII. Definitions

The term “nucleotide editing Cas9” refers to a Cas9 protein fused to a base editor or a prime editor. Non-limiting examples of Cas9 include SpCas9, SpCas9-NG, SaCas9, SaCas9-KKH, SauCas9, and SlugCas9. Non limiting examples of a base editor include ABEmax, ABE8e, ABE8eV106W, ABE8.20-m.

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11th ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43 (42): 13233-41). RNA and DNA have different sugar moieties and can differ by the presence of uracil or analogs thereof in RNA and thymine or analogs thereof in DNA.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

As used herein, “AAV” refers to an adeno-associated virus vector. As used herein, “AAV” refers to any AAV serotype and variant, including but not limited to an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), AAV9 vector, AAV9P vector (also known as AAVMYO, see, Weinmann et al., 2020, Nature Communications, 11:5432), and Myo-AAV vectors described in Tabebordbar et al., 2021, Cell, 184:1-20 (e.g., MyoAAV 1A, 2A, 3A, 4A, 4C, or 4E), wherein the number following AAV indicates the AAV serotype. The term “AAV” can also refer to any known AAV (vector) system. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. Structurally, AAVs are small (25 nm), single-DNA stranded non-enveloped viruses with an icosahedral capsid. Naturally occurring or engineered AAV serotypes and variants that differ in the composition and structure of their capsid protein have varying tropism, i.e., ability to transduce different cell types. When combined with active promoters, this tropism defines the site of gene expression.

“Guide RNA”, “guide RNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “guide RNA” refers to each type. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

Target sequences for Cas9s include both the positive and negative strands of genomic DNA (i.e., the sequence given and the sequence's reverse compliment), as a nucleic acid substrate for a Cas9 is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression.

An “enhancer” is a DNA sequence that can stimulate transcription activity and may be an innate element of the promoter or a heterologous element that enhances the level or tissue specificity of expression. It is capable of operating in either orientation (5′->3′ or 3′->5′) and may be capable of functioning even when positioned either upstream or downstream of the promoter.

Promoters and/or enhancers may be derived in their entirety from a native gene or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein, and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal.

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. For clarity, the terms “spacer sequence”, “spacer,” “protospacer,” “guide sequence,” or “targeting sequence” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the disclosure, such polypeptides encoded by nucleic acid sequences are not required to be identical to the endogenous protein that is defective, or whose activity, function, or expression is insufficient, deficient or absent in a treated mammal.

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors or nanoparticles to various organs and tissues.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the disclosure will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this disclosure, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.1%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### XIII. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

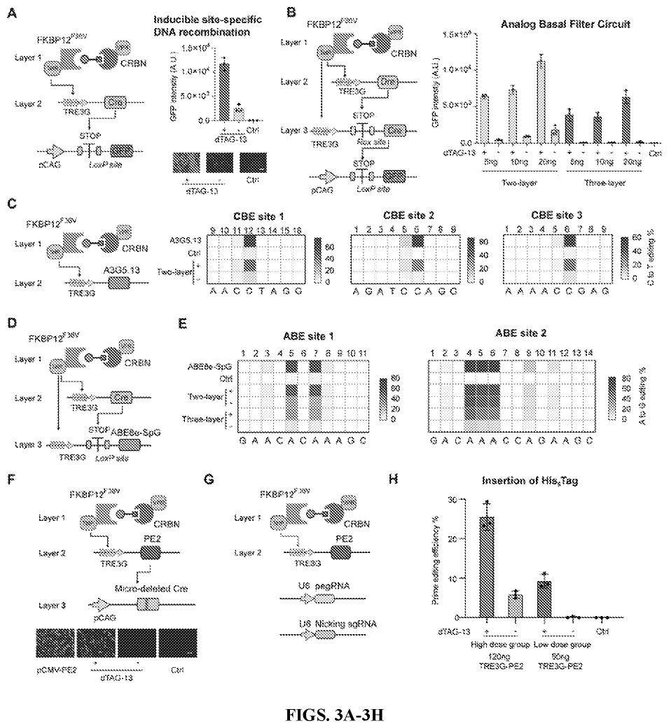

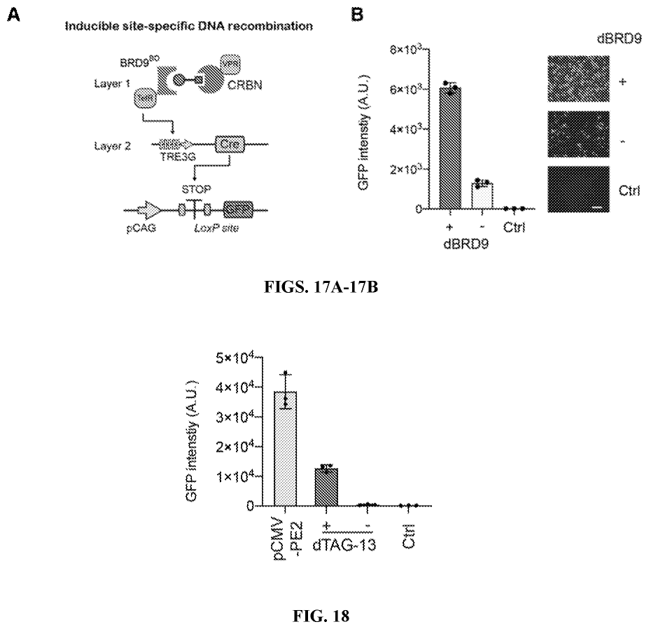

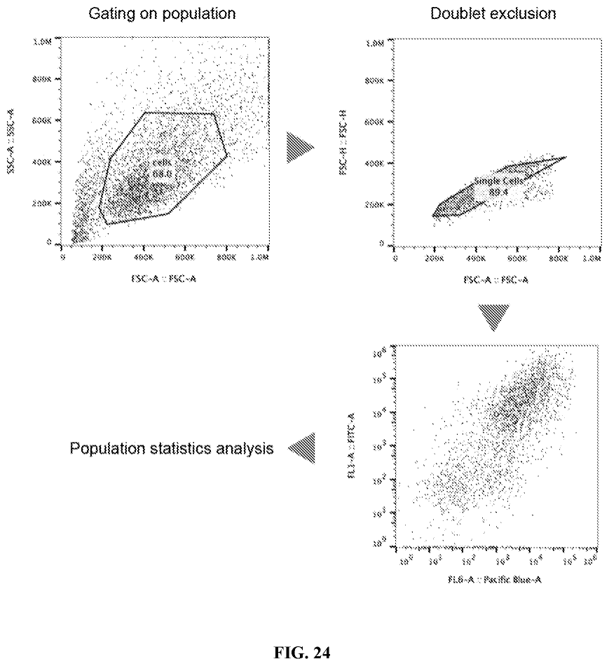

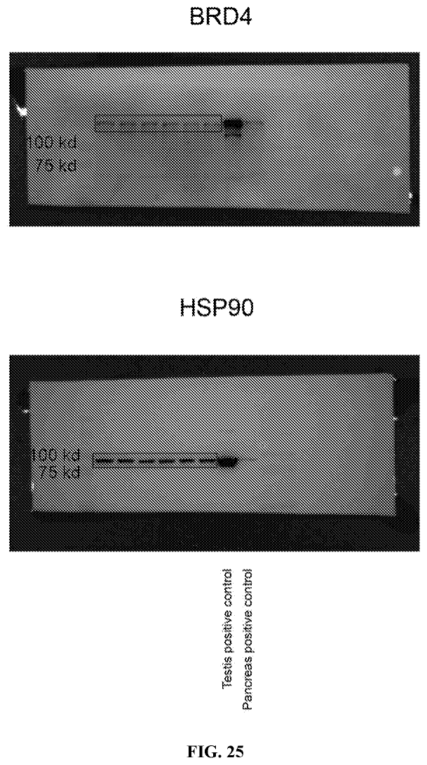

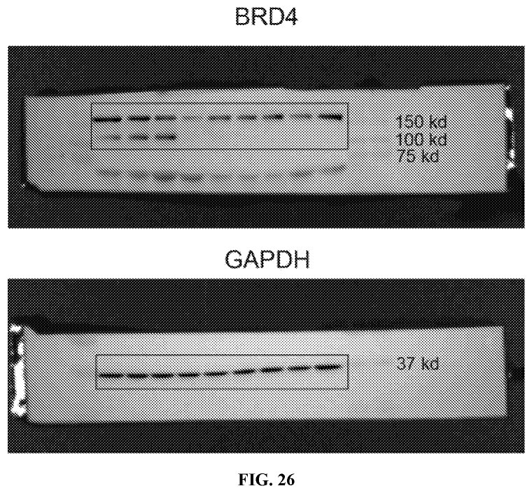

Fluorescence assay using flow cytometry analysis. Cells were treated with 0.05% Trpsin EDTA (Thermo Fisher Scientific no. 25300054) or TrypLE (Thermo Fisher Scientific no. 12605028) after transfection and centrifuged at 3000 g for 5 min. Supernatant was removed and the cell pellet was resuspended with 200 μL phosphate buffered saline (PBS) without calcium and magnesium (Thermo Fisher Scientific no. 10010023). Cells were transferred into 12×75 mm flow tubes (Global Scientific no. 110410). Cells were analyzed by MA900 flow cytometry (SONY). 405 nm laser (FL6 filter) was used for testing the BFP channel. 488 nm (FL1 filter) was used for testing the EYFP and GFP channel. Automated alignment using Automatic Setup Beads kit (SONY no. LE-B3001) is performed before running samples. In FIGS. 3A, 3B, and 17, experiments were performed under 2% FSC, 17% BSC, 24% FL1. In FIG. 18, experiments were performed under 2% FSC, 17% BSC, 27% FL1. All the other experiments were performed under 2% FSC, 17% BSC, 24% FL1 and 27% FL6. Flow cytometry data was processed and analyzed by FlowJo 10. Events were first gated with forward scatter-Area (FSC-A) and side scatter-Area (SSC-A) to select viable cells and remove the debris and dead cells. Cells were further gated with FSC-A and FSC-Height (FSC-H) for selecting single cells. The example gating strategy was shown in FIG. 24. The mean intensity of the reporter fluorescent protein (BFP and EYFP) in viable and single cell populations were calculated by FlowJo 10 as the mean of FL6-A and FL1-A. When calculating the fold change of relative fluorescence units (RFU), the relative fluorescence units of EYFP was calculated by dividing the mean of fluorescence intensity of EYFP with the mean of BFP fluorescence intensity for normalization as the following formula:

$$Normalized EYFP intensity ( A . U ) = \frac{Mean of EYFP fluorescence intensity}{Mean of BFP fluorescence intensity}$$

Next, the mean of the normalized relative fluorescence units of induced group were divided by the value of control group for calculating the fold change as the following formula:

$$EYFP intensity fold change = \frac{Mean of Normalized EYFP intensity ( A . U . ) of induced group}{\begin{matrix}Mean of Normalized EYFP intensity ( A . U . ) \\ of DMSO induced group\end{matrix}}$$

The mean of the normalized RFU of the DMSO-treated group in FIG. 11F was divided by the value of the control group for calculating the fold change using the following formula:

$$EYFP intensity fold change = \frac{\begin{matrix}Mean of normalized EYFP intensity ( A . U . ) \\ of DMSO - treated group\end{matrix}}{Mean of normalized EYFP intensity ( A . U . ) of ctrl group}$$

Plasmid construction. Nucleotide oligos were synthesized by Integrated DNA Technologies (IDT). DNA fragments were amplified by 2×Phanta Max Master Mix (Vazyme no. p515) and assembled by golden gate strategy using T4 DNA ligase (New England Biolabs no. M0202), BsaI-HFv2 (New England Biolabs no. R3733) or Esp3I (Thermo Fisher Scientific no. ER0451). Golden gate reactions were performed in 10 μL reaction volume with 1 μL T4 DNA ligase reaction buffer (New England Biolabs no. B0202S), 0.5 L BsaI-HFv2 (200 U) or 0.5 μL Esp3I (200 U) and proper volume of fragments and plasmids. Golden gate reactions were performed in a thermo cycler by the following program: 37° C. for 10 min, and 16° C. for 10 min for 10 cycles, 50° C. for 10 min and 80° C. for 10 min. CRBN fragment was amplified from plenti-UbcP-HA-CRBN-pGK-HYG plasmid, a gift from William Kaelin (Addgene plasmid #107378). FKBP12^{F36V} fragment was amplified from pLEX305_FKBP12^{F36V}-SHOC2, a gift from Andrew Aguirre (Addgene plasmid #134522). VHL fragment was amplified from pDONR223_VHL_WT, a gift from Jesse Boehm & William Hahn & David Root (Addgene plasmid #81874). BRD4 was amplified from GFP-BRD4, a gift from Kyle Miller (Addgene plasmid #65378). ALK was amplified from pDONR223-ALK, a gift from William Hahn & David Root (Addgene plasmid #23917). TRIM24 was amplified from Flag-TRIM24, a gift from Michelle Barton (Addgene plasmid #28138). Dre was amplified from pCAG-NLS-HA-Dre, a gift from Pawel Pelczar (Addgene plasmid #51272). IKZF3 was amplified from pIRIGF-IKZF3, a gift from William Kaelin (Addgene plasmid #69046). IKZF1 was amplified from pFUW-tetO-IKZF1 a gift from Filipe Pereira (Addgene plasmid #139807). SpG was amplified from pCAG-CBE4max-SpG-P2A-EGFP (RTW4552), a gift from Benjamin Kleinstiver (Addgene plasmid #139998). pCAG-loxPSTOPloxP-ZsGreen was a gift from Pawel Pelczar (Addgene plasmid #51269). PE2 was amplified from pCMV-PE2, a gift from David Liu (Addgene plasmid #132775). gRNA was constructed into the scaffold plasmid lentiGuide-Puro, a gift from Feng Zhang (Addgene plasmid #52963). For protein engineering, the 3D protein/small molecule complex or protein complex structure was visualized by UCSF Chimera v1.16. The concentration of plasmids was measured by Nanodrop Spectrophotometer (Thermo Fisher Scientific). Plasmids were sequenced by Genewiz. The protein sequences and primers are listed in the incorporated sequence listing.

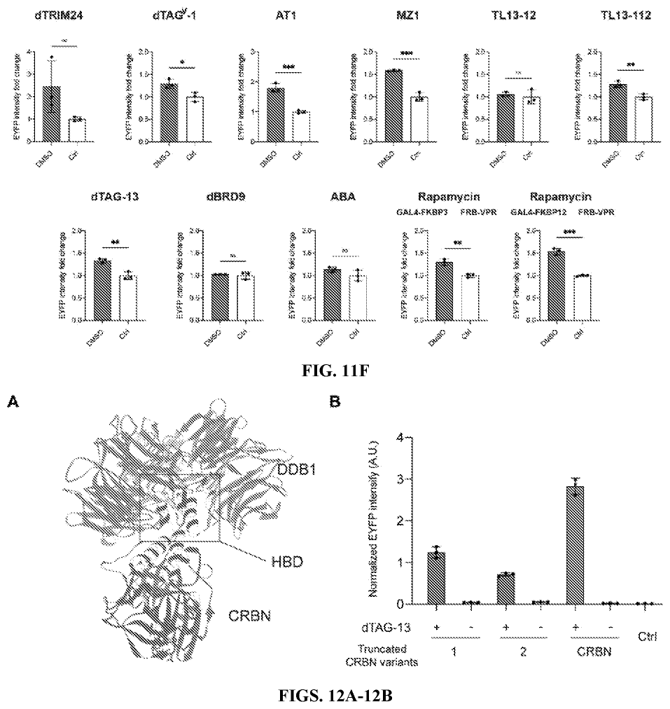

TABLE A
Primers use for plasmid cloning

| Aim | Name | SEQ ID NO: |
|---|---|---|
| GAL4-VHL | GAL4-F-d270 | 63 |
| GAL4-VHL | GAL4-R-d270 | 64 |
| GAL4-VHL | VHL-F-d270 | 65 |
| GAL4-VHL | VHL-R-d270 | 66 |
| TRIM24-VPR | TRIM24-1-F | 67 |
| TRIM24-VPR | TRIM24-1-R | 68 |
| TRIM24-VPR | TRIM24-2-F | 69 |
| TRIM24-VPR | TRIM24-m-R | 70 |
| TRIM24-VPR | TRIM24-m-F | 71 |
| TRIM24-VPR | TRIM24-2-R-c | 72 |
| TRIM24-VPR | VPR-F | 73 |
| TRIM24-VPR | VPR(L)-R | 74 |
| BRD4-VPR | BRD4-Front-F | 75 |
| BRD4-VPR | BRD4-front-R | 76 |
| BRD4-VPR | VPR-behind-F | 77 |
| BRD4-VPR | VPR-behind-R | 78 |
| GAL4-tALK | GAL4-F-d270 | 79 |
| GAL4-tALK | GAL4-R-d270 | 80 |
| GAL4-tALK | ALK-1-F-d351-c | 81 |
| GAL4-tALK | ALK-1-R-d351 | 82 |
| GAL4-tALK | ALK-2-F-d351 | 83 |
| GAL4-tALK | ALK-2-R-d351 | 84 |
| VHL-VPR | VHL-front-F | 85 |
| VHL-VPR | VHL-front-F | 86 |
| VHL-VPR | VPR-behind-F | 87 |
| VHL-VPR | VPR-behind-R | 88 |
| GAL4-BRD9 | GAL4-F | 89 |
| GAL4-BRD9 | GAL4-R | 90 |
| GAL4-BRD9 | BRD9-1-F | 91 |
| GAL4-BRD9 | BRD9-R | 92 |
| GAL4-BRD4 | BRD4-behind-F-c | 93 |
| GAL4-BRD4 | BRD4-behind-R-c | 94 |
| GAL4-BRD4 | GAL4-front-F | 95 |
| GAL4-BRD4 | GAL4-front-R | 96 |
| GAL4-ABI | ABI-F | 97 |
| GAL4-ABI | ABI-R | 98 |
| GAL4-ABI | GAL4-front-F | 99 |
| GAL4-ABI | GAL4-front-R | 100 |
| PYL-VPR | PYL-F | 101 |
| PYL-VPR | VPR(L)-R | 102 |
| GAL4-FKBP3 | FKBP3-F | 103 |
| GAL4-FKBP3 | FKBP3-R | 104 |
| GAL4-FKBP3 | GAL4-F | 105 |
| GAL4-FKBP3 | GAL4-R | 106 |
| GAL4-FKBP12 | GAL4-front-F | 107 |
| GAL4-FKBP12 | GAL4-front-R | 108 |
| GAL4-FKBP12 | FKBP12F36V- | 109 |
| | behind-F | |
| GAL4-FKBP12 | FKBP12F36V- | 110 |
| | behind-R | |
| FRB-VPR | FRB-F | 111 |
| FRB-VPR | FRB-R | 112 |
| FRB-VPR | VPR-behind-F | 113 |
| FRB-VPR | VPR-behind-R | 114 |
| TRIM24^{BD}_VPR | TRIM-F | 115 |
| TRIM24^{BD}_VPR | TRIM-R | 116 |
| TRIM24^{BD}_VPR | VPR(L)-R | 117 |
| TRIM24^{BD}_VPR | VPR-F | 118 |
| BRD4^{BD1}_VPR | BRD4-BD1-F | 119 |
| BRD4^{BD1}_VPR | VPR-R | 120 |
| BRD4^{BD2}_VPR | BRD4-BD2-F | 121 |
| BRD4^{BD2}_VPR | VPR-R | 122 |
| GAL4-BRD9^{BD} | GAL4-F | 123 |
| GAL4-BRD9^{BD} | GAL4-R | 124 |
| GAL4-BRD9^{BD} | tBRD9-F | 125 |
| GAL4-BRD9^{BD} | tBRD9-R | 126 |
| pUAS-2-Fluc | UAS-F | 127 |
| pUAS-2-Fluc | UAS-R | 128 |
| pUAS-2-Fluc | Fluc-F | 129 |
| pUAS-2-Fluc | Luci-R | 130 |
| pUAS-2-Fluc | PEST-R | 131 |
| pUAS-2-Fluc | PEST-F | 132 |
| TRE-EYFP | EYFP-F | 133 |
| TRE-EYFP | EYFP-R | 134 |
| TRE-EYFP | TRE-F | 135 |
| TRE-EYFP | TRE-R | 136 |
| pUAS-1-Dre | UAS-F | 137 |
| pUAS-1-Dre | 569-6-1-R | 138 |
| pUAS-1-Dre | dre-R | 139 |
| pUAS-1-Dre | dre-F | 140 |
| TRE3G-Cre | TRE-3G-R | 141 |
| TRE3G-Cre | TRE3G-F | 142 |
| TRE3G-Cre | TRE-cre-F | 143 |
| TRE3G-Cre | CreNLS-R-ACTA | 144 |
| TRE3G-Dre | TRE-F | 145 |
| TRE3G-Dre | Dre-R | 146 |
| TRE3G-LoxP- | CreNLS-R-ACTA | 147 |
| STOP-LoxP-Cre | | |
| TRE3G-LoxP- | TRE-F | 148 |
| STOP-LoxP-Cre | | |
| TRE3G-LoxP- | TRE3G-F | 149 |
| STOP-LoxP-Cre | | |
| TRE3G-LoxP- | TRE-3G-R | 150 |
| STOP-LoxP-Cre | | |
| TRE3G-LoxP- | A3G-1-F | 151 |
| STOP-LoxP-Cre | | |
| TRE3G-LoxP- | A3G-m-R | 152 |
| STOP-LoxP-Cre | | |
| TRE3G-LoxP- | A3G-m-F | 153 |
| STOP-LoxP-Cre | | |
| TRE3G-LoxP- | A3G-2-R | 154 |
| STOP-LoxP-Cre | | |
| TRE3G-LoxP- | A3G-3-F | 155 |
| STOP-LoxP-Cre | | |
| TRE3G-LoxP- | A3G-3-R | 156 |
| STOP-LoxP-Cre | | |
| TRE3G-ABE8e-SpG | TRE3G-F | 157 |
| TRE3G-ABE8e-SpG | TRE-3G-R- | 158 |
| | GACC | |
| TRE3G-ABE8e-SpG | ABE(TRE-F)- | 159 |
| | gacc | |
| TRE3G-ABE8e-SpG | SpG-1-R | 160 |
| TRE3G-ABE8e-SpG | SpG-2-F | 161 |
| TRE3G-ABE8e-SpG | SpG-2-R | 162 |
| TRE3G-LoxP- | TRE3G-F | 163 |
| STOP-LoxP- | | |
| ABE8e-SpG | | |
| TRE3G-LoxP- | TRE-3G-R- | 164 |
| STOP-LoxP- | GACC | |
| ABE8e-SpG | | |
| TRE3G-LoxP- | loxP-F | 165 |
| STOP-LoxP- | | |
| ABE8e-SpG | | |
| TRE3G-LoxP- | loxP-R | 166 |
| STOP-LoxP- | | |
| ABE8e-SpG | | |
| TRE3G-LoxP- | ABE-F | 167 |
| STOP-LoxP- | | |
| ABE8e-SpG | | |
| TRE3G-LoxP- | SpG-1-R | 168 |
| STOP-LoxP- | | |
| ABE8e-SpG | | |
| TRE3G-LoxP- | SpG-2-F | 169 |
| STOP-LoxP- | | |
| ABE8e-SpG | | |
| TRE3G-LoxP- | SpG-2-R | 170 |
| STOP-LoxP- | | |
| ABE8e-SpG | | |
| TRE3G-PE2 | TRE3G-R | 171 |
| TRE3G-PE2 | TRE3G-F | 172 |
| TRE3G-PE2 | Protac-PE-F | 173 |
| TRE3G-PE2 | Cas9-SpG-R | 174 |
| TRE3G-PE2 | Cas9-SpG-F | 175 |
| TRE3G-PE2 | PE-1-R | 176 |
| TRE3G-PE2 | PE-2-F | 177 |
| TRE3G-PE2 | PE-4-R | 178 |
| microdeleted Cre | Cre-F | 179 |
| microdeleted Cre | Cre-deletion-1-R | 180 |
| microdeleted Cre | Cre-deletion-2-F | 181 |
| microdeleted Cre | Cre-R | 182 |
| cre pegRNA | his6-U6-F | 183 |
| cre pegRNA | His6-U6-R | 184 |
| His pegRNA | his6-U6-F | 185 |
| His pegRNA | His6-U6-R | 186 |
| Virus a | EFS-F | 187 |
| Virus a | EFS-R | 188 |
| Virus a | VHL-R-d550 | 189 |
| Virus a | GAL4-F | 190 |
| Virus a | CMV-R57 | 191 |
| Virus a | CMV-For-d459- | 192 |
| | v2 | |
| Virus a | P65-R | 193 |
| Virus a | BD2-F | 194 |
| Virus a | P65-R | 195 |
| Virus a | BD2-F | 196 |
| pUAS-1-EYFP | mini3G-F | 197 |
| pUAS-1-EYFP | EYFP-R | 198 |
| Plasmids in | GAL4-F | 199 |
| FIG. 11C and 1D | | |
| Plasmids in | GAL4-R | 200 |
| FIG. 11C and 1D | | |
| Plasmids in | ikzf1-F | 201 |
| FIG. 11C and 1D | | |
| Plasmids in | ikzf1-R | 202 |
| FIG. 11C and 1D | | |
| Plasmids in | IZKF3-F | 203 |
| FIG. 11C and 1D | | |
| Plasmids in | izkf3-R | 204 |
| FIG. 11C and 1D | | |
| Plasmids in | GAL4-F | 205 |
| FIG. 11C and 1D | | |
| Plasmids in | GAL4-R | 206 |
| FIG. 11C and 1D | | |
| Plasmids in | ikzf1-F | 207 |
| FIG. 11C and 1D | | |
| Plasmids in | ikzf1-R | 208 |
| FIG. 11C and 1D | | |
| Plasmids in | IZKF3-F | 209 |
| FIG. 11C and 1D | | |
| Plasmids in | izkf3-R | 210 |
| FIG. 11C and 1D | | |
| Plasmids in | CRBN-behind-F | 211 |
| FIG. 12B | | |
| Plasmids in | CRBN-behind-R | 212 |
| FIG. 12B | | |
| Plasmids in | CRBN-Front-F | 213 |
| FIG. 12B | | |
| Plasmids in | CRBN-Front-R | 214 |
| FIG. 12B | | |
| Plasmids in | FKBP12F36V- | 215 |
| FIG. 12B | behind-F | |
| Plasmids in | FKBP12F36V- | 216 |
| FIG. 12B | behind-R | |
| Plasmids in | FKBP12F36V- | 217 |
| FIG. 12B | front-F | |
| Plasmids in | FKBP12F36V- | 218 |
| FIG. 12B | front-R | |
| Plasmids in | GAL4-behind-F | 219 |
| FIG. 12B | | |
| Plasmids in | GA14-behind-R | 220 |
| FIG. 12B | | |
| Plasmids in | GAL4-front-F | 221 |
| FIG. 12B | | |
| Plasmids in | GAL4-front-R | 222 |
| FIG. 12B | | |
| Plasmids in | VPR-front-F | 223 |
| FIG. 12B | | |
| Plasmids in | VPR-front-R | 224 |
| FIG. 12B | | |
| Plasmids in | VPR-behind-F | 225 |
| FIG. 12B | | |
| Plasmids in | VPR-behind-R | 226 |
| FIG. 12B | | |
| BRD9^{BD}-GAL4 | d565-GAL4-F | 227 |
| BRD9^{BD}-GAL4 | GAL4-R | 228 |
| BRD9^{BD}-GAL4 | BRD9BD-F | 229 |
| BRD9^{BD}-GAL4 | BRD9D-R | 230 |
| TetR-FKBP12^{F36V} | tetR-1-F | 231 |
| TetR-FKBP12^{F36V} | tetR-1-R | 232 |
| TetR-FKBP12^{F36V} | tetR-2-F | 233 |
| TetR-FKBP12^{F36V} | tetR-2-R | 234 |
| TetR-FKBP12^{F36V} | FKBP12F36V- | 235 |
| | behind-F | |
| TetR-FKBP12^{F36V} | FKBP12F36V- | 236 |
| | behind-R | |
| TetR-BRD9^{BD} | tetR-1-F | 237 |
| TetR-BRD9^{BD} | tetR-1-R | 238 |
| TetR-BRD9^{BD} | tetR-2-F | 239 |
| TetR-BRD9^{BD} | tetR-2-R | 240 |
| TetR-BRD9^{BD} | tBRD9-behind-F | 241 |
| TetR-BRD9^{BD} | tBRD9-behind-R | 242 |
| tCRBN-1-VPR | CRBN-v7-F-c55 | 243 |
| tCRBN-1-VPR | VPR-behind-R | 244 |
| tCRBN-2-VPR | CRBN-2-F-d420 | 245 |
| tCRBN-2-VPR | VPR-behind-R | 246 |
| tCRBN-2-VPR | CRBN-1-R-d420 | 247 |
| tCRBN-2-VPR | CRBN-Front-F | 248 |

Transfection and microscopy. HEK293T cells (American Type Culture Collection, no. CRL-3216) were cultured with high-glucose Dulbecco's modified Eagle's medium (DMEM) (Thermo Fisher Scientific no. 10569044) with 10% fetal bovine serum (FBS) (Thermo Fisher Scientific no. 10437028) and 1×penicillin-streptomycin (Thermo Fisher Scientific no. 15140122) at 37° C. with 5% CO₂. Except for the data in FIGS. 3C, 3E, and 3H, cells were plated into 96 well plate (Corning no. 3598) and transfected by Polyethyleneimine Max (PEI Max) (Polysciences no. 24765-1). 1.5 μL PEI Max (1 mg/mL, pH=7.1) and 100 μl DMEM were mixed with plasmids to be slowly dropped into the cell medium after 30 mins of incubation. Medium was changed 12 hours post-transfection. The inductive small molecules, including Rapamycin (STEM CELL technologies no. 73362), dTAG-13 (TOCRIS no. 6605/5), dTAG^{V}-1 (TOCRIS no. 6744-5), dBRD9 (TOCRIS no. 6606/5), dTRIM24 (TOCRIS no. 6607/5), MZ1 (TOCRIS no. 6154/5), AT1 (TOCRIS no. 6356/5), TL13-12 (TOCRIS no. 6744/5), TL13-112 (TOCRIS no. 6745/5), dTAG-7 (TOCRIS no. 69125), ZXH3-26 (TOCRIS no. 6713/5) and ABA (GoldBio no. A-050-100), dissolved in Dimethyl sulfoxide (DMSO) (Sigma, no. D8418) were added. For the microscopy observation, the images were taken by Microscopy EVOS (Life Technologies) and processed by ImageJ (Schneider et al., 2012). If not otherwise noted, 60 ng of each plasmid were used for each sample. The transfection plasmid configuration is listed in Table 1.

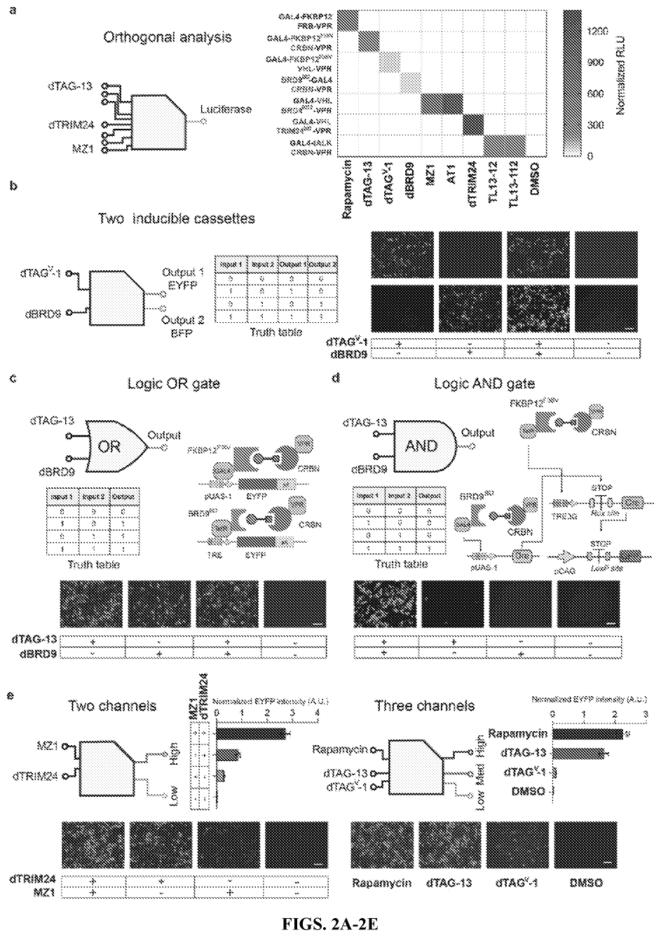

TABLE 1
Transfection plasmid configuration
Plasmid used in FIG. 2D

| | 1 | | 2 | | 3 | | 4 | |
|---|---|---|---|---|---|---|---|---|
| pCAG-LoxP- | 50 | ng | 50 | ng | 50 | ng | 50 | ng |
| STOP-LoxP- | | | | | | | | |
| GFP(ZSGREEN) | | | | | | | | |
| pUAS-1-Dre | 1 | ng | 1 | ng | 1 | ng | 1 | ng |
| TRE3G-loxP- | 1 | ng | 1 | ng | 1 | ng | 30 | ng |
| STOP-LoxP-Cre | | | | | | | | |
| pCAG-TetR- | 50 | ng | 50 | ng | 50 | ng | 50 | ng |
| FKBP12\hat{ }F36V | | | | | | | | |
| pCAG-CRBN- | 100 | ng | 100 | ng | 100 | ng | 100 | ng |
| VPR | | | | | | | | |
| pCAG-TetR- | 50 | ng | 50 | ng | 50 | ng | 50 | ng |
| BRD9\hat{ }BD | | | | | | | | |

Plasmid used in FIGS. 17 and 3A

| | 1 | 2 | 3 | 4 | 5 |
|---|---|---|---|---|---|
| pCAG-LoxP- | 60 ng | 60ng | 60 ng | 60 ng | 60 ng |
| STOP-LoxP- | | | | | |
| GFP(ZSGREEN) | | | | | |
| TRE3G-Cre | 10 ng | 10 ng | 10 ng | 10 ng | |
| pCAG-TetR- | 60 ng | 60 ng | | | |
| FKBP12\hat{ }F36V | | | | | |
| pCAG-CRBN- | 60 ng | 60 ng | 60 ng | 60 ng | |
| VPR | | | | | |
| pCAG-TetR- | | | 60 ng | 60 ng | |
| BRD9\hat{ }BD | | | | | |
| Condition: | Treated | Treated | Treated | Treated | |
| | with | with | with | with | |
| | dTAG-13 | DMSO | dBRD9 | DMSO | |

Plasmid used in FIG. 3B

| | 1 | 2 | 3 | 4 |
|---|---|---|---|---|
| pCAG-LoxP- | 50 ng | 50 ng | 50 ng | 50 ng |
| STOP-LoxP- | | | | |
| GFP(ZSGREEN) | | | | |
| TRE3G-Dre | | 5 ng | 5 ng | 10 ng |
| TRE3G-Rox- | | 5 ng | 5 ng | 10 ng |
| STOP-Rox-Cre | | | | |
| pCAG-TetR- | | 30 ng | 30 ng | 30 ng |
| FKBP12\hat{ }F36V | | | | |
| pCAG-CRBN- | | 30 ng | 30 ng | 30 ng |
| VPR | | | | |
| TRE3G-Cre | | | | |
| Condition: | | Treated with | Treated with | Treated with |
| | | dTAG-13 | DMSO | dTAG-13 |

| | 5 | 6 | 7 | 8 |
|---|---|---|---|---|
| pCAG-LoxP- | 50 ng | 50 ng | 50 ng | 50 ng |
| STOP-LoxP- | | | | |
| GFP(ZSGREEN) | | | | |
| TRE3G-Dre | 10 ng | 20 ng | 20 ng | |
| TRE3G-Rox- | 10 ng | 20 ng | 20 ng | |
| STOP-Rox-Cre | | | | |
| pCAG-TetR- | 30 ng | 30 ng | 30 ng | 30 ng |
| FKBP12\hat{ }F36V | | | | |
| pCAG-CRBN- | 30 ng | 30 ng | 30 ng | 30 ng |
| VPR | | | | |
| TRE3G-Cre | | | | 5 ng |
| Condition: | Treated | Treated | Treated | Treated |
| | with | with | with | with |
| | DMSO | dTAG-13 | DMSO | dTAG-13 |

| | 9 | 10 | 11 | 12 | 13 |
|---|---|---|---|---|---|
| pCAG-LoxP- | 50 ng | 50 ng | 50 ng | 50 ng | 50 ng |
| STOP-LoxP- | | | | | |
| GFP(ZSGREEN) | | | | | |
| TRE3G-Dre | | | | | |
| TRE3G-Rox- | | | | | |
| STOP-Rox-Cre | | | | | |
| pCAG-TetR- | 30 ng | 30 ng | 30 ng | 30 ng | 30 ng |
| FKBP12\hat{ }F36V | | | | | |
| pCAG-CRBN- | 30 ng | 30 ng | 30 ng | 30 ng | 30 ng |
| VPR | | | | | |
| TRE3G-Cre | 5 ng | 10 ng | 10 ng | 20 ng | 20 ng |
| Condition: | Treated | Treated | Treated | Treated | Treated |
| | with | with | with | with | with |
| | DMSO | dTAG-13 | DMSO | dTAG-13 | DMSO |

Plasmid used in FIG. 3C

| | 1 | 2 | 3 |
|---|---|---|---|
| TRE3G-A3G5.13 | | 100 ng | 100 ng |
| pCMV-A3G5.13 | 100 ng | | |
| gRNA | 50 ng | 50 ng | 50 ng |
| pCAG-TetR- | | 30 ng | 30 ng |
| FKBP12\hat{ }F36V | | | |
| pCAG-CRBN- | | 30 ng | 30 ng |
| VPR | | | |
| TRE3G-Cre | | | |
| Condition: | | Treated with | Treated with |
| | | dTAG-13 | DMSO |

Plasmid used in FIG. 3E

| | 1 | 2 | 3 | 4 | 5 | 6 |
|---|---|---|---|---|---|---|
| TRE3G-LoxP- | | | | 50 ng | 50 ng | |
| STOP-LoxP- | | | | | | |
| ABE8e-SpG | | | | | | |
| TRE3G-ABE8e- | | 50 ng | 50 ng | | | |
| SpG | | | | | | |
| pCMV-ABE8e- | 50 ng | | | | | |
| SpG | | | | | | |
| gRNA | 30 ng | 30 ng | 30 ng | 30 ng | 30 ng | |
| TRE3G-Cre | | | | 5 ng | 5 ng | |
| pCAG-TetR- | | 30 ng | 30 ng | 30 ng | 30 ng | |
| FKBP12\hat{ }F36V | | | | | | |
| pCAG-CRBN- | | 30 ng | 30 ng | 30 ng | 30 ng | |
| VPR | | | | | | |
| Condition: | | Treated | Treated | Treated | Treated | |
| | | with | with | with | with | |
| | | dTAG-13 | DMSO | dTAG-13 | DMSO | |

Plasmid used in FIG. 3I

| | 1 | 2 | 3 | 4 | 5 |
|---|---|---|---|---|---|
| TRE3G-PE2 | 120 ng | 120 ng | 50 ng | 50 ng | |
| pegRNA | 40 ng | 40 ng | 60 ng | 60 ng | |
| nicking sgRNA | 15 ng | 15 ng | 60 ng | 60 ng | |
| pCAG-TetR- | 40 ng | 40 ng | 60 ng | 60 ng | |
| FKBP12\hat{ }F36V | | | | | |
| pCAG-CRBN- | 40 ng | 40 ng | 60 ng | 60 ng | |
| VPR | | | | | |
| Condition: | Treated | Treated | Treated | Treated | |
| | with | with | with | with | |
| | dTAG-13 | DMSO | dTAG-13 | DMSO | |

Plasmid used in FIG. 3G

| | 1 | 2 | 3 | 4 |
|---|---|---|---|---|
| pCAG-LoxP- | 60 ng | 60 ng | 60 ng | 60 ng |
| STOP-LoxP- | | | | |
| GFP(ZSGREEN) | | | | |
| TRE3G-PE2 | | 10 ng | 10 ng | |
| pCMV-PE2 | 10 ng | | | |
| pegRNA | 60 ng | 60 ng | 60 ng | |
| pCAG- | 5 ng | 5 ng | 5 ng | 5 ng |
| microdeleted Cre | | | | |
| nicking sgRNA | 60 ng | 60 ng | 60 ng | |
| pCAG-TetR- | | 30 ng | 30 ng | |
| FKBP12\hat{ }F36V | | | | |
| pCAG-CRBN- | | 30 ng | 30 ng | |
| VPR | | | | |
| Condition: | | Treated with | Treated with | |
| | | dTAG-13 | DMSO | |

Luciferase luminescence intensity measurement. Before transfection, HEK293T cells were seeded in clear bottom 96 well assay plates (Corning no. 3610) with 100 μL DMEM with 10% FBS and 1× penicillin-streptomycin at 37° C. with 5% CO₂. When cells reached 50% confluency, 1.5 μL PEI Max (1 mg/mL, pH=7.1) and 100 μL DMEM were mixed with 60 ng of each plasmid (GAL4 and VPR fused target protein or E3 ubiquitin ligase, pUAS-2-Fluc) for 30 min at room temperature. Mixture was transferred into cells gently. 12 h after transfection, medium was changed, and the inductive small molecules were supplied. 2-days post induction, 120 μL of medium was aspirated from each well containing 200 μL of medium, leaving 80 μL of medium per well, then treated with 40 μL of lysis buffer (500 mM DTT (Sigma no. D0632), 10 mM coenzyme A (RPI no. C95275), 100 mM ATP (Thermo Fisher Scientific no. R1441), 80 mg/mL D-luciferin (GoldBio no. LUCNA-100), Triton lysis buffer (0.1082 M Tris-HCl, 0.0419 M Tris-Base, 75 mM NaCl, 3 mM MgCl₂)). Plate was shaken with 20 seconds in “Orbital” mode with frequency of 432 rpm and Amplitude of 1 mm to lysis the cells. Recording the luminescence by plate reader Infinite M200 (TECAN) with 1000 ms integration time using Megellan v1.7 (TECAN). Mean of luciferase luminescence relative light units (RLU) was calculated by the average of three biological replications. Normalized RLU were calculated by dividing RLU of the tested group with mean of RLU of DMSO treated control group for normalization:

$$Normalized RLU = \frac{RLU of testing sample}{Mean of DMSO treated control group}$$

Base editing measurement. Before transfection, HEK293T cells were plated into 96 well plates (Corning no. 3598) with 20% confluency. 0.5 μL Lipofectamine 2000 (Life Technology no. 11661089) was mixed with 25 μL DMEM for 5 mins incubation. 80 ng to 210 ng Plasmids (See Table 1 for plasmid dosage used in each condition) were then mixed with 25 μL DMEM and added into the Lipofectamine 2000 and DMEM mixture for 20 min incubation at the room temperature. Mixture was added into cells gently. After 12 h, supernatants were changed with fresh 10% FBS (Thermo Fisher Scientific no. 10437028) DMEM medium (Thermo Fisher Scientific no. 10569044), Puromycin (10 μg/mL Thermo Fisher Scientific no. A1113803) and supplied with 100 nM dTAG-13 (TOCRIS no. 6605/5) or DMSO (Sigma, no. D8418). After 3 days, cell medium was removed and cells were treated with 100 μL lysis buffer (10 mM tris-HCl (pH=7.5), 0.05% SDS, and proteinase K (25 μg/mL, Thermo Fisher Scientific no. 01169965)) followed by 37° C. 1 h, 58° C. 30 min and inactivated at 95° C. for 20 min. The cell lysis was amplified by 2×Phanta Max Master Mix (Vazyme no. p515) following the program: 95° C. 3 min, 95° C. 15s and 58° C. 15s with 72° C. for 35 cycles, and 72° C. 5 min. The guide RNA sequences and primers were listed in Table 2. The editing efficiency was measured by Sanger sequencing and analyzed by EditR (https://moriaritylab.shinyapps.io/editr_v10/) (Kluesner et al., 2018).

TABLE 2
gRNA sequences and primers for amplifying the genome sites in FIGS. 3C and 3E

| Description | gRNA sequence | Forward primer | Reverse primer |
|---|---|---|---|
| A3G site 1 | GTTACGAAAACCTA | TGAAAGTGGCATCT | ACCCTTGCATTCCA |
| | GGGGTG (SEQ ID NO: | TGAAAGGG (SEQ ID | ATACCAC (SEQ ID |
| | 254) | NO: 255) | NO: 256) |
| A3G site 2 | AGATCCAGGGACAC | GTGGGAAACAGCCG | CACTGAGCACTGAA |
| | GGTGCT (SEQ ID NO: | TCAG (SEQ ID NO: | GGCC (SEQ ID NO: |
| | 257) | 258) | 259) |
| A3G site 3 | AAAACCGAGGGGTA | ACACTCTTTCCCTAC | GACTGGAGTTCAGA |
| | AGAATC (SEQ ID NO: | ACGACGCTCTTCCG | CGTGTGCTCTTCCG |
| | 260) | ATCTATAGGATAGG | ATCTCTGCTGCTCCT |
| | | AGTGATGGACAGG | CAATACACC (SEQ ID |
| | | (SEQ ID NO: 261) | NO: 262) |
| ABE site 1 | GACAAACCAGAAGC | TCTCTTGTGGTTTCC | ACTTTCCCCTGAGTT |
| | CGCTCC (SEQ ID NO: | TAGCTTCTGA (SEQ | TAAGTGATG (SEQ ID |
| | 263) | ID NO: 264) | NO: 265) |
| ABE site 2 | GAACACAAAGCATA | ACATTTGGGCTTCTT | CCTGATGTAATGAC |
| | GACTGC (SEQ ID NO: | TCTAGTTGA (SEQ ID | TAGACTGAGGC |
| | 266) | NO: 267) | (SEQ ID NO: 268) |

Prime editing measurement. HEK293T cells were seeded into 96 well plates (Corning no. 3598). When the cells reach 20% confluency, 265 ng to 290 ng plasmids (See Table 1 for plasmid dosage used in each condition) were firstly mixed with 25 μL DMEM. 0.5 μL Lipofectamine 2000 (Life Technology no. 11661089) was incubated with 25 μL DMEM for 5 min. Next, plasmid solution was mixed with the Lipofectamine 2000 solution for 20 min and added into the cells gently. After 12 h, the supernatants were changed with fresh 10% FBS (Thermo Fisher Scientific no. 10437028) DMEM medium (Thermo Fisher Scientific no. 10569044) supplied with Puromycin (10 μg/mL Thermo Fisher Scientific no. A1113803) and 100 nM dTAG-13 (TOCRIS no. 6605/5) or DMSO (Sigma, no. D8418). 3 days post-induction, the supernatant was removed and supplied with 100 μL lysis buffer (10 mM tris-HCl (pH=7.5), 0.05% SDS, and proteinase K (25 μg/mL, Thermo Fisher Scientific no. 01169965)) followed by 37° C. 1 h, 58° C. 30 min and inactivated at 95° C. for 20 min. To design primers to amplify the editing region with clear band, the gRNA target sequences as the inquiry by the BLAT Search Genome tool (https://genome.ucsc.edu/cgi-bin/hgBlat). The 2000 base pair (bp) flanking genomic DNA sequences was downloaded, and the primers were designed by Geneious Prime 3 (Biomatter). 0.5 μL cell lysis were amplified with DNA primers (listed in Table 3) by 2×Phanta Max Master Mix (Vazyme no. p515) following the program: 95° C. 3 min, 95° C. 15s and 58° C. 15s with 72° C. for 35 cycles, and 72° C. 5 min. The fragments were cleaned by PB buffer (Qiagen no. 166021045). 10 ng PCR products were used for Sanger sequencing (Genewiz). The insertion efficiency was analyzed online in TIDE (https://tide.nki.nl/) (Brinkman et al., 2018) with the setting (left boundary=100, Decomposition window (115-685 bp), Indel size range (28 bp), P-value threshold=0.001).

TABLE 3
pegRNA sequences, nicking sgRNA and primers for measuring base editing
efficiency in FIGS. 3H

| | | | PBS | RT |
|---|---|---|---|---|
| | | | length | template |
| pegRNA | space sequence | 3′ extension | (nt) | length (nt) |
| HEK3_His₆ins | GGCCCAGACTG | TGGAGGAAGCAGGGCTT | 13 | 52 |
| | AGCACGTGA | CCTTTCCTCTGCCATCAA | | |
| | (SEQ ID NO: 269) | TGATGGTGATGATGGTG | | |
| | | CGTGCTCAGTCTG (SEQ | | |
| | | ID NO: 270) | | |
| Cre_2ATins | AAATGCCAGAT | TCGCTGCCAGGATATAC | 11 | 14 |
| | TACGTATCC | GTAATCTGGC (SEQ ID | | |
| | (SEQ ID NO: 271) | NO: 272) | | |

| Nicking sgRNA | spacer sequence |
|---|---|
| HEK3_His₆ins | GTCAACCAGTATCCCGGTGC (SEQ ID NO: 273) |
| Cre_2ATins | CGAACGCACTGATTTCGACC (SEQ ID NO: 274) |
| Description | Sequence |
| HEK3 fwd | CTTTTCCTCTGTTGAGCTCG (SEQ ID NO: 275) |
| HEK3 rev | GAATCAGTGCTGGAGAATGG (SEQ ID NO: 276) |

Immunoblots and RT-qPCR. Mice liver tissues or culture cells were lysed in RIPA buffer (Abcam no. ab156034) supplemented with phosphatase inhibitor (Thermo Fisher Scientific no. PIA32957) and protease inhibitors (Fishers cientific no. A32965). Lysates were resolved by 10% Tris-glycine SDS-PAGE, transferred to PVDF membrane (Bio-Rad no. 1620177), and blotted with antibodies BRD4 (Abcam, ab128874), GAPDH (Cell Signaling Technology, 2118L), luciferase (ABclonal, requested), Ran (ABclonal, A0976), Hsp90 (Cell Signaling Technology, 4874). Images were acquired using LumiQuant AC600 (Acuronbio Technology Inc), quantification analysis was processed by ImageJ software. Trizol reagent (Sigma no. T9424) was used to extract total RNA from liver. RNAs were purified using RNeasy Mini kit (QIAGEN no. 4106). The quality and concentration of total RNA were checked on NanoDrop™ 2000/2000c Spectrophotometers (Thermo Fisher Scientific no. ND2000LAPTOP). Reverse transcription of total RNA was performed using a Applied Biosystems™ High-Capacity cDNA Reverse Transcription Kit (Thermo Fisher Scientific no. 4368813) and qPCR was conducted with SYBR Green Master Mix (Abclonal no. RK21203) on QuantStudio 6 Real-time PCR system (Thermo Fisher Scientific).

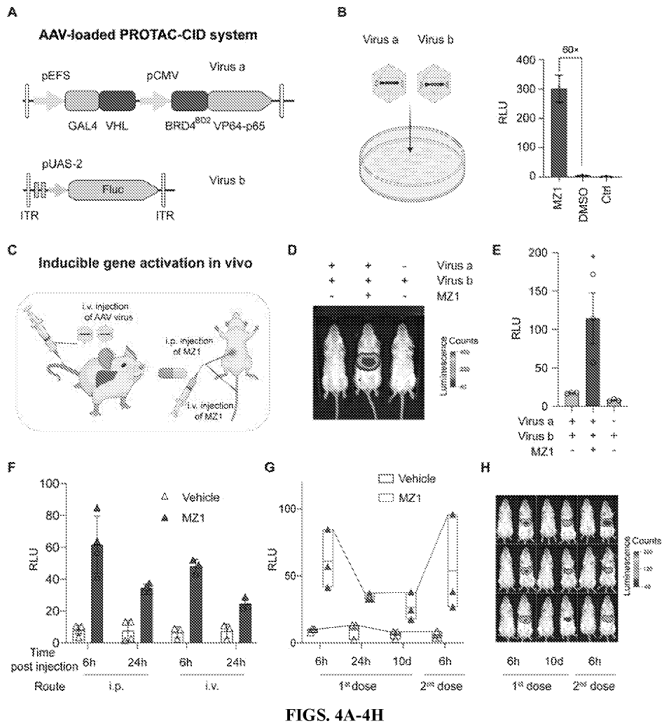

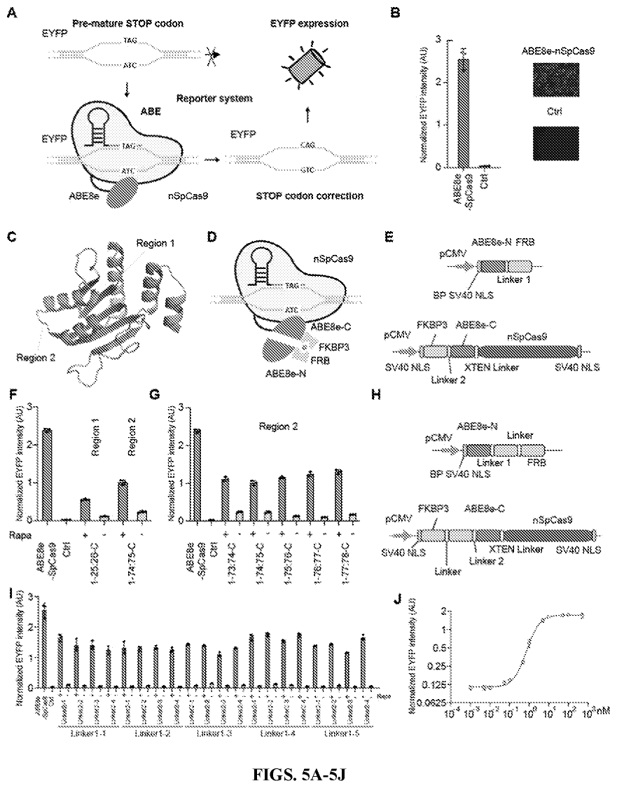

AAV production and Mouse for in vivo delivery. Mice were maintained and handled following laboratory animal treatments approved by the Institutional Animal Care and Use Committee (IACUC) of Baylor College of Medicine (BCM). FVB mice were purchased from the Jackson Laboratory. All mice were kept on 2920X Teklad Global Extruded Rodent Diet (Soy Protein-Free; Harlan Laboratories). 3-5 mice were housed in each cage in a 12 h light/12 h dark (LD, 7 am light-on, 7 pm light-off) condition with free access to water and food for all experiments. High titer and purity AAV viruses were produced by Neuroconnectivity Core of Baylor College of Medicine with 10 plates scale. These AAV viruses were then titered by real-time qPCR. 8-week-old FVB female mice were infected with either Virus a or Virus a and Virus b (FIGS. 4C-4H) at a dose of 2×10¹⁰ genome copies (GC) per mouse in saline (100 μL) via tail vein injection. 25 days after the virus injection, all mice were administrated with MZ1 at the concentration of 10 mg/kg through intraperitoneal injection (i.p.). To compare the route of administration, the inventors treated the mice with 50 mg/kg MZ1 by i.p. or 10 mg/kg by intravenous injection (i.v.). After ten days, to measure the repeatable activation, the inventors treated mice with 50 mg/kg MZ1 i.p. and observed the luciferase bioluminescence.

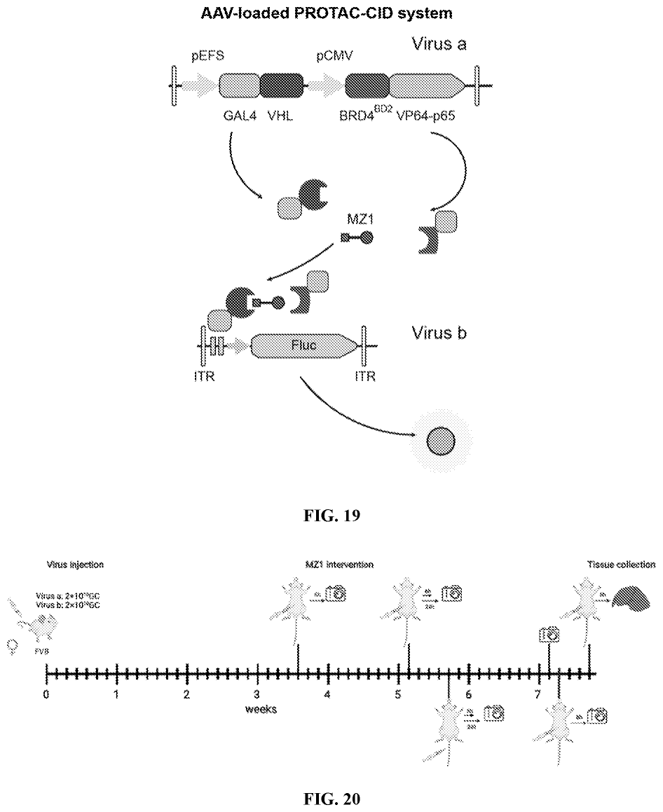

AAV infection on HEK293T cells. Before infection, HEK293T cells were seeded into 96 well plates (Corning no. 3610). When the confluency reaching 50%, 1 μL of each purified AAV virus (Virus a and Virus b in FIGS. 4 and 19) were added into the supernatant of HEK293T cells. 100 nM of MZ1 or DMSO was added into the supernatant. Three days post-infection, cells with 80 μL medium were treated with 40 μL lysis buffer (500 mM DTT, 10 mM coenzyme A, 100 mM ATP, 80 mg/mL D-luciferin, Triton lysis buffer (0.1082 M Tris-HCl, 0.0419 M Tris-Base, 75 mM NaCl, 3 mM MgCl₂)). To lysis the cells Plate was shaken by plate reader Infinite M200 (TECAN) with 20 seconds in “Orbital” mode with frequency of 432 rpm and Amplitude of 1 mm. Relative light units of luminescence was recorded by plate reader Infinite M200 (TECAN) with 1000 ms integration time using Megellan v1.7 (TECAN).

IVIS imaging system and quantification. Luciferase fluorescence intensity was measured by the IVIS imaging system (PerkinElmer). Mice were anesthetized with a mixture of isoflurane and oxygen. and then intraperitoneally (i.p.) injected with D-luciferin (15 mg/ml, GoldBio no. LUCNA-100). 5 mins after the D-luciferin injection, mice were imaged with IVIS imaging system. Quantitative analysis of imaging signals (luminescence counts) was processed by Living Imaging software (PerkinElmer).

Statistical Analysis. The number of independent experiments performed in parallel was represented by n in the figure legend. Two tailed Student's t-test was used for comparison shown in the figure legend. *P<0.05. No statistical methods were used to predetermine sample size. Most data in this research are represented by bar graphs with the mean and individual points. Unless otherwise indicated, representative images are from three biologically independent repeats. No data was excluded for analysis. For in vivo experiments, different biological repeats represent different mice. The inventors calculated the means and standard deviation (SD) with N-3 biological repeats unless stated otherwise. Prism 9 (GraphPad) was used to generate the bar plots and heatmap.

TABLE 4
Nonlinear regression analysis for calculating
EC50 of PROTAC-CID tools

| Description | Number |
|---|---|
| ABA | |
| Best-fit values | |
| Bottom | 1.587 |
| Hillslope | 2.083 |
| Top | 79.43 |
| EC₅₀ | 762.6 |
| logEC₅₀ | 2.882 |
| Span | 77.84 |
| Goodness of Fit | |
| Degrees of Freedom | 17 |
| R squared | 0.9724 |
| Sum of Squares | 631.1 |
| Sy.x | 6.093 |
| Constraints | |
| EC₅₀ | EC₅₀ > 0 |
| dTAG^{V}-1 | |
| Best-fit values | |
| Bottom | 0.9420 |
| Hillslope | 1.937 |
| Top | 56.67 |
| EC₅₀ | 227.8 |
| logEC₅₀ | 2.358 |
| Span | 55.73 |
| Goodness of Fit | |
| Degrees of Freedom | 26 |
| R squared | 0.9697 |
| Sum of Squares | 496.3 |
| Sy.x | 4.369 |
| Constraints | |
| EC₅₀ | EC₅₀ > 0 |
| dTAG-13 | |
| Best-fit values | |
| Bottom | 2.581 |
| Hillslope | 3.384 |
| Top | 138.3 |
| EC₅₀ | 52.98 |
| logEC₅₀ | 1.724 |
| Span | 135.7 |
| Goodness of Fit | |
| Degrees of Freedom | 23 |
| R squared | 0.9579 |
| Sum of Squares | 4269 |
| Sy.x | 13.62 |
| Constraints | |
| EC50 | EC50 > 0 |
| dTRIM24 | |
| Best-fit values | |
| Bottom | 0.5037 |
| Hillslope | 1.463 |
| Top | 1431 |
| EC₅₀ | 6347 |
| logEC₅₀ | 3.803 |
| Span | 1430 |
| Goodness of Fit | |
| Degrees of Freedom | 26 |
| R squared | 0.9968 |
| Sum of Squares | 2974 |
| Sy.x | 10.69 |
| Constraints | |
| EC₅₀ | EC₅₀ > 0 |
| Rapamycin | |
| Best-fit values | |
| Bottom | 6.692 |
| Hillslope | 1.871 |
| Top | 490.6 |
| EC₅₀ | 6.322 |
| logEC₅₀ | 0.8008 |
| Span | 483.9 |
| Goodness of Fit | |
| Degrees of Freedom | 29 |
| R squared | 0.9914 |
| Sum of Squares | 13564 |
| Sy.x | 21.63 |
| Constraints | |
| EC₅₀ | EC₅₀ > 0 |
| MZ1 | |
| Best-fit values | |
| Bottom | 1.416 |
| Hillslope | 1.728 |
| Top | 321 |
| EC₅₀ | 32.39 |
| logEC₅₀ | 1.51 |
| Span | 319.6 |
| Goodness of Fit | |
| Degrees of Freedom | 29 |
| R squared | 0.9781 |
| Sum of Squares | 13560 |
| Sy.x | 21.62 |
| Constraints | |
| EC₅₀ | EC₅₀ > 0 |

Step-by-step fluorescence protein activation assay protocol. Reagents:
1. PEI Max (Polysciences no. 24765-1)
2. DMEM, high glucose, GlutaMAX™ Supplement, pyruvate (Thermo Fisher Scientific no. 10569044)
3. Penicillin-Streptomycin (10,000 U/mL) (Thermo Fisher Scientific no. 15140122)
4. Fetal Bovine Serum 500ML (FBS) (Thermo Fisher Scientific no. 10437028)
5. TrypLE™ Express Enzyme (1×), phenol red (Thermo Fisher Scientific no. 12605068)
6. PBS pH=7.4 (Thermo Fisher Scientific no. 10010023)
7. Sony sorting chip 100 μM for MA900 (SONY no. LE-C3210)
8. Automatic Setup Beads kit (SONY no. LE-B3001)
9. DMSO (Sigma, no. D8418).

### Procedure:

1. Preparation of PEI Max solution. 50 mg PEI Max is dissolved in 45 mL Mill-Q Water. pH is adjusted to 7.1 by adding 10 M NaOH dropwise. Using Mill-Q water to adjust the volume to 50 mL. Filter the solution with 0.45 μM pore size Membrane filter (Millipore no. HAWP03700). Allocate the PEI Max solution to 1 mL and store at −20 degrees for use (avoiding multiple freeze-thaw cycles). Before using, PEI Max solution is heated with 65 degrees for 2 minutes.
2. Cell culture. HEK293T cells are seeded into 96 well plates (Corning no. 3598), 12-24 hour early before transfection when cell confluence achieves 50%. (Caution: HEK293T cells should be divided every two days to avoid overcrowding).
3. Transfection. 4.5 μL PEI Max solution and 300 μL DMEM are mixed (Caution: Do not use DMEM medium with FBS, FBS would interfere with transfection). Shake gently to mix. 180 ng of pUAS-1-EYFP, 180 ng of pHef1a-BFP, 180 ng of plasmids encoding GAL4 fused protein and 180 ng of plasmids encoding VPR fused protein were mixed into 1.7 mL tube (Caution: plasmids should be prepared freshly with good quality and preservation. Bad plasmid preservation would affect the gene activation ability dramatically). Mix the PEI-DMEM solution with plasmids and incubate at room temperature for 30 minutes. Add ⅓ volume of the mixture to one well of 96 well plates gently. Return the 96 well plates for culture.
4. Gene induction. 12 hours after transfection, cell culture medium is replaced with DMEM with 10% FBS and 100 U Penicillin-Streptomycin. DMSO dissolved inducer or DMSO as control is added according to the experiments.
5. Flow cytometry sample collection. 2 d after transfection, remove the cultured medium and ˜ 50 μL trypLE is added into each well. After 3 minutes of digestion (Caution: cells should be thoroughly digested to avoid cell clusters to block the flow cytometry, but too long time of digestion will be harmful for the cells). 100 μL of DMEM with 10% FBS are added into wells to inactivate the trypLE. Cell-containing medium is moved to 1.7 mL tube. Centrifuge the medium with 3000 g at 5 minutes. Remove the supernatant and 200 μL PBS is added to make the single cell solution. Transfer the cells to 12×75 mm flow tubes.
6. Flow cytometry data collection. Before running, change the sorting chip on MA900 every 24 hours and check the MA900 flow cytometry with setup beads. Collect the cells as the parameter described on Methods. Clean the MA900 flow cytometry with 10% bleach and water after using.

### Example 2—Establish PROTAC-CIDs for Inducible Gene Expression in Mammalian Cells

The inventors first fused the GAL4 DNA binding domain or the VP64-p65-Rta (VPR) transactivation domain (30) to each of the PROTAC interacting protein partners. The dimerization of target proteins and E3 ubiquitin ligases induced by PROTACs will bring GAL4 and VPR into proximity to drive the downstream reporter gene expression (enhanced yellow fluorescence protein, EYFP) (FIG. 1A). Out of nine commercially available PROTACs (Table 5), dTRIM24 (31), dTAG^{V}-1 (32), AT1 (33), and MZ1 (34) were used to conjugate the E3 ubiquitin ligase (VHL) with various target proteins (TRIM24, FKBP12^{F36V}, or BRD4, respectively); while TL13-12 (35), TL13-112 (35), dTAG-13 (36), dBRD9 (37), and ZXH3-26 (38) were implemented to dimerize the E3 ubiquitin ligase (CRBN) with different target proteins truncated ALK (tALK), BRD9, FKBP12^{F36V} or BRD4, respectively. The inventors employed the full-length PROTAC target proteins and the E3 ubiquitin ligases for initial tests, except that the small-molecule binding kinase domain tALK was truncated from the membrane target protein ALK based on its crystal structure to facilitate nucleus translocation (39). The inventors co-transfected plasmids encoding these fusion genes into HEK293T cells, together with the reporter plasmid encoding EYFP driven by the GAL4 cognate pUAS promoters (FIGS. 11A and 11B). After 2 days of induction, all the nine PROTAC-CID systems induced EYFP expression compared with the control samples using DMSO (FIG. 1B). Notably, several PROTACs, dTRIM24, dTAG-13, and MZ1, showed more than a 100-fold increase in EYFP expression, which is more efficient than the commonly used ABA-based CID system (16) for gene activation (FIG. 1B). Molecular glue Lenalidomide was previously identified to degrade IKAROS Family Zinc Finger 1 (IKZF1) or IKZF3 by recruiting CRBN (40). To compare the activity of PROTACs with Lenalidomide for inducible gene activation, the inventors designed a similar gene activation platform by fusing CRBN with VPR and GAL4 with either IKZF1 or IKZF3. However, Lenalidomide was significantly less efficient than the dTAG-13 PROTAC-CID system that also uses the CRBN and VPR fusion (FIGS. 11A-11E). These results highlight the advantage of modular PROTACs for CID-based gene activation.

TABLE 5
Protein partners and fusion strategies in FIG. 1B

| | Fusion protein 1 | | | |
|---|---|---|---|---|
| | (E3 ubiquitin | Fusion protein 2 | Concentration | |
| | ligases are | (Target proteins | of the small | |
| | underlined) | are underlined) | molecules | |
| PROTACs | | | | |
| dTRIM24 | GAL4-VHL | TRIM24-VPR | 5 | μM |
| dTAG^{V}-1 | VHL-VPR | GAL4-FKBP12^{F36V} | 5 | μM |
| AT1 | GAL4-VHL | BRD4-VPR | 1 | μM |
| MZ1 | GAL4-VHL | BRD4-VPR | 100 | nM |
| TL 13-12 | CRBN-VPR | GAL4-tALK | 1 | μM |
| TL13-112 | CRBN-VPR | GAL4-tALK | 1 | μM |
| dTAG-13 | CRBN-VPR | GAL4-FKBP12^{F36V} | 100 | nM |
| dBRD9 | CRBN-VPR | GAL4-BRD9 | 1 | μM |
| ZXH3-26 | CRBN-VPR | GAL4-BRD4 | 1 | μM |
| CID inducers | | | | |
| ABA | GAL4-ABI | PYL-VPR | 250 | μM |
| Rapamycin | GAL4-FKBP3 | FRB-VPR | 1 | μM |
| Rapamycin | GAL4-FKBP12 | FRB-VPR | 10 | nM |

The relatively large size of some target proteins, e.g., BRD9, BRD4, and TRIM24 (67, 80, and 117 kDa, respectively), could impose conformational constraints and limit the accessibility of PROTACs to form stable heterodimers (33). The bromodomains (BDs) alone in TRIM24 (31) and BRD9 (37) can bind to the dTRIM24 and dBRD9 PROTACs; while the BD1 and BD2 in BRD4 (33) are capable of binding MZ1, and AT1 PROTACs. Fusion proteins of VPR with the BDs from BRD4 and TRIM24 (BRD4^{BD2}-VPR (SEQ ID NO: 7), and TRIM24BD-VPR (SEQ ID NO: 4)) showed significant enhancement in EYFP activation when co-transfected with GAL4-VHL (SEQ ID NO: 3) (FIGS. 1C and 1D). Compared to the DMSO-treated controls, TRIM24BD-VPR (SEQ ID NO: 4) achieved a 592-fold increase in EYFP expression, and BRD4^{BD2}-VPR (SEQ ID NO: 7) displayed 441-fold EYFP induction, which exceeded that of rapamycin-based gene activation (355-fold) using FRB and FKBP12 (FIGS. 1B, 1C, and 1D). The truncated GAL4-BRD9BD (SEQ ID NO: 13) also displayed increased EYFP expression compared with GAL4-BRD9 (SEQ ID NO: 12) (FIG. 1E). Thus, these results indicated that truncation of the target proteins could further enhance and develop robust PROTAC-CID platforms, possibly due to the enhanced PROTAC accessibility to the interaction domains. The inventors also tested two truncated CRBN variants without ubiquitin ligase function by deleting the 7-α-helical bundle domain to eliminate the interaction between CRBN with Damage Specific DNA Binding Protein 1. Both of these variants still showed robust gene activation ability, demonstrating the possibility to decrease the undesired effects raised by overexpressing E3 ubiquitin ligases (FIG. 12).

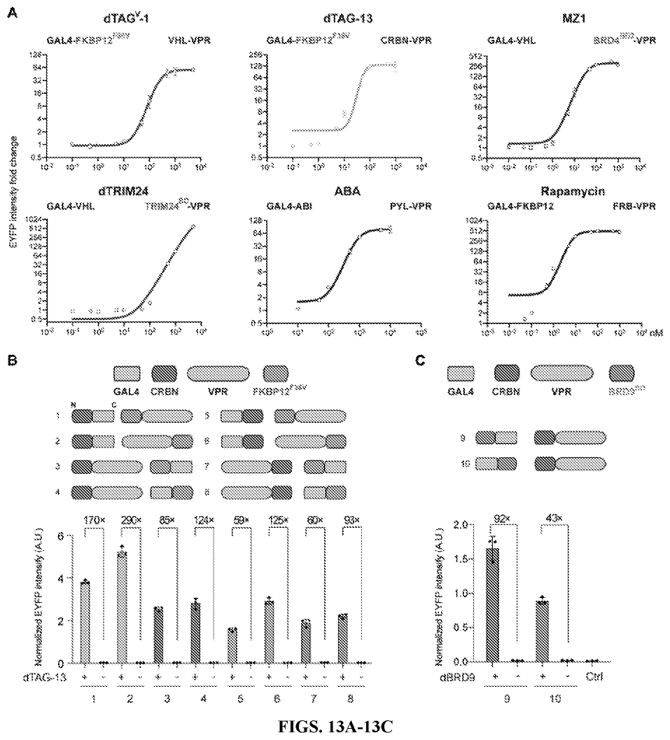

To characterize the sensitivity of the engineered PROTAC-CID systems, the inventors profiled the dose-response for several PROTACs. dTAG-13 and MZ1 showed low EC₅₀ values of 53 nM and 32 nM, which is slightly higher than that of rapamycin (6 nM). dTAG^{V}-1 had higher EC₅₀ values of 228 nM, although it was still more sensitive than ABA (EC₅₀ 763 nM) for gene activation (FIG. 13A). Additionally, although dTRIM24 achieved more than 500-fold change gene activation activity at 5 μM, high EC₅₀ (6.3 μM) was observed and dTRIM24 displayed weak inducible gene activation ability below 1 μM with less than 100-fold change in EYFP expression (FIG. 13A). The possible reason would be due to the poor cell permeability (41), which restricts the intracellular delivery of dTRIM24. To validate the modularity of the protein fusions in the PROTAC-CID systems, the inventors tested different domain organizations in constructing the PROTAC target proteins, E3 ubiquitin ligases, GAL4, and VPR for EYFP activations for both the dTAG-13 and dBRD9 systems. All protein fusions successfully activated EYFP expressions to different levels (43- to 290-fold). These studies corroborated the feasibility and robustness of the PROTAC-CID systems in transgene activation (FIGS. 13B and 13C). Thus, the inventors established scalable PROTAC-CID systems for high-fold and sensitive inducible gene expression in mammalian cells.

### Example 3—Multiplex and Gradient Gene Regulation Enabled by PROTAC-CID Systems

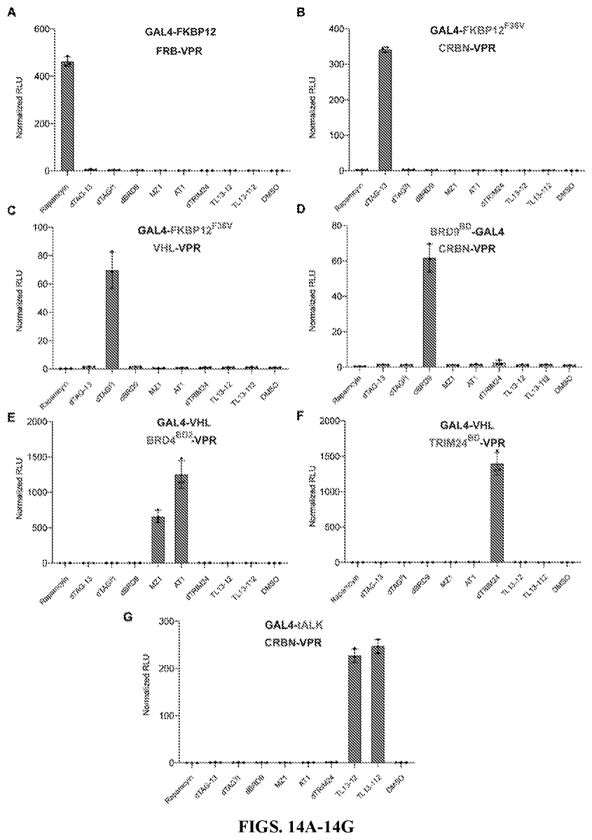

Since several PROTACs interact with the same protein partners (Table 5), the inventors tested the orthogonality of these PROTAC-CID systems in triggering gene activation with cognate or non-cognate protein pairs. Each small molecule (including eight high-fold gene activation PROTACs and rapamycin) was added to the HEK293T cells transfected with plasmids for all different combinations of protein partners (seven different pairs in total). The successful dimerization of two protein pairs will drive the Firefly luciferase gene (Fluc) expression for high throughput readouts. High inductions (62- to 1396-fold) of Fluc were only observed under the correct cognate combinations (FIGS. 2A and 14), e.g., dTAG-13 only activated Fluc expression in the cells transfected with plasmids containing GAL4-FKBP12^{F36V} (SEQ ID NO: 1) and CRBN-VPR (SEQ ID NO: 2) coding regions, while not in other samples with non-cognate protein partners (FIGS. 2A and 14). MZ1/AT1 and TL13-12/TL-112 interact with the same protein pairs as expected. These results demonstrated the great orthogonality of the PROTAC-CID systems for high-level inducible gene expression, paving the road for multiplexing gene regulations.

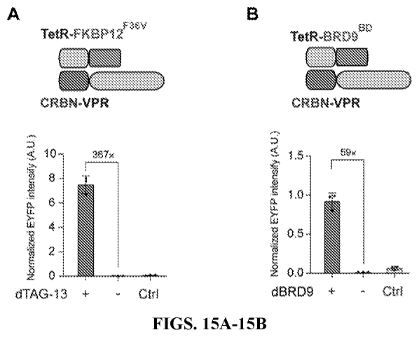

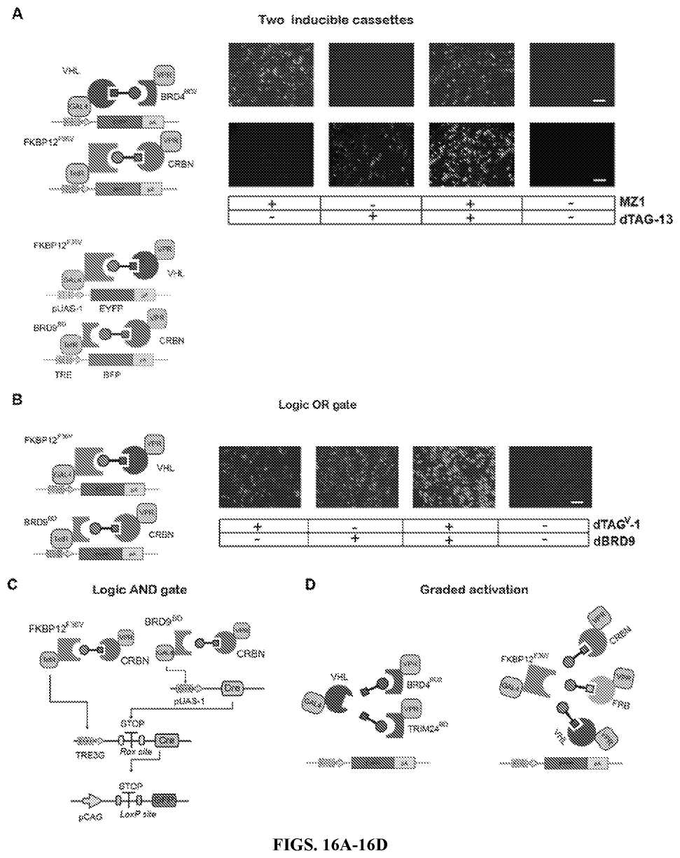

The inventors next tested the feasibility of dual PROTAC-based inducible gene cassettes (FIG. 15). In the first cassette, GAL4 was fused with FKBP12^{F36V}, and VHL was ligated with VPR to drive EYFP expression in response to dTAG^{V}-1. In the second cassette, TetR was fused with BRD9BD, and CRBN was connected with VPR to drive blue fluorescence protein (BFP) expression in the presence of dBRD9. The inventors observed EYFP or BFP expression only in the presence of its corresponding inducer dTAG^{V}-1 or dBRD9, while activation of both EYFP and BFP could be achieved by administrating dTAG^{V}-1 and dBRD9 simultaneously (FIG. 2B). Similarly, the MZ1 and dTAG-13 PROTAC-CID systems showed single gene activation with one PROTAC small molecule and dual-gene activation using both PROTACs (FIG. 16A). As biological computation relies on the protein or DNA to execute Boolean logic gate operation in living organisms for cell discrimination and disease diagnosis (3, 11, 42), the inventors next explored the possibility of PROTAC-CID enabled logic gate biological computation. By placing EYFP under control of the TRE (Tetracycline response element) and pUAS-1 promoter, the inventors observed strong EYFP expression by using one or two PROTACs, achieving clear logic OR gate responses (FIGS. 2C and 16B). To develop a more sophisticated logic AND gate, the inventors took advantage of two orthogonal site-specific DNA recombinases (Cre and Dre) for biological computation (43). Pre-stop transcription polyA signal (STOP) flanked by Cre or Dre DNA recombination site (LoxP-STOP-LoxP or Rox-STOP-Rox) was put upstream of the gene of interest to prevent gene expression. Two PROTAC-CID gene activation systems (dTAG-13 and dBRD9) were designed to drive the Cre and Dre expression, respectively. The Cre recombinase gene is placed downstream of the Rox-STOP-Rox DNA sequence as a “roadblock”, which can only be removed by the induced Dre recombinase. In this gene circuit, only in the presence of both dTAG-13 and dBRD9 inducers, Dre and Cre can be expressed to remove their respective “STOP” signals, resulting in the eventual GFP expression as a clear logic AND readout (FIGS. 2D and 16C).

One of the limitations of a single inducer-controlled gene expression system is the existence of only one input, which restricts the programmability of gene activation (24). The inventors hypothesized that a multi-state transcriptional control system with different gradient gene activation could be achieved by combining different PROTAC-CID systems. Notably, some of the PROTACs can bind with the same E3 ubiquitin ligases, e.g., dTRIM24 and MZ1 both conjugate VHL, but bind to different target proteins (TRIM24 and BRD4). When HEK293T cells are transfected with GAL4-VHL (SEQ ID NO: 3), TRIM24^{BD}-VPR (SEQ ID NO: 4), BRD4^{BD2}-VPR (SEQ ID NO: 7), and the reporter plasmid, the inventors observed three grades of EYFP intensity, 13-fold, 37-fold, and 120-fold, with MZ1, dTRIM24, and MZ1 plus dTRIM24, respectively. Likewise, rapamycin, dTAG-13, and dTAG^{V}-1 share the same target protein FKBP12^{F36V} but recruit three different cognate partners (FRB, CRBN, and VHL) with various affinities. The inventors also achieved three grades of activation by rapamycin, dTAG-13, and dTAG^{V}-1 (FIGS. 2E and 16D) in HEK293T cells transfected with all related constructs. Thus, the PROTAC-CID systems enable graded gene regulation.

### Example 4—Application of PROTAC-CIDs to Regulate Genome Editing

Site-specific Cre DNA recombinases (44), base editors (BEs) (45), and prime editors (PE) (46) have revolutionized the ability to manipulate genomes in living cells. Inducible expression of these toolkits could reduce the exposure time of the host genome to genome-editing tools and increase the safety of targeted genome modifications. To test whether PROTAC-CIDs can be used to induce Cre-based site-specific DNA recombination, the inventors designed a “two-layer” genetic circuit and transfected plasmids encoding the dTAG-13 or dBRD9 PROTAC-CID system to drive the Cre expression in HEK293T cells. LoxP-STOP-LoxP cassette was placed upstream of gfp gene, where Cre protein can be recruited to remove the pre-mature STOP signal for Cre-mediated GFP expression. The inventors observed a strong GFP signal in the presence of 100 nM dTAG-13 or 1 μM dBRD9 (FIGS. 3A and 17). However, leaky expression of GFP was observed in both cases without PROTACs, which was similar to previous reports with tetracycline and rapamycin inducible systems (47-49).

Although most biological processes are analog signals that start from a basal level to a higher level continuously (50), digital signals that can switch from zero to one sharply shown as ON and OFF states are critical for controlling the expression of certain genes, such as genome modifying agents. To address if the PROTAC-CID system could tightly control Cre expression as digital outputs, the inventors designed a “three-layer” genetic circuit by adding the orthogonal DNA recombinase, Dre. To eliminate the leaky expression at the un-stimulated state, the inventors put a Rox-STOP-Rox site between the TRE3G promoter and Cre DNA coding region. Upon adding dTAG-13, the PROTAC-CID system induces Dre expression to remove the STOP signal in front of the Cre gene. Downstream Cre expression then removes the “STOP” between the LoxP sites and leads to the eventual expression of GFP (FIG. 3B). With the “three-layer” gene expression control circuit, the inventors observed robust GFP expression only in the presence of dTAG-13, indicating that the PROTAC-CID system could be combined with other synthetic genetic circuits to enable tight, digital gene regulation (FIG. 3B).

Next, the inventors aimed to apply the PROTAC-CID systems to control CRISPR base editors (BEs) expression in mammalian cells. Two main classes of DNA-modifying BEs have been developed to date, including cytosine BEs (CBEs) and adenine BEs (ABEs), converting C·G-to-T·A and A·T-to-G·C, respectively. However, BEs have been reported with significant off-targets in Cas9-dependent and/or independent manners in both genomic and transcriptomic levels (45). To enable PROTAC-based inducible base editing, the inventors first integrated the previously developed CBE A3G5.13 (51) with the dTAG-13 PROTAC-CID system. The inventors observed efficient 30-50% C-to-T editing across three different genomic sites in the presence of 100 nM dTAG-13 and only low levels of editing (4-11%) were detected without PROTACs (FIG. 3C). However, when inducible ABE8e (52) was tested by using the PROTAC-CID system, a relatively high background A-to-G editing (27.0%˜ 59.7% in two genomic sites) was observed without dTAG-13 induction. Since the engineered ABE8e was highly active, the inventors speculated that even the low level of the leaky ABE expression could result in significant genome editing outcomes. Therefore, the inventors coupled the Cre DNA recombinase with the PROTAC-CID system to decrease the basal level of ABE8e activity. By placing the LoxP-STOP-LoxP cassette between the TRE3G promoter and the region encoding the ABE8e, the inventors observed high A-to-G editing (30.0% to 49.3%) in the presence of dTAG-13, while significantly reduced editing efficiency without induction (7.3% to 15.3%), demonstrating the feasibility of PROTAC-CID enabled genetic circuits for low-basal level induction of genome editors (FIGS. 3D and 3E). Finally, the inventors sought to test whether the PROTAC-CID system can be used to develop inducible Primer editors (PEs) (46). The inventors engineered a mutated Cre gene with 2-bp micro-deletion, which has no site-specific DNA recombinase activity due to frameshift. Successful insertion of the missing 2-bp by PE will restore Cre activity to remove the STOP signal for GFP expression, thus indicating prime editing repair efficiency. While constitutionally expressed PE2 elicited higher GFP intensity, the dTAG-13 induced a more than 25-fold increase in GFP expression without any leaky baseline editing, showing that the PE system can also be integrated into the “three-layer” genetic circuit and enable tight gene regulation (FIGS. 3F and 18). When the inventors applied the inducible PE system to insert a His₆ Tag into an endogenous HEK3 locus in the HEK293T cells, more than 20% prime editing efficiency in the presence of dTAG-13 was observed with a high TRE3G-driven PE2 plasmid dose (120 ng per well) (FIGS. 3G and 3H), although 5.8% of editing was in the control with no PROTAC added. When a lower TRE3G-driven PE2 plasmid dose (50 ng per well) was used, nearly unobservable leaky His₆ Tag insertion in the unstimulated group was found (FIGS. 3G and 3H). These results demonstrate that the PROTAC-CID system can be used for inducible base- and prime-editing.

### Example 5—In Vivo PROTAC-CID Based Inducible Gene Activation Through AAV Delivery

Gene therapy has revolutionized the treatment of previously untreatable genetic diseases. Coupling PROTAC-CID with AAV could allow precise dosage or spatiotemporal control of gene expression in vivo, potentially valuable for toxicity management or personalized gene therapy. To test the PROTAC-CID system for in vivo applications, the inventors designed a compact PROTAC-CID system in AAV vectors (FIGS. 4A and 19). Since the gene fragments encoding BRD4^{BD2} and VHL are smaller than 700 bp and MZ1 displayed a low EC₅₀ (FIG. 13), the inventors selected the MZ1 PROTAC-CID systems by placing the GAL4-VHL (SEQ ID NO: 3) and BRD4^{BD2}-VP64-p65 (SEQ ID NO: 34) in the AAV vector (FIG. 4A Virus a). The inventors constructed another AAV vector expressing Firefly luciferase (Fluc) gene driven by the pUAS-2 promoter (FIG. 4A Virus b). After co-infecting the HEK293T cells in vitro, the inventors treated the infected cells with 100 nM MZ1 and observed a more than 60-fold increase in luciferase bioluminescence intensity (FIG. 4B).

To validate the ability of PROTACs for in vivo inducible gene activation via AAV (FIG. 4C), the inventors intravenously injected 8-week-old adult FVB mice with both Virus a and Virus b with AAV serotype 8 (FIG. 20). 25 days after the AAV injection, the inventors treated mice with 10 mg/kg MZ1 intraperitoneally and performed the bioluminescence imaging 6 h post-MZ1 treatment (FIG. 4D). The inventors observed a significant increase (˜7-fold) of the luciferase expression in the liver compared with the group without the MZ1 treatment (FIG. 4E). The basal bioluminescence intensity from the vehicle control group is comparable to the only Virus b treated group, demonstrating a low leaky expression in the absence of MZ1. Administrating MZ1 intraperitoneally or intravenously can both increase bioluminescence levels (7- to 8-fold), suggesting MZ1 is compatible with multiple administration routes (FIG. 4F).

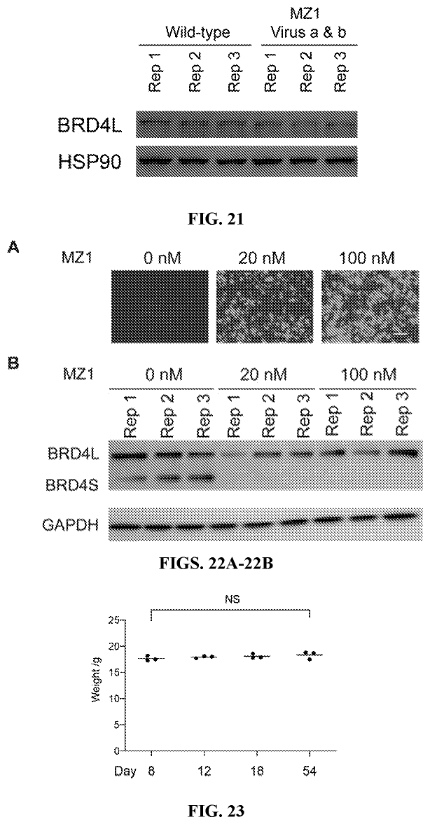

Distinct from CRISPR-based gene editing that a single dose of genome editors give a permanent modification for life-long benefits, gene therapy might require transient or reversible activation of transgene expression in the alignment of the daily rhythm (e.g., the rhythm of insulin) or according to disease progression. Therefore, the ability to modulate the transgene expression levels is desirable (53-56). To test the possibility of activating gene expression repeatably, the inventors provided a second MZ1 injection ten days after the first MZ1 treatment and observed an elevated expression of Fluc with a comparable gene activation level as the first administration, demonstrating the ability for repeatable and reversible induction of the transgene expression (FIGS. 4G and 4H). The inventors did not observe a significant change in the expression of BRD4 in mice liver tissue, suggesting that the dose of MZ1 was not sufficient to degrade the endogenous target protein in the healthy liver (FIGS. 4 and 21). Similarly, in cultured HEK293T cells, the inventors only observed the degradation of the short isoform of BRD4 (BRD4S), not the conventional long isoform BRD4 (BRD4L) in the presence of MZ1 (20 nM and 100 nM) (FIG. 22). The inventors observed no abnormality or body weight change after MZ1 administration, indicating the relative safety of MZ1 and the PROTAC-CID system (FIG. 23). In summary, the compact PROTAC-CID system in AAV enables inducible, repeated gene expression regulation in vivo.

### Example 6—Split Inducible ABEs

For rapid exploration of inducible ABE variants, the inventors first sought to develop a rapid fluorescence-based reporter system to quantify the ABE efficiency as reported before. The inventors selected target region in eyfp gene with NGG PAM where a CAG codon within the editing window was mutated to TAG stop codon. ABEs convert A to G or T to C, thus allowing the conversion of stop codon to CAG following gRNA binding to untemplated strand. Additionally, there is no other bystander A in the editing window that would cause complex editing to affect the fluorescence expression. The inventors observed a high restoration of the EYFP fluorescence when transfecting HEK293T cells with plasmids encoding the ABE8e fused SpCas9 and guide RNA (FIGS. 5A and 5B). Next, the target range of base editors was restricted by the PAM where the prototypical SpCas9 recognizing target sites with NGG PAM (where N is A, C, G or T). A variant named SpG was identified with an expanded set of NGN PAM. Thus, to establish SpG compatible reporter system, the inventors identified another site in the eyfp gene where a stop codon was installed with NGT PAM. SpG fused ABE8e was observed with clear signal in both of NGG and NGN reporter displaying the PAM expansion ability of SpG fused base editors (FIGS. 8A and 8B).

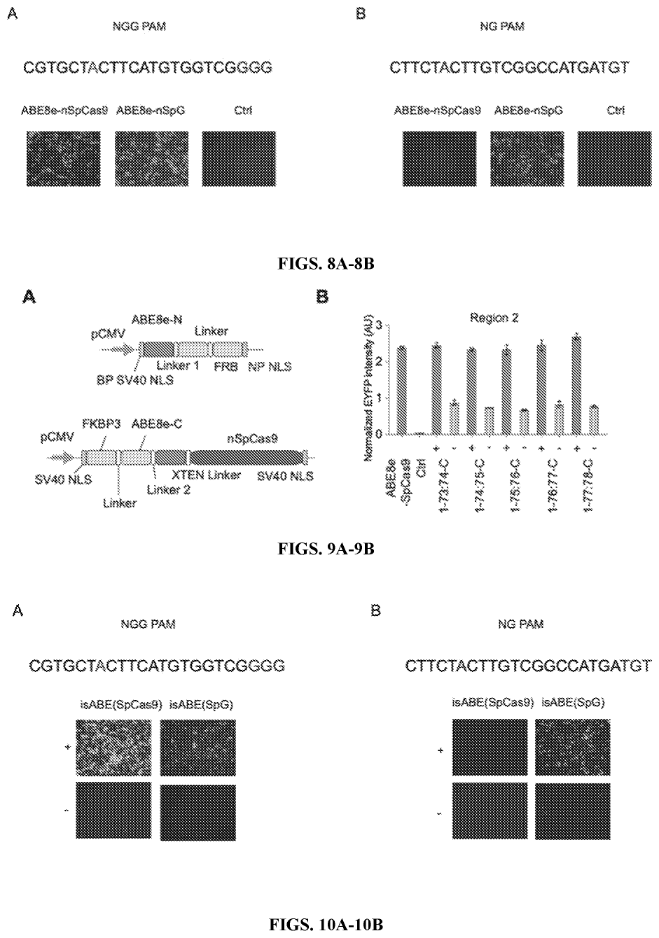

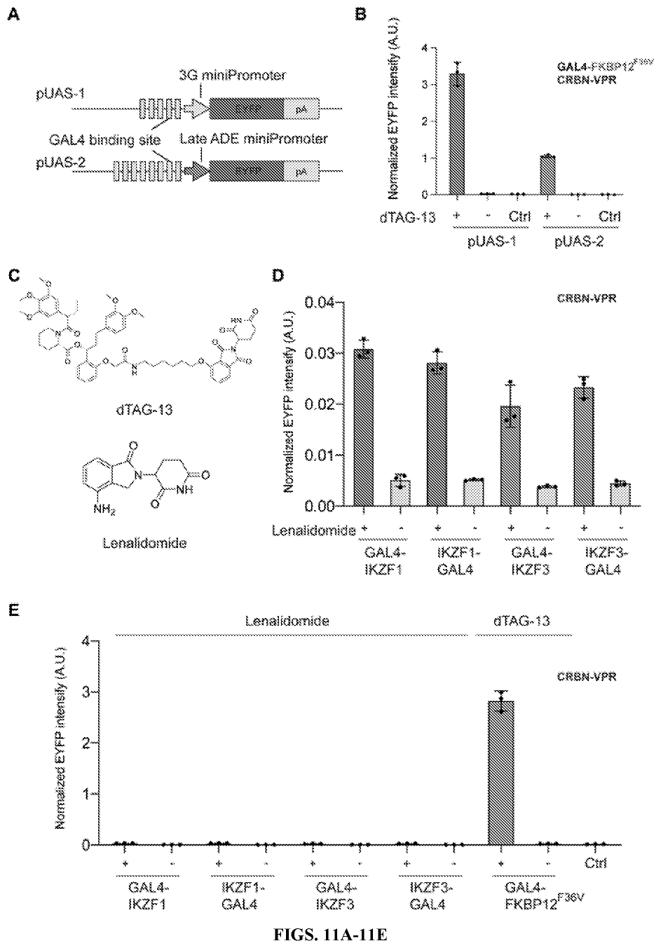

To develop a split adenosine base editor system, the inventors identified two potential split regions based on the crystal structure of ABE8e base editor in complex with guide RNA and target DNA. The inventors chose one residue site (25 and 74) in each region and fused the resulting N-terminal ABE8e fragment with FRB and C-terminal ABE8e fused to Cas9 nickase fragment with FKBP3, respectively. The split ABE constructs were tested by targeting the EYFP reporter system in HEK293T cells. Using flow cytometery, both strategies generated obvious EYFP signal. In addition, moderate levels of eYFP signal were detected in both sites. Since split site 74 gave a higher efficiency, the inventors chose region two for more detailed exploration. The inventors reasoned that the high basal level and low efficiency could be optimized by fusing with different linkers, changing the split sites, and varying the copy number of interacting domains. Firstly, the inventors tested all the residues in region 2 from 73 to 77. Interestingly, the split site in 76 generated a low leaky EYFP signal without the Rapamycin induction and the same level EYFP expression in the presence of Rapamycin. Additionally, an additional NLS signal was fused to the C-terminus of the FRB domain, which resulted in all five combinations of split ABE system yielding ˜two-fold increase of the EYFP expression with high basal level (FIGS. 9A and 9B). Next, the inventors selected the 76 splitting site and further fused two copies of FRB and FKBP3 domains with various linkers (FIGS. 5H and 5I). Notably, all the constructs showed ˜50% increased EYFP signal under the stimulation of Rapamycin and low leaky level. Thus, the inventors select the first one (linker1-1 and linker2-1) for the following research (refer as isABE system). The inventors further measured the dose response of the isABE system and found that isABE showed high sensitivity with EC₅₀ of 1.8 nM. Additionally, when SpCas9 was replaced with SpG, it also worked with isABE system (FIGS. 10A and 10B).

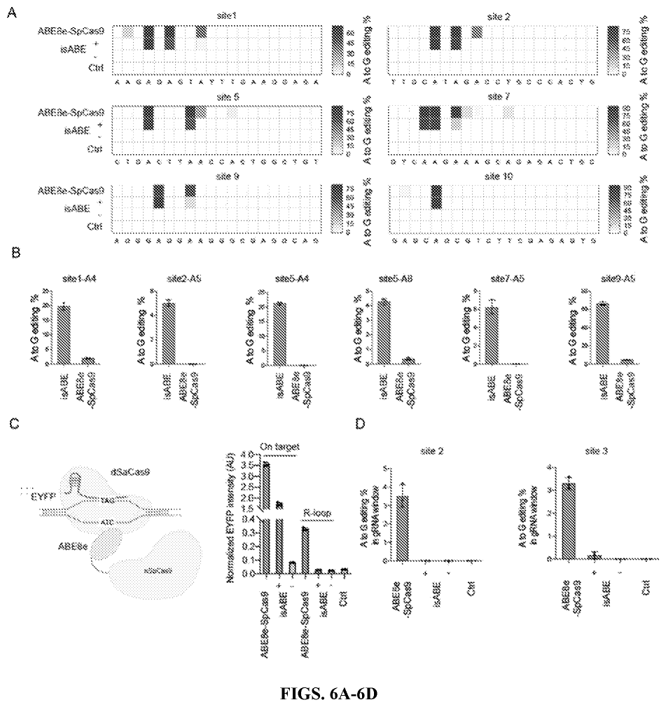

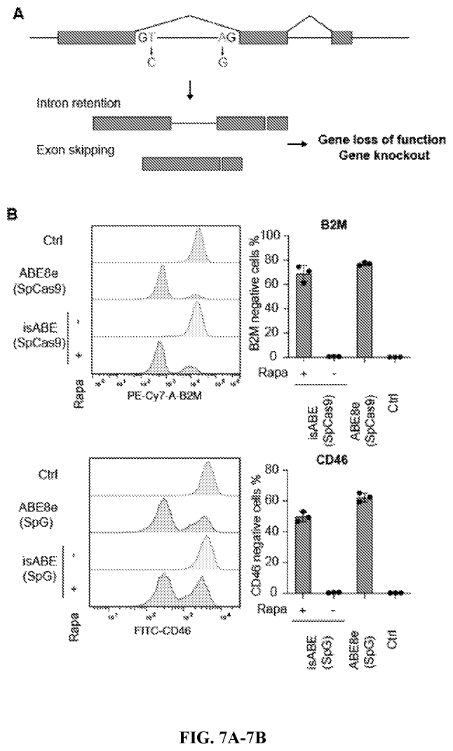

To explore the endogenous editing efficiency, the inventors chose six different sites and transfected the plasmids encoding the gRNA and the ABE system into HEK293T cells. In all of the six sites, the isABE system generated similar levels of A to G editing in the most efficient editing base of the window (FIG. 6A). Additionally, isABE generates low level at the bystander sites which can lead to accurate base editing. Shown as (FIG. 6B), the isABE can enable accurate single A editing in the editing window from 4-66% while in three sites, the ABE8e-SpCas9 can generate the single A editing and in another 3 sites displayed less 5% editind efficiency. Since almost all the genetic disease that needs to be repaired are with single nucleotide mutation in the editing window, the isABE system with high accurate editing capability provide a unique tool for highly efficient and accurate base editing. Base editors can mutate the single strand DNA to generate unwanted off-target editing. To evaluate the off-target effect, the inventors adopted R-loop based evaluation with artificial R-loop opened by an orthogonal dSaCas9. Firstly, the inventors created a reporter system where the dSaCas9 can open the pre-mature STOP codon region. The free ABE8e-fused nSpCas9 can mutate ssDNA in the R-loop although without the guidance of the guide RNA. The inventors observed a significant EYFP expression when expressing the ABE8e-nSpCas9 with more than 10-fold compared with control group. Interestingly, isABE did not show any increased EYFP signal in the presence of Rapamycin suggesting a very low random ssDNA editing of isABE system. Furthermore, the inventors chose two endogenous sites opened by dSaCas9 and found a similar result in which ABE8e-nSpCas9 can lead to more than 3% editing efficiency while the off-target effect of the isABE is undectable or around 0.2% (FIGS. 6C and 6D). Next, the inventors further applied the isABE system for inducible gene knockout by interrupting RNA splicing sites. The intron retention or exon skipping due to the wrong RNA splicing would lead to gene loss of function. The inventors chose two genes (B2M and CD46) and found that the isABE system can generate high level (more than 40%) of negative cells populations detected by flow cytometry (FIGS. 7A and 7B).

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
1. B. Z. Stanton, E. J. Chory, G. R. Crabtree, Chemically induced proximity in biology and medicine. Science 359, (2018).
2. A. Fegan, B. White, J. C. Carlson, C. R. Wagner, Chemically controlled protein assembly: techniques and applications. Chem Rev 110, 3315-3336 (2010).
3 T. Kitada, B. DiAndreth, B. Teague, R. Weiss, Programming gene and engineered-cell therapies with synthetic biology. Science 359, (2018).
4. M. G. Jaeger, G. E. Winter, Fast-acting chemical tools to delineate causality in transcriptional control. Mol Cell 81, 1617-1630 (2021).
5. M. Gossen, S. Freundlieb, G. Bender, G. Muller, W. Hillen, H. Bujard, Transcriptional activation by tetracyclines in mammalian cells. Science 268, 1766-1769 (1995).
6. A. T. Das, L. Tenenbaum, B. Berkhout, Tet-On Systems For Doxycycline-inducible Gene Expression. Curr Gene Ther 16, 156-167 (2016).
7. N. Alerasool, H. Leng, Z. Y. Lin, A. C. Gingras, M. Taipale, Identification and functional characterization of transcriptional activators in human cells. Mol Cell 82, 677-695 e677 (2022).
8. Y. Gao, X. Xiong, S. Wong, E. J. Charles, W. A. Lim, L. S. Qi, Complex transcriptional modulation with orthogonal and inducible dCas9 regulators. Nat Methods 13, 1043-1049 (2016).
9. M. M. Chang, L. Gaidukov, G. Jung, W. A. Tseng, J. J. Scarcelli, R. Cornell, J. K. Marshall, J. L. Lyles, P. Sakorafas, A. A. Chu, K. Cote, B. Tzvetkova, S. Dolatshahi, M. Sumit, B. C. Mulukutla, D. A. Lauffenburger, B. Figueroa, Jr., N. M. Summers, T. K. Lu, R. Weiss, Small-molecule control of antibody N-glycosylation in engineered mammalian cells. Nat Chem Biol 15, 730-736 (2019).
10. W. Deng, J. A. Bates, H. Wei, M. D. Bartoschek, B. Conradt, H. Leonhardt, Tunable light and drug induced depletion of target proteins. Nat Commun 11, 304 (2020).
11. C. Y. Wu, K. T. Roybal, E. M. Puchner, J. Onuffer, W. A. Lim, Remote control of therapeutic T cells through a small molecule-gated chimeric receptor. Science 350, aab4077 (2015).
12. H. Wang, X. Xu, C. M. Nguyen, Y. Liu, Y. Gao, X. Lin, T. Daley, N. H. Kipniss, M. La Russa, L. S. Qi, CRISPR-Mediated Programmable 3D Genome Positioning and Nuclear Organization. Cell 175, 1405-1417 e1414 (2018).
13. Y. Gao, M. Han, S. Shang, H. Wang, L. S. Qi, Interrogation of the dynamic properties of higher-order heterochromatin using CRISPR-dCas9. Mol Cell 81, 4287-4299 e4285 (2021).
14. E. J. Brown, S. L. Schreiber, A Signaling Pathway to Translational Control. Cell 86, 517-520 (1996).
15. E. J. Brown, M. W. Albers, T. B. Shin, K. Ichikawa, C. T. Keith, W. A. S. Lane, S. L. Schreiber, A mammalian protein targeted by G1-arresting rapamycié receptor complex. Nature 369, 756-758 (1994).
16. F. S. Liang, W. Q. Ho, G. R. Crabtree, Engineering the ABA plant stress pathway for regulation of induced proximity. Sci Signal 4, rs2 (2011).
17. T. Miyamoto, R. DeRose, A. Suarez, T. Ueno, M. Chen, T. P. Sun, M. J. Wolfgang, C. Mukherjee, D. J. Meyers, T. Inoue, Rapid and orthogonal logic gating with a gibberellin-induced dimerization system. Nat Chem Biol 8, 465-470 (2012).
18. M. J. Ziegler, K. Yserentant, V. Dunsing, V. Middel, A. J. Gralak, K. Pakari, J. Bargstedt, C. Kern, A. Petrich, S. Chiantia, U. Strahle, D. P. Herten, R. Wombacher, Mandipropamid as a chemical inducer of proximity for in vivo applications. Nat Chem Biol 18, 64-69 (2022).
19. M. Jan, I. Scarfo, R. C. Larson, A. Walker, A. Schmidts, A. A. Guirguis, J. A. Gasser, M. Slabicki, A. A. Bouffard, A. P. Castano, M. C. Kann, M. L. Cabral, A. Tepper, D. E. Grinshpun, A. S. Sperling, T. Kyung, Q. L. Sievers, M. E. Birnbaum, M. V. Maus, B. L. Ebert, Reversible ON- and OFF-switch chimeric antigen receptors controlled by lenalidomide. Sci Transl Med 13, (2021).
20. J. H. Bayle, J. S. Grimley, K. Stankunas, J. E. Gestwicki, T. J. Wandless, G. R. Crabtree, Rapamycin analogs with differential binding specificity permit orthogonal control of protein activity. Chem Biol 13, 99-107 (2006).
21. P. Liu, A. Calderon, G. Konstantinidis, J. Hou, S. Voss, X. Chen, F. Li, S. Banerjee, J. E. Hoffmann, C. Theiss, L. Dehmelt, Y. W. Wu, A bioorthogonal small-molecule-switch system for controlling protein function in live cells. Angew Chem Int Ed Engl 53, 10049-10055 (2014).
22. S. Kang, K. Davidsen, L. Gomez-Castillo, H. Jiang, X. Fu, Z. Li, Y. Liang, M. Jahn, M. Moussa, F. DiMaio, L. Gu, COMBINES-CID: An Efficient Method for De Novo Engineering of Highly Specific Chemically Induced Protein Dimerization Systems. J Am Chem Soc 141, 10948-10952 (2019).
23. Z. B. Hill, A. J. Martinko, D. P. Nguyen, J. A. Wells, Human antibody-based chemically induced dimerizers for cell therapeutic applications. Nat Chem Biol 14, 112-117 (2018).
24. G. W. Foight, Z. Wang, C. T. Wei, P. Jr Greisen, K. M. Warner, D. Cunningham-Bryant, K. Park, T. J. Brunette, W. Sheffler, D. Baker, D. J. Maly, Multi-input chemical control of protein dimerization for programming graded cellular responses. Nat Biotechnol 37, 1209-1216 (2019).
25. S. Shui, P. Gainza, L. Scheller, C. Yang, Y. Kurumida, S. Rosset, S. Georgeon, R. B. Di Roberto, R. Castellanos-Rueda, S. T. Reddy, B. E. Correia, A rational blueprint for the design of chemically-controlled protein switches. Nat Commun 12, 5754 (2021).
26. M. Schapira, M. F. Calabrese, A. N. Bullock, C. M. Crews, Targeted protein degradation: expanding the toolbox. Nat Rev Drug Discov 18, 949-963 (2019).
27. X. Sun, H. Gao, Y. Yang, M. He, Y. Wu, Y. Song, Y. Tong, Y. Rao, PROTACs: great opportunities for academia and industry. Signal Transduct Target Ther 4, 64 (2019).
28. G. Weng, C. Shen, D. Cao, J. Gao, X. Dong, Q. He, B. Yang, D. Li, J. Wu, T. Hou, PROTAC-DB: an online database of PROTACs. Nucleic Acids Res 49, D1381-D1387 (2021).
29. A. Mullard, Targeted protein degraders crowd into the clinic. Nat Rev Drug Discov 20, 247-250 (2021).
30. A. Chavez, J. Scheiman, S. Vora, B. W. Pruitt, M. Tuttle, P. R. I. E, S. Lin, S. Kiani, C. D. Guzman, D. J. Wiegand, D. Ter-Ovanesyan, J. L. Braff, N. Davidsohn, B. E. Housden, N. Perrimon, R. Weiss, J. Aach, J. J. Collins, G. M. Church, Highly efficient Cas9-mediated transcriptional programming. Nat Methods 12, 326-328 (2015).
31. L. N. Gechijian, D. L. Buckley, M. A. Lawlor, J. M. Reyes, J. Paulk, C. J. Ott, G. E. Winter, M. A. Erb, T. G. Scott, M. Xu, H. S. Seo, S. Dhe-Paganon, N. P. Kwiatkowski, J. A. Perry, J. Qi, N. S. Gray, J. E. Bradner, Functional TRIM24 degrader via conjugation of ineffectual bromodomain and VHL ligands. Nat Chem Biol 14, 405-412 (2018).
32. B. Nabet, F. M. Ferguson, B. K. A. Seong, M. Kuljanin, A. L. Leggett, M. L. Mohardt, A. Robichaud, A. S. Conway, D. L. Buckley, J. D. Mancias, J. E. Bradner, K. Stegmaier, N. S. Gray, Rapid and direct control of target protein levels with VHL-recruiting dTAG molecules. Nat Commun 11, 4687 (2020).
33. M. S. Gadd, A. Testa, X. Lucas, K. H. Chan, W. Chen, D. J. Lamont, M. Zengerle, A. Ciulli, Structural basis of PROTAC cooperative recognition for selective protein degradation. Nat Chem Biol 13, 514-521 (2017).
34. M. Zengerle, K. H. Chan, A. Ciulli, Selective Small Molecule Induced Degradation of the BET Bromodomain Protein BRD4. ACS Chem Biol 10, 1770-1777 (2015).
35. C. E. Powell, Y. Gao, L. Tan, K. A. Donovan, R. P. Nowak, A. Loehr, M. Bahcall, E. S. Fischer, P. A. Janne, R. E. George, N. S. Gray, Chemically Induced Degradation of Anaplastic Lymphoma Kinase (ALK). J Med Chem 61, 4249-4255 (2018).
36. B. Nabet, J. M. Roberts, D. L. Buckley, J. Paulk, S. Dastjerdi, A. Yang, A. L. Leggett, M. A. Erb, M. A. Lawlor, A. Souza, T. G. Scott, S. Vittori, J. A. Perry, J. Qi, G. E. Winter, K. K. Wong, N. S. Gray, J. E. Bradner, The dTAG system for immediate and target-specific protein degradation. Nat Chem Biol 14, 431-441 (2018).
37. D. Remillard, D. L. Buckley, J. Paulk, G. L. Brien, M. Sonnett, H. S. Seo, S. Dastjerdi, M. Wuhr, S. Dhe-Paganon, S. A. Armstrong, J. E. Bradner, Degradation of the BAF Complex Factor BRD9 by Heterobifunctional Ligands. Angew Chem Int Ed Engl 56, 5738-5743 (2017).
38. R. P. Nowak, S. L. DeAngelo, D. Buckley, Z. He, K. A. Donovan, J. An, N. Safaee, M. P. Jedrychowski, C. M. Ponthier, M. Ishoey, T. Zhang, J. D. Mancias, N. S. Gray, J. E. Bradner, E. S. Fischer, Plasticity in binding confers selectivity in ligand-induced protein degradation. Nat Chem Biol 14, 706-714 (2018).
39. L. F. Epstein, H. Chen, R. Emkey, D. A. Whittington, The R1275Q neuroblastoma mutant and certain ATP-competitive inhibitors stabilize alternative activation loop conformations of anaplastic lymphoma kinase. J Biol Chem 287, 37447-37457 (2012).
40. J. Kronke, E. C. Fink, P. W. Hollenbach, K. J. MacBeth, S. N. Hurst, N. D. Udeshi, P. P. Chamberlain, D. R. Mani, H. W. Man, A. K. Gandhi, T. Svinkina, R. K. Schneider, M. McConkey, M. Jaras, E. Griffiths, M. Wetzler, L. Bullinger, B. E. Cathers, S. A. Carr, R. Chopra, B. L. Ebert, Lenalidomide induces ubiquitination and degradation of CK1alpha in del (5q) MDS. Nature 523, 183-188 (2015).
41. H. Gao, X. Sun, Y. Rao, PROTAC Technology: Opportunities and Challenges. ACS Med Chem Lett 11, 237-240 (2020).
42. Z. Chen, R. D. Kibler, A. Hunt, F. Busch, J. Pearl, M. Jia, Z. L. VanAernum, B. I. M. Wicky, G. Dods, H. Liao, M. S. Wilken, C. Ciarlo, S. Green, H. E1-Samad, J. Stamatoyannopoulos, V. H. Wysocki, M. C. Jewett, S. E. Boyken, D. Baker, De novo design of protein logic gates. Science 368, 78-84 (2020).
43. A. Nern, B. D. Pfeiffer, K. Svoboda, G. M. Rubin, Multiple new site-specific recombinases for use in manipulating animal genomes. Proc Natl Acad Sci USA 108, 14198-14203 (2011).
44. R. H. Friedel, W. Wurst, B. Wefers, R. Kuhn, Generating conditional knockout mice. Methods Mol Biol 693, 205-231 (2011).
45. A. V. Anzalone, L. W. Koblan, D. R. Liu, Genome editing with CRISPR-Cas nucleases, base editors, transposases and prime editors. Nat Biotechnol 38, 824-844 (2020).
46. A. V. Anzalone, P. B. Randolph, J. R. Davis, A. A. Sousa, L. W. Koblan, J. M. Levy, P. J. Chen, C. Wilson, G. A. Newby, A. Raguram, D. R. Liu, Search-and-replace genome editing without double-strand breaks or donor DNA. Nature 576, 149-157 (2019).
47. S. Agha-Mohammadi, M. O'Malley, A. Etemad, Z. Wang, X. Xiao, M. T. Lotze, Second-generation tetracycline-regulatable promoter: repositioned tet operator elements optimize transactivator synergy while shorter minimal promoter offers tight basal leakiness. J Gene Med 6, 817-828 (2004).
48. A. Costello, N. T. Lao, C. Gallagher, B. Capella Roca, L. A. N. Julius, S. Suda, J. Ducree, D. King, R. Wagner, N. Barron, M. Clynes, Leaky Expression of the TET-On System Hinders Control of Endogenous miRNA Abundance. Biotechnol J 14, e1800219 (2019).
49. S. D. Liberles, S. T. Diver, D. J. Austin, S. L. Schreiber, Inducible gene expression and protein translocation using nontoxic ligands identified by a mammalian three-hybrid screen. Proc Natl Acad Sci USA 94, 7825-7830 (1997).
50. J. R. Rubens, G. Selvaggio, T. K. Lu, Synthetic mixed-signal computation in living cells. Nat Commun 7, 11658 (2016).
51. S. Lee, N. Ding, Y. Sun, T. Yuan, J. Li, Q. Yuan, L. Liu, J. Yang, Q. Wang, A. B. Kolomeisky, I. B. Hilton, E. Zuo, X. Gao, Single C-to-T substitution using engineered APOBEC3G-nCas9 base editors with minimum genome- and transcriptome-wide off-target effects. Sci Adv 6, eaba1773 (2020).
52. M. F. Richter, K. T. Zhao, E. Eton, A. Lapinaite, G. A. Newby, B. W. Thuronyi, C. Wilson, L. W. Koblan, J. Zeng, D. E. Bauer, J. A. Doudna, D. R. Liu, Phage-assisted evolution of an adenine base editor with improved Cas domain compatibility and activity. Nat Biotechnol 38, 883-891 (2020).
53. A. M. Monteys, A. A. Hundley, P. T. Ranum, L. Tecedor, A. Muehlmatt, E. Lim, D. Lukashev, R. Sivasankaran, B. L. Davidson, Regulated control of gene therapies by drug-induced splicing. Nature 596, 291-295 (2021).
54. P. Bai, Y. Liu, S. Xue, G. C. Hamri, P. Saxena, H. Ye, M. Xie, M. Fussenegger, A fully human transgene switch to regulate therapeutic protein production by cooling sensation. Nat Med 25, 1266-1273 (2019).
55. J. Shao, S. Xue, G. Yu, Y. Yu, X. Yang, Y. Bai, S. Zhu, L. Yang, J. Yin, Y. Wang, S. Liao, S. Guo, M. Xie, M. Fussenegger, H. Ye, Smartphone-controlled optogenetically engineered cells enable semiautomatic glucose homeostasis in diabetic mice. Sci Transl Med 9, (2017).
56. H. Ye, M. Daoud-El Baba, R. W. Peng, M. Fussenegger, A synthetic optogenetic transcription device enhances blood-glucose homeostasis in mice. Science 332, 1565-1568 (2011).
57. K. N. Berrios, N. H. Evitt, R. A. DeWeerd, D. Ren, M. Luo, A. Barka, T. Wang, C. R. Bartman, Y. Lan, A. M. Green, J. Shi, R. M. Kohli, Controllable genome editing with split-engineered base editors. Nat Chem Biol 17, 1262-1270 (2021).
58. B. Zetsche, S. E. Volz, F. Zhang, A split-Cas9 architecture for inducible genome editing and transcription modulation. Nat Biotechnol 33, 139-142 (2015).
Schneider et al., NIH Image to ImageJ: 25 years of image analysis. Nat Methods 9, 671-675 (2012).
Kluesner et al., EditR: A Method to Quantify Base Editing from Sanger Sequencing. CRISPR J 1, 239-250 (2018).
Brinkman et al., Easy quantification of template-directed CRISPR/Cas9 editing. Nucleic Acids Res 46, e58 (2018).
Fischer et al., Structure of the DDB1-CRBN E3 ubiquitin ligase in complex with thalidomide. Nature 512, 49-53 (2014).

1. A system for regulating the inducible protein-protein interaction to execute a biological function, the system comprising:
(a) a first fusion protein comprising a domain of interest fused to a first interacting protein; and
(b) a second fusion protein comprising a domain of interest fused to a second interacting protein,
whereby the presence of a small molecule having a first ligand part capable of binding to the first interacting protein and a second ligand part capable of binding to the second interacting protein induces protein-protein into proximity.

2. The system of claim 1, wherein the biological function is regulating the expression of a first inducible gene, wherein the system comprises:
(a) a first fusion protein comprising a DNA binding domain of a transcription factor fused to a first interacting protein, or a nucleic acid encoding said first fusion protein;
(b) a second fusion protein comprising a transcription activator fused to a second interacting protein, or a nucleic acid encoding said second fusion protein; and
(c) a nucleic acid comprising an expression cassette wherein the first inducible gene is under the control of a promoter to which the DNA binding domain of the first fusion protein binds,
whereby the presence of a small molecule having a first ligand part capable of binding to the first interacting protein and a second ligand part capable of binding to the second interacting protein induces expression of the first inducible gene.

3. The system of claim 2, wherein the system further comprises:
(d) a third fusion protein comprising a second DNA binding domain of a transcription factor fused to a third interacting protein, or a nucleic acid encoding said third fusion protein; and
(e) a fourth fusion protein comprising a second transcription activator fused to a fourth interacting protein, or a nucleic acid encoding said fourth fusion protein;
(f) a nucleic acid comprising a second expression cassette comprising a second inducible gene is under the control of a second promoter to which the second DNA binding domain of the third fusion protein binds,
whereby the presence of a second small molecule having a third ligand capable of binding to the third interacting protein and a fourth ligand capable of binding to the fourth interacting protein induces expression of the second inducible gene.

4.-7. (canceled)

8. The system of claim 2, wherein the system further comprises:
(d) a third fusion protein comprising a second DNA binding domain of a transcription factor fused to a third interacting protein, or a nucleic acid encoding said third fusion protein; and
(e) a fourth fusion protein comprising a second transcription activator fused to a fourth interacting protein, or a nucleic acid encoding said fourth fusion protein;
wherein the first inducible gene is further under the control of a second promoter to which the second DNA binding domain of the third fusion protein binds,
whereby the presence of either (a) a first small molecule having a first ligand capable of binding to the first interacting protein and a second ligand capable of binding to the second interacting protein or (b) a second small molecule having a third ligand capable of binding to the third interacting protein and a fourth ligand capable of binding to the fourth interacting protein induces expression of the first inducible gene.

9.-14. (canceled)

15. The system of claim 2, wherein the first inducible gene is a first DNA recombinase.

16. The system of claim 15, wherein the recombinase is Cre recombinase or a Dre recombinase.

17. The system of claim 15, wherein the system further comprises a nucleic acid comprising a second expression cassette comprising a first gene of interest operably linked to a second promoter, wherein a sequence that prevents expression of the first gene of interest is positioned between the second promoter and the first gene of interest and is flanked by recombinase recognition sequences for the first DNA recombinase, wherein the first gene of interest is a second DNA recombinase, a base editor, a prime editor, or a therapeutic protein.

18.-26. (canceled)

27. The system of claim 1, wherein the biological function is inducing adenine base editing activity, wherein the system comprises:
(a) a first fusion protein comprising an N-terminal portion of an adenine base editor (ABE) deaminase domain fused to a first interacting protein, or a nucleic acid encoding said first fusion protein;
(b) a second fusion protein comprising a C-terminal portion of the ABE deaminase domain fused with a CRISPR nuclease and a second interacting protein, or a nucleic acid encoding said second fusion protein; and
wherein the presence of a small molecule having a first ligand part capable of binding to the first interacting protein and a second ligand part capable of binding to the second interacting protein induces adenine base editing activity.

28. The system of claim 27, wherein the CRISPR nuclease is SpCas9 or SpG.

29. The system of claim 27, wherein the small molecule is rapamycin.

30. The system of claim 27, wherein the first or second interaction protein is FRB or FKBP3.

31. (canceled)

32. The system of claim 1, wherein the small molecule is a proteolysis targeting chimera (PROTAC).

33. The system of claim 32, wherein one of the first interacting protein or the second interacting protein is the PROTAC's target protein, and the other of the first interacting protein or the second interacting protein is the PROTAC's E3 ubiquitin ligase.

34. The system of claim 33, wherein the E3 ubiqutin ligase (1) lacks ubiquitin ligase function; (2) lacks the seven α-helical bundle domain (HBD); or (3) is unable to interact with Damage Specific DNA Binding Protein 1 (DDB1).

35.-36. (canceled)

37. The system of claim 33, wherein the E3 ubiquitin ligase has ubiquitin ligase function.

38-39. (canceled)

40. The system of claim 33, wherein the PROTAC's target protein is the bromodomain of the target protein.

41. The system of claim 2, wherein the DNA binding domain is a GAL4 DNA binding domain, wherein the transactivation domain is a VP64-p65-Rta (VPR) transactivation domain, and/or wherein the promoter is a GAL4 cognate pUAS promoter or a tetracycline response element.

42.-43. (canceled)

44. A cell comprising the system of claim 1.

45.-54. (canceled)

55. A vector or combination of vectors comprising the nucleic acids of the system of claim 1.

56.-61. (canceled)

62. A method for producing a cell in which a first inducible gene can be inducibly expressed or in which an adenine base editor can be inducibly activated, the method comprising contacting a cell with the vector or combination of vectors of claim 55, under conditions suitable for expression of the first fusion protein and the second fusion protein.

63.-69. (canceled)


SEQUENCE LISTING (278 sequences)

SEQ ID NO: 1 - protein, 301 aa, synthetic construct
  source 1..301: note = GAL4-FKBP12^F36V
  REGION 2..8: note = SV40 NLS
  REGION 9..18: note = linker
  REGION 19..165: note = GAL4 DBD
  REGION 166..169: note = linker
  REGION 170..277: note = FKBP12F36V
  REGION 278..283: note = linker
  REGION 284..299: note = Nucleoplasmin NLS
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETSGGGSKRPAATKKAGQAKKKKSR

SEQ ID NO: 2 - protein, 1016 aa, synthetic construct
  source 1..1016: note = CRBN-VPR
  REGION 2..8: note = SV40 NLS
  REGION 9..18: note = linker
  REGION 19..459: note = CRBN
  REGION 460..463: note = linker
  REGION 464..992: note = VPR
  REGION 993..998: note = linker
  REGION 999..1014: note = Nucleoplasmin NLS
MPKKKRKVGGGSPGGGGSMAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 3 - protein, 382 aa, synthetic construct
  source 1..382: note = GAL4-VHL
  REGION 2..8: note = SV40 NLS
  REGION 9..18: note = linker
  REGION 19..165: note = GAL4 DBD
  REGION 166..169: note = linker
  REGION 170..382: note = VHL
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 4 - protein, 758 aa, synthetic construct
  source 1..758: note = TRIM24^BD-VPR
  REGION 2..8: note = SV40 NLS
  REGION 9..18: note = linker
  REGION 19..201: note = TRIM24BD
  REGION 202..205: note = linker
  REGION 206..734: note = VPR
  REGION 735..741: note = linker
  REGION 742..756: note = Nucleoplasmin NLS
MPKKKRKVGGGSPGGGGSPNEDWCAVCQNGGELLCCEKCPKVFHLSCHVPTLTNFPSGEWICTFCRDLSKPEVEYDCDAPSHNSEKKKTEGLVKLTPIDKRKCERLLLFLYCHEMSLAFQDPVPLTVPDYYKIIKNPMDLSTIKKRLQEDYSMYSKPEDFVADFRLIFQNCAEFNEPDSEVANAGIKLENYFEELLKNLYPGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 5 - protein, 1649 aa, synthetic construct
  source 1..1649: note = TRIM24-VPR
  REGION 2..8: note = SV40 NLS
  REGION 9..26: note = linker
  REGION 27..1076: note = TRIM24
  REGION 1077..1096: note = linker
  REGION 1097..1625: note = VPR
  REGION 1626..1631: note = linker
  REGION 1632..1647: note = Nucleoplasmin NLS
MPKKKRKVGGGSPGGGGSGTSRRSRTMEVAVEKAVAAAAAASAAASGGPSAAPSGENEAESRQGPDSERGGEAARLNLLDTCAVCHQNIQSRAPKLLPCLHSFCQRCLPAPQRYLMLPAPMLGSAETPPPVPAPGSPVSGSSPFATQVGVIRCPVCSQECAERHIIDNFFVKDTTEVPSSTVEKSNQVCTSCEDNAEANGFCVECVEWLCKTCIRAHQRVKFTKDHTVRQKEEVSPEAVGVTSQRPVFCPFHKKEQLKLYCETCDKLTCRDCQLLEHKEHRYQFIEEAFQNQKVIIDTLITKLMEKTKYIKFTGNQIQNRIIEVNQNQKQVEQDIKVAIFTLMVEINKKGKALLHQLESLAKDHRMKLMQQQQEVAGLSKQLEHVMHFSKWAVSSGSSTALLYSKRLITYRLRHLLRARCDASPVTNNTIQFHCDPSFWAQNIINLGSLVIEDKESQPQMPKQNPVVEQNSQPPSGLSSNQLSKFPTQISLAQLRLQHMQQQVMAQRQQVQRRPAPVGLPNPRMQGPIQQPSISHQQPPPRLINFQNHSPKPNGPVLPPHPQQLRYPPNQNIPRQAIKPNPLQMAFLAQQAIKQWQISSGQGTPSTTNSTSSTPSSPTITSAAGYDGKAFGSPMIDLSSPVGGSYNLPSLPDIDCSSTIMLDNIVRKDTNIDHGQPRPPSNRTVQSPNSSVPSPGLAGPVTMTSVHPPIRSPSASSVGSRGSSGSSSKPAGADSTHKVPVVMLEPIRIKQENSGPPENYDFPVVIVKQESDEESRPQNANYPRSILTSLLLNSSQSSTSEETVLRSDAPDSTGDQPGLHQDNSSNGKSEWLDPSQKSPLHVGETRKEDDPNEDWCAVCQNGGELLCCEKCPKVFHLSCHVPTLTNFPSGEWICTFCRDLSKPEVEYDCDAPSHNSEKKKTEGLVKLTPIDKRKCERLLLFLYCHEMSLAFQDPVPLTVPDYYKIIKNPMDLSTIKKRLQEDYSMYSKPEDFVADFRLIFQNCAEFNEPDSEVANAGIKLENYFEELLKNLYPEKRFPKPEFRNESEDNKFSDDSDDDFVQPRKKRLKSIEERQLLKLERPPDITLQGSWPASGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 6 - protein, 1297 aa, synthetic construct
  source 1..1297: note = BRD4-VPR
  REGION 2..8: note = SV40 NLS
  REGION 9..18: note = linker
  REGION 9..740: note = BRD4
  REGION 741..744: note = linker
  REGION 745..1273: note = VPR
  REGION 1274..1279: note = linker
  REGION 1280..1295: note = Nucleoplasmin NLS
MPKKKRKVGGGSPGGGGSMSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETGPAGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 7 - protein, 687 aa, synthetic construct
  source 1..687: note = BRD4^BD2-VPR
  REGION 2..8: note = SV40 NLS
  REGION 9..18: note = linker
  REGION 19..130: note = BRD4BD2
  REGION 131..134: note = linker
  REGION 135..663: note = VPR
  REGION 664..669: note = linker
  REGION 670..685: note = Nucleoplasmin NLS
MPKKKRKVGGGSPGGGGSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 8 - protein, 698 aa, synthetic construct
  source 1..698: note = BRD4^BD1-VPR
  REGION 2..8: note = SV40 NLS
  REGION 9..18: note = linker
  REGION 19..141: note = BRD4BD1
  REGION 142..145: note = linker
  REGION 146..674: note = VPR
  REGION 675..680: note = linker
  REGION 681..696: note = Nucleoplasmin NLS
MPKKKRKVGGGSPGGGGSNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 9 - protein, 301 aa, synthetic construct
  source 1..301: note = GAL4-FKBP12
  REGION 2..8: note = SV40 NLS
  REGION 9..18: note = linker
  REGION 19..165: note = GAL4 DBD
  REGION 166..169: note = linker
  REGION 170..277: note = FKBP12
  REGION 278..283: note = linker
  REGION 284..299: note = Nucleoplasmin NLS
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSGGGSKRPAATKKAGQAKKKKSR

SEQ ID NO: 10 - protein, 520 aa, synthetic construct
  source 1..520: note = GAL4-tALK
  REGION 2..8: note = SV40 NLS
  REGION 9..18: note = linker
  REGION 19..165: note = GAL4 DBD
  REGION 166..169: note = linker
  REGION 170..496: note = tALK
  REGION 497..502: note = linker
  REGION 503..518: note = Nucleoplasmin NLS
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKTSGGGSKRPAATKKAGQAKKKKSR

SEQ ID NO: 11 - protein, 788 aa, synthetic construct
  source 1..788: note = VHL-VPR
  REGION 2..8: note = SV40 NLS
  REGION 9..18: note = linker
  REGION 19..231: note = VHL
  REGION 232..235: note = linker
  REGION 236..764: note = VPR
  REGION 765..770: note = linker
  REGION 771..786: note = Nucleoplasmin NLS
MPKKKRKVGGGSPGGGGSMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 12 - protein, 790 aa, synthetic construct
  source 1..790: note = GAL4-BRD9
  REGION 2..8: note = SV40 NLS
  REGION 9..18: note = linker
  REGION 19..165: note = GAL4 DBD
  REGION 166..169: note = linker
  REGION 170..766: note = BRD9
  REGION 767..772: note = linker
  REGION 773..788: note = Nucleoplasmin NLS
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSMGKKHKKHKAEWRSSYEDYADKPLEKPLKLVLKVGGSEVTELSGSGHDSSYYDDRSDHERERHKEKKKKKKKKSEKEKHLDDEERRKRKEEKKRKREREHCDTEGEADDFDPGKKVEVEPPPDRPVRACRTQPAENESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSKQAALLGNEDTAVEEPVPEVVPVQVETAKKSKKPSREVISCMFEPEGNACSLTDSTAEEHVLALVEHAADEARDRINRFLPGGKMGYLKRNGDGSLLYSVVNTAEPDADEEETHPVDLSSLSSKLLPGFTTLGFKDERRNKVTFLSSATTALSMQNNSVFGDLKSDEMELLYSAYGDETGVQCALSLQEFVKDAGSYSKKVVDDLLDQITGGDHSRTLFQLKQRRNVPMKPPDEAKVGDTLGDSSSSVLEFMSMKSYPDVSVDISMLSSLGKVKKELDPDDSHLNLDETTKLLQDLHEAQAERGGSRPSSNLSSLSNASERDQHHLGSPSRLSVGEQPDVTHDPYEFLQSPEPAASAKTTSGGGSKRPAATKKAGQAKKKKSR

SEQ ID NO: 13 - protein, 295 aa, synthetic construct
  source 1..295: note = GAL4-BRD9^BD
  REGION 2..8: note = SV40 NLS
  REGION 9..18: note = linker
  REGION 19..165: note = GAL4 DBD
  REGION 166..169: note = linker
  REGION 170..273: note = BRD9BD
  REGION 274..277: note = linker
  REGION 278..293: note = Nucleoplasmin NLS
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSTSGGGSKRPAATKKAGQAKKKKSR

SEQ ID NO: 14 - protein, 310 aa, synthetic construct
  source 1..310: note = GAL4-FKBP3
  REGION 2..8: note = SV40 NLS
  REGION 9..18: note = linker
  REGION 19..165: note = GAL4 DBD
  REGION 166..169: note = linker
  REGION 170..286: note = FKBP3
  REGION 287..292: note = linker
  REGION 293..308: note = Nucleoplasmin NLS
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDTSGGGSKRPAATKKAGQAKKKKSR

SEQ ID NO: 15 - protein, 494 aa, synthetic construct
  source 1..494: note = GAL4-ABI
  REGION 2..8: note = SV40 NLS
  REGION 9..18: note = linker
  REGION 19..165: note = GAL4 DBD
  REGION 166..169: note = linker
  REGION 170..470: note = ABI
  REGION 471..476: note = linker
  REGION 477..492: note = Nucleoplasmin NLS
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSMVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKKLTSGGGSKRPAATKKAGQAKKKKSR

SEQ ID NO: 16 - protein, 754 aa, synthetic construct
  source 1..754: note = PYL-NPR
  REGION 2..8: note = SV40 NLS
  REGION 9..18: note = linker
  REGION 19..197: note = PYL
  REGION 198..201: note = linker
  REGION 202..730: note = VPR
  REGION 731..736: note = linker
  REGION 737..752: note = Nucleoplasmin NLS
MPKKKRKVGGGSPGGGGSAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 17 - protein, 669 aa, synthetic construct
  REGION 2..8: note = SV40 NLS
  REGION 9..18: note = linker
  REGION 19..112: note = FRB
  REGION 113..116: note = linker
  REGION 117..645: note = VPR
  REGION 646..651: note = linker
  REGION 652..667: note = Nucleoplasmin NLS
MPKKKRKVGGGSPGGGGSSSILWHEMWHEGLEEASRLYFGERNVKGMFEVLEPLHAMMERGPQTLKETSFNQAYGRDLMEAQEWCRKYMKSGNVKDLTQAWDLYYHVFRRISGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 18 - protein, 670 aa, synthetic construct
  source 1..670: note = GAL4-IKZF1
  REGION 2..8: note = SV40 NLS
  REGION 9..18: note = linker
  REGION 19..165: note = GAL4 DBD
  REGION 166..169: note = linker
  REGION 170..646: note = IKZF1
  REGION 647..652: note = linker
  REGION 653..668: note = Nucleoplasmin NLS
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGSGSMDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMSTSGGGSKRPAATKKAGQAKKKKSR

SEQ ID NO: 19 - protein, 702 aa, synthetic construct
  source 1..702: note = GAL4-IKZF3
  REGION 2..8: note = SV40 NLS
  REGION 9..18: note = linker
  REGION 19..165: note = GAL4 DBD
  REGION 166..169: note = linker
  REGION 170..678: note = IKZF3
  REGION 679..684: note = linker
  REGION 685..700: note = Nucleoplasmin NLS
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGSGSMEDIQTNAELKSTQEQSVPAESAAVLNDYSLTKSHEMENVDSGEGPANEDEDIGDDSMKVKDEYSERDENVLKSEPMGNAEEPEIPYSYSREYNEYENIKLERHVVSFDSSRPTSGKMNCDVCGLSCISFNVLMVHKRSHTGERPFQCNQCGASFTQKGNLLRHIKLHTGEKPFKCHLCNYACQRRDALTGHLRTHSVEKPYKCEFCGRSYKQRSSLEEHKERCRTFLQSTDPGDTASAEARHIKAEMGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLKTSGGGSKRPAATKKAGQAKKKKSR

SEQ ID NO: 20 - protein, 670 aa, synthetic construct
  source 1..670: note = IKZF1-GAL4
  REGION 2..8: note = SV40 NLS
  REGION 9..18: note = linker
  REGION 19..495: note = IKZF1
  REGION 496..499: note = linker
  REGION 500..646: note = GAL4 DBD
  REGION 647..652: note = linker
  REGION 653..668: note = Nucleoplasmin NLS
MPKKKRKVGGGSPGGGGSMDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMSGSGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSTSGGGSKRPAATKKAGQAKKKKSR

SEQ ID NO: 21 - protein, 702 aa, synthetic construct
  source 1..702: note = IKZF3-GAL4
  REGION 9..18: note = linker
  REGION 19..527: note = IKZF3
  REGION 528..531: note = linker
  REGION 532..678: note = GAL4 DBD
  REGION 679..684: note = linker
  REGION 685..700: note = Nucleoplasmin NLS
  REGION 2..8: note = SV40 NLS
MPKKKRKVGGGSPGGGGSMEDIQTNAELKSTQEQSVPAESAAVLNDYSLTKSHEMENVDSGEGPANEDEDIGDDSMKVKDEYSERDENVLKSEPMGNAEEPEIPYSYSREYNEYENIKLERHVVSFDSSRPTSGKMNCDVCGLSCISFNVLMVHKRSHTGERPFQCNQCGASFTQKGNLLRHIKLHTGEKPFKCHLCNYACQRRDALTGHLRTHSVEKPYKCEFCGRSYKQRSSLEEHKERCRTFLQSTDPGDTASAEARHIKAEMGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLKGSGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSTSGGGSKRPAATKKAGQAKKKKSR

SEQ ID NO: 22 - protein, 634 aa, synthetic construct
  source 1..634: note = CRBN-GAL4
  REGION 2..8: note = SV40 NLS
  REGION 9..18: note = linker
  REGION 19..459: note = CRBN
  REGION 460..463: note = linker
  REGION 464..610: note = GAL4 DBD
  REGION 611..616: note = linker
  REGION 617..632: note = Nucleoplasmin NLS
MPKKKRKVGGGSPGGGGSMAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSTSGGGSKRPAATKKAGQAKKKKSR

SEQ ID NO: 23 - protein, 634 aa, synthetic construct
  source 1..634: note = GAL4-CRBN
  REGION 2..8: note = SV40 NLS
  REGION 9..18: note = linker
  REGION 19..165: note = GAL4 DBD
  REGION 166..169: note = linker
  REGION 170..610: note = CRBN
  REGION 611..616: note = linker
  REGION 617..632: note = Nucleoplasmin NLS
MPKKKRKVGGGSPGGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSMAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLTSGGGSKRPAATKKAGQAKKKKSR

SEQ ID NO: 24 - protein, 1016 aa, synthetic construct
  source 1..1016: note = VPR-CRBN
  REGION 2..8: note = SV40 NLS
  REGION 9..18: note = linker
  REGION 19..547: note = VPR
  REGION 548..551: note = linker
  REGION 552..992: note = CRBN
  REGION 993..998: note = linker
  REGION 999..1014: note = Nucleoplasmin NLS
MPKKKRKVGGGSPGGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFGGGSMAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLTSGGGSKRPAATKKAGQAKKKKSR

SEQ ID NO: 25 - protein, 301 aa, synthetic construct
  source 1..301: note = FKBP12^F36V-GAL4
  REGION 2..8: note = SV40 NLS
  REGION 9..18: note = linker
  REGION 19..126: note = FKBP12F36V
  REGION 127..130: note = linker
  REGION 131..277: note = GAL4 DBD
  REGION 278..283: note = linker
  REGION 284..299: note = Nucleoplasmin NLS
MPKKKRKVGGGSPGGGGSMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSTSGGGSKRPAATKKAGQAKKKKSR

SEQ ID NO: 26 - protein, 683 aa, synthetic construct
  source 1..683: note = FKBP12^F36V-VPR
  REGION 2..8: note = SV40 NLS
  REGION 9..18: note = linker
  REGION 19..126: note = FKBP12F36V
  REGION 127..130: note = linker
  REGION 131..659: note = VPR
  REGION 660..665: note = linker
  REGION 666..681: note = Nucleoplasmin NLS
MPKKKRKVGGGSPGGGGSMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 27 - protein, 683 aa, synthetic construct
  source 1..683: note = VPR-FKBP12^F36V
  REGION 2..8: note = SV40 NLS
  REGION 9..18: note = linker
  REGION 19..547: note = VPR
  REGION 548..551: note = linker
  REGION 552..659: note = FKBP12F36V
  REGION 660..665: note = linker
  REGION 666..681: note = Nucleoplasmin NLS
MPKKKRKVGGGSPGGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFGGGSMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETSGGGSKRPAATKKAGQAKKKKSR

SEQ ID NO: 28 - protein, 295 aa, synthetic construct
  source 1..295: note = BRD9^BD-GAL4
  REGION 2..8: note = SV40 NLS
  REGION 9..18: note = linker
  REGION 19..120: note = BRD9BD
  REGION 121..124: note = linker
  REGION 125..271: note = GAL4 DBD
  REGION 272..277: note = linker
  REGION 278..293: note = Nucleoplasmin NLS
MPKKKRKVGGGSPGGGGSESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSGGGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSTSGGGSKRPAATKKAGQAKKKKSR

SEQ ID NO: 29 - protein, 363 aa, synthetic construct
  source 1..363: note = TetR-FKBP12^F36V
  REGION 2..8: note = SV40 NLS
  REGION 9..18: note = linker
  REGION 19..227: note = TetR
  REGION 228..231: note = linker
  REGION 232..339: note = FKBP12F36V
  REGION 340..345: note = linker
  REGION 346..361: note = Nucleoplasmin NLS
MPKKKRKVGGGSPGGGGSMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPAGGGSMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETSGGGSKRPAATKKAGQAKKKKSR

SEQ ID NO: 30 - protein, 357 aa, synthetic construct
  source 1..357: note = TetR-BRD9^BD
  REGION 2..8: note = SV40 NLS
  REGION 9..18: note = linker
  REGION 19..227: note = TetR
  REGION 228..231: note = linker
  REGION 232..333: note = BRD9BD
  REGION 334..339: note = linker
  REGION 340..355: note = Nucleoplasmin NLS
MPKKKRKVGGGSPGGGGSMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPAGGGSESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSTSGGGSKRPAATKKAGQAKKKKSR

SEQ ID NO: 31 - protein, 1593 aa, synthetic construct
  source 1..1593: note = ABE8e-SpG
  REGION 13..19: note = SV40 NLS
  REGION 20..185: note = ABE8e
  REGION 186..217: note = linker
  REGION 218..1586: note = SpG Cas9 nickase
  REGION 1587..1593: note = SV40 NLS
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGPKKKRKV

SEQ ID NO: 32 - protein, 750 aa, synthetic construct
  source 1..750: note = tCRBN-VPR-1
  REGION 2..8: note = SV40 NLS
  REGION 9..18: note = linker
  REGION 19..193: note = tCRBN
  REGION 194..197: note = linker
  REGION 198..726: note = VPR
  REGION 727..732: note = linker
  REGION 733..748: note = Nucleoplasmin NLS
MPKKKRKVGGGSPGGGGSLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 33 - protein, 958 aa, synthetic construct
  source 1..958: note = tCRBN-VPR-2
  REGION 2..8: note = SV40 NLS
  REGION 9..18: note = linker
  REGION 19..401: note = tCRBN
  REGION 402..405: note = linker
  REGION 406..934: note = VPR
  REGION 935..940: note = linker
  REGION 941..956: note = Nucleoplasmin NLS
MPKKKRKVGGGSPGGGGSMAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFTSGGGSKRPAATKKAGQAKKKKSR

SEQ ID NO: 34 - protein, 472 aa, synthetic construct
  source 1..472: note = BRD4BD2-VP64-p65
  REGION 2..8: note = SV40 NLS
  REGION 9..18: note = linker
  REGION 19..130: note = BRD4BD2
  REGION 131..134: note = linker
  REGION 135..467: note = VP64-p65
MPKKKRKVGGGSPGGGGSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDGGGSRADPKKKRKVEASGSGRADALDDFDLDMLGSDALDDFDLDMLGSDALDDFDLDMLGSDALDDFDLDMLINSRSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLTTNPG

SEQ ID NO: 35 - protein, 595 aa, synthetic construct
  source 1..595: note = Fluc with PEST Tag
  REGION 1..550: note = Fluc
  REGION 551..556: note = linker
  REGION 557..595: note = PEST Tag
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVGSTGSSHGFPPEVEEQDDGTLPMSCAQESGMDRHPAACASARINV

SEQ ID NO: 36 - protein, 239 aa, synthetic construct
  source 1..239: note = Helf1a-BFP; EBFP2
MVSKGEELFTGVVPILVELDGDVNGHKFSVRGEGEGDATNGKLTLKFICTTGKLPVPWPTLVTTLSHGVQCFARYPDHMKQHDFFKSAMPEGYVQERTIFFKDDGTYKTRAEVKFEGDTLVNRIELKGVDFKEDGNILGHKLEYNFNSHNIYIMAVKQKNGIKVNFKIRHNVEDGSVQLADHYQQNTPIGDGPVLLPDSHYLSTQSVLSKDPNEKRDHMVLLEFRTAAGITLGMDELYK

SEQ ID NO: 37 - protein, 239 aa, synthetic construct
  source 1..239: note = EYFP
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLGMDELYK

SEQ ID NO: 38 - protein, 230 aa, synthetic construct
  source 1..230: note = TRE-BFP; TagBFP
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGH

SEQ ID NO: 39 - protein, 360 aa, synthetic construct
  source 1..360: note = TRE3G-Dre
  REGION 2..8: note = SV40 NLS
  REGION 9..17: note = HA Tag
  REGION 18..360: note = Dre
MPKKKRKVYPYDVPDYAGSSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLSRSERLVDLRNRAFLFVAYNTLMRMSEISRIRVGDLDQTGDTVTLHISHTKTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMQEGTWKKPETLMRYLRRGGVSVGANSRLMDS

SEQ ID NO: 40 - protein, 356 aa, synthetic construct
  source 1..356: note = TRE3G-Cre
  REGION 2..8: note = SV40 NLS
  REGION 9..349: note = Cre
  REGION 350..356: note = SV40 NLS
MPKKKRKVNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDPKKKRKV

SEQ ID NO: 41 - protein, 302 aa, synthetic construct
  source 1..302: note = isABE-N
  REGION 2..19: note = BPSV40 NLS
  REGION 20..94: note = ABE8e-N
  REGION 95..111: note = linker
  REGION 112..203: note = FRB
  REGION 204..210: note = linker
  REGION 211..302: note = FRB
MKRTADGSEFESPKKKRKVSEVEFSHEYWMRHALTLAKRARDEREVPVGAVLVLNNRVIGEGWNRAIGLHDPTAHAEIMALRQGGLVMQNYRLIGGSGGSSGGSSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRISSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 42 - protein, 1766 aa, synthetic construct
  source 1..1766: note = isABE-C
  REGION 2..8: note = SV40 NLS
  REGION 9..125: note = FKBP3
  REGION 126..137: note = linker
  REGION 138..254: note = FKBP3
  REGION 255..266: note = linker
  REGION 267..390: note = ABE8e-C
  REGION 391..1759: note = nSpCas9
  REGION 1760..1766: note = SV40 NLS
MPKKKRKVSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGGGSSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGSSGGSSGDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGPKKKRKV

SEQ ID NO: 43 - protein, 1784 aa, synthetic construct
  source 1..1784: note = FKBP3-FKBP3-linker2-2-ABE8e-C-nSpCas9
  REGION 2..8: note = SV40 NLS
  REGION 9..125: note = FKBP3
  REGION 126..137: note = linker
  REGION 138..254: note = FKBP3
  REGION 255..285: note = linker
  REGION 286..408: note = ABE8e-C
  REGION 409..1777: note = nSpCas9
  REGION 1778..1784: note = SV40 NLS
MPKKKRKVSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGGGSSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGSSGGSSGSGGSSGSSSGGSSGGSSGDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGPKKKRKV

SEQ ID NO: 44 - protein, 1783 aa, synthetic construct
  source 1..1783: note = FKBP3-FKBP3-linker2-3-ABE8e-C-nSpCas9
  REGION 2..8: note = SV40 NLS
  REGION 9..125: note = FKBP3
  REGION 126..137: note = linker
  REGION 138..254: note = FKBP3
  REGION 255..284: note = linker
  REGION 285..407: note = ABE8e-C
  REGION 408..1776: note = nSpCas9
  REGION 1777..1783: note = SV40 NLS
MPKKKRKVSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGGGSSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGSSGGSSGPKKKRKVSGGSSGGSSGDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGPKKKRKV

SEQ ID NO: 45 - protein, 1762 aa, synthetic construct
  source 1..1762: note = FKBP3-FKBP3-linker2-4-ABE8e-C-nSpCas9
  REGION 2..8: note = SV40 NLS
  REGION 9..125: note = FKBP3
  REGION 126..137: note = linker
  REGION 138..254: note = FKBP3
  REGION 255..263: note = linker
  REGION 264..386: note = ABE8e-C
  REGION 387..1755: note = nSpCas9
  REGION 1756..1762: note = SV40 NLS
MPKKKRKVSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGGGSSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGPKKKRKV

SEQ ID NO: 46 - protein, 301 aa, synthetic construct
  source 1..301: note = ABE8eN-linker1-2-FRB-FRB
  REGION 1..19: note = BPSV40 NLS
  REGION 20..94: note = ABE8e-N
  REGION 95..110: note = linker
  REGION 111..202: note = FRB
  REGION 203..209: note = linker
  REGION 210..301: note = FRB
MKRTADGSEFESPKKKRKVSEVEFSHEYWMRHALTLAKRARDEREVPVGAVLVLNNRVIGEGWNRAIGLHDPTAHAEIMALRQGGLVMQNYRLIGSGGSSGGSSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRISSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 47 - protein, 319 aa, synthetic construct
  source 1..319: note = ABE8eN-linker1-3-FRB-FRB
  REGION 1..19: note = BPSV40 NLS
  REGION 20..94: note = ABE8e-N
  REGION 95..128: note = linker
  REGION 129..220: note = FRB
  REGION 221..227: note = linker
  REGION 228..319: note = FRB
MKRTADGSEFESPKKKRKVSEVEFSHEYWMRHALTLAKRARDEREVPVGAVLVLNNRVIGEGWNRAIGLHDPTAHAEIMALRQGGLVMQNYRLIGSGGSSGGSSGSGGSSGSSSGGSSGGSSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRISSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 48 - protein, 318 aa, synthetic construct
  source 1..318: note = ABE8eN-linker1-4-FRB-FRB
  REGION 1..19: note = BPSV40 NLS
  REGION 20..94: note = ABE8e-N
  REGION 95..127: note = linker
  REGION 128..219: note = FRB
  REGION 220..226: note = linker
  REGION 227..318: note = FRB
MKRTADGSEFESPKKKRKVSEVEFSHEYWMRHALTLAKRARDEREVPVGAVLVLNNRVIGEGWNRAIGLHDPTAHAEIMALRQGGLVMQNYRLIGSGGSSGGSSGPKKKRKVSGGSSGGSSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRISSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 49 - protein, 296 aa, synthetic construct
  source 1..296: note = ABE8eN-linker1-5-FRB-FRB
  REGION 1..19: note = BPSV40 NLS
  REGION 20..94: note = ABE8e-N
  REGION 95..105: note = linker
  REGION 106..197: note = FRB
  REGION 198..204: note = linker
  REGION 205..296: note = FRB
MKRTADGSEFESPKKKRKVSEVEFSHEYWMRHALTLAKRARDEREVPVGAVLVLNNRVIGEGWNRAIGLHDPTAHAEIMALRQGGLVMQNYRLIGGGGSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRISSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 50 - protein, 296 aa, synthetic construct; identical to SEQ ID NO: 49
  source 1..296: note = BPSV40 NLS-ABE8e-N-linker-FRB-linker-FRB
  REGION 1..19: note = BPSV40 NLS
  REGION 20..94: note = ABE8e-N
  REGION 95..105: note = linker
  REGION 106..197: note = FRB
  REGION 198..204: note = linker
  REGION 205..296: note = FRB

SEQ ID NO: 51 - protein, 146 aa, synthetic construct
  source 1..146: note = ABE8eN (1-25)-linker-FRB
  REGION 1..19: note = BPSV40 NLS
  REGION 20..43: note = ABE8e-N
  REGION 44..54: note = linker
  REGION 55..146: note = FRB
MKRTADGSEFESPKKKRKVSEVEFSHEYWMRHALTLAKRARDEGGGGSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 52 - protein, 194 aa, synthetic construct
  source 1..194: note = ABE8E (1-73)-linker-FRB
  REGION 1..19: note = BPSV40 NLS
  REGION 20..91: note = ABE8e-N
  REGION 92..102: note = linker
  REGION 103..194: note = FRB
MKRTADGSEFESPKKKRKVSEVEFSHEYWMRHALTLAKRARDEREVPVGAVLVLNNRVIGEGWNRAIGLHDPTAHAEIMALRQGGLVMQNYGGGGSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 53 - protein, 195 aa, synthetic construct
  source 1..195: note = ABE8E (1-74)-linker-FRB
  REGION 1..19: note = BPSV40 NLS
  REGION 20..92: note = ABE8e-N
  REGION 93..103: note = linker
  REGION 104..195: note = FRB
MKRTADGSEFESPKKKRKVSEVEFSHEYWMRHALTLAKRARDEREVPVGAVLVLNNRVIGEGWNRAIGLHDPTAHAEIMALRQGGLVMQNYRGGGGSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 54 - protein, 196 aa, synthetic construct
  source 1..196: note = ABE8E (1-75)-linker-FRB
  REGION 1..19: note = BPSV40 NLS
  REGION 20..93: note = ABE8e-N
  REGION 94..104: note = linker
  REGION 105..196: note = FRB
MKRTADGSEFESPKKKRKVSEVEFSHEYWMRHALTLAKRARDEREVPVGAVLVLNNRVIGEGWNRAIGLHDPTAHAEIMALRQGGLVMQNYRLGGGGSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 55 - protein, 197 aa, synthetic construct
  source 1..197: note = ABE8E (1-76)-linker-FRB
  REGION 1..19: note = BPSV40 NLS
  REGION 20..94: note = ABE8e-N
  REGION 95..105: note = linker
  REGION 106..197: note = FRB
MKRTADGSEFESPKKKRKVSEVEFSHEYWMRHALTLAKRARDEREVPVGAVLVLNNRVIGEGWNRAIGLHDPTAHAEIMALRQGGLVMQNYRLIGGGGSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 56 - protein, 198 aa, synthetic construct
  source 1..198: note = ABE8E (1-77)-linker-FRB
  REGION 1..19: note = BPSV40 NLS
  REGION 20..95: note = ABE8e-N
  REGION 96..106: note = linker
  REGION 107..198: note = FRB
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDGGGGSGGGGSSILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 57 - protein, 1684 aa, synthetic construct
  source 1..1684: note = FKBP3-linker-ABE8e-26-C-nSpCas9
  REGION 2..8: note = SV40 NLS
  REGION 9..125: note = FKBP3
  REGION 126..134: note = linker
  REGION 135..308: note = ABE8e-C
  REGION 309..1677: note = nSpCas9
  REGION 1678..1684: note = SV40 NLS
MPKKKRKVSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGPKKKRKV

SEQ ID NO: 58 - protein, 1765 aa, synthetic construct
  source 1..1765: note = FKBP3-linker-ABE8e-73-C-nSpCas9
  REGION 2..8: note = SV40 NLS
  REGION 9..125: note = FKBP3
  REGION 126..137: note = linker
  REGION 138..254: note = FKBP3
  REGION 255..263: note = linker
  REGION 264..389: note = ABE8e-C
  REGION 390..1758: note = nSpCas9
  REGION 1759..1765: note = SV40 NLS
MPKKKRKVSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGGGSSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGPKKKRKV

SEQ ID NO: 59 - protein, 1764 aa, synthetic construct
  source 1..1764: note = FKBP3-linker-ABE8e-74-C-nSpCas9
  REGION 2..8: note = SV40 NLS
  REGION 9..125: note = FKBP3
  REGION 126..137: note = linker
  REGION 138..254: note = FKBP3
  REGION 255..263: note = linker
  REGION 264..388: note = ABE8e-C
  REGION 389..1757: note = nSpCas9
  REGION 1758..1764: note = SV40 NLS
MPKKKRKVSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGGGSSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGPKKKRKV

SEQ ID NO: 60 - protein, 1763 aa, synthetic construct
  source 1..1763: note = FKBP3-linker-ABE8e-75-C-nSpCas9
  REGION 2..8: note = SV40 NLS
  REGION 9..125: note = FKBP3
  REGION 126..137: note = linker
  REGION 138..254: note = FKBP3
  REGION 255..263: note = linker
  REGION 264..387: note = ABE8e-C
  REGION 388..1756: note = nSpCas9
  REGION 1757..1763: note = SV40 NLS
MPKKKRKVSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGGGSSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGPKKKRKV

SEQ ID NO: 61 - protein, 1762 aa, synthetic construct; identical to SEQ ID NO: 45
  source 1..1762: note = FKBP3-linker-ABE8e-76-C-nSpCas9
  REGION 2..8: note = SV40 NLS
  REGION 9..125: note = FKBP3
  REGION 126..137: note = linker
  REGION 138..254: note = FKBP3
  REGION 255..263: note = linker
  REGION 264..386: note = ABE8e-C
  REGION 387..1755: note = nSpCas9
  REGION 1756..1762: note = SV40 NLS

SEQ ID NO: 62 - protein, 1761 aa, synthetic construct
  source 1..1761: note = FKBP3-linker-ABE8e-77-C-nSpCas9
  REGION 2..8: note = SV40 NLS
  REGION 9..125: note = FKBP3
  REGION 126..137: note = linker
  REGION 138..254: note = FKBP3
  REGION 255..263: note = linker
  REGION 264..385: note = ABE8e-C
  REGION 386..1754: note = nSpCas9
  REGION 1755..1761: note = SV40 NLS
MPKKKRKVSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGGGSSPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDGGGSGGGSGATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGPKKKRKV

SEQ ID NO: 63 - DNA, 47 bp, synthetic construct
  source 1..47: mol_type = other DNA; note = GAL4-F-d270
tagcatggtctcactcaatgaagctactgtcttctatcgaacaagca

SEQ ID NO: 64 - DNA, 43 bp, synthetic construct
  source 1..43: mol_type = other DNA; note = GAL4-R-d270
ataggtctctcgatacagtcaactgtctttgacctttgttact

SEQ ID NO: 65 - DNA, 53 bp, synthetic construct
  source 1..53: mol_type = other DNA; note = VHL-F-d270
tagtaggtctcaatcgggtggcggctcaatgccccggagggcggagaactggg

SEQ ID NO: 66 - DNA, 37 bp, synthetic construct
  source 1..37: mol_type = other DNA; note = VHL-R-d270
tagtaggtctcatagtggcaatcaatctcccatccgt

SEQ ID NO: 67 - DNA, 57 bp, synthetic construct
  source 1..57: mol_type = other DNA; note = TRIM24-1-F
tagtggtctcactcaggtacctctagaagatctaggacaatggaggtggcggtggag

SEQ ID NO: 68 - DNA, 48 bp, synthetic construct
  source 1..48: mol_type = other DNA; note = TRIM24-1-R
tagtaggtctcacggagtctcggccgagcccagcatgggcgcgggcag

SEQ ID NO: 69 - DNA, 29 bp, synthetic construct
  source 1..29: mol_type = other DNA; note = TRIM24-2-F
tagtaggtctcatccgccacccgtccctg

SEQ ID NO: 70 - DNA, 46 bp, synthetic construct
  source 1..46: mol_type = other DNA; note = TRIM24-m-R
agtaggtctcacattctagggtttggtaaacccacaggtgctggcc

SEQ ID NO: 71 - DNA, 33 bp, synthetic construct
  source 1..33: mol_type = other DNA; note = TRIM24-m-F
tagtaggtctcaaatgcaggggcccatccagca

SEQ ID NO: 72 - DNA, 57 bp, synthetic construct
  source 1..57: mol_type = other DNA; note = TRIM24-2-R-c
taggtctcacaccgctagctggccaggatccctgcagggtgatatcgggcggccgct

SEQ ID NO: 73 - DNA, 47 bp, synthetic construct
  source 1..47: mol_type = other DNA; note = VPR-F
tagtaggtctcaggtggcgggtcgagagctgaccccaagaagaagag

SEQ ID NO: 74 - DNA, 59 bp, synthetic construct
  source 1..59: mol_type = other DNA; note = VPR(L)-R
tagggtctcatagtaaacaaacttgtgtcaaatatactcagcccggtgctgatgtgcat

SEQ ID NO: 75 - DNA, 59 bp, synthetic construct
  source 1..59: mol_type = other DNA; note = BRD4-Front-F
aggtctcactcaatgtctgcggagagcggccctgggacgagattgagaaatctgccagt

SEQ ID NO: 76 - DNA, 59 bp, synthetic construct
  source 1..59: mol_type = other DNA; note = BRD4-front-R
tagtggtctcacaccagcgggacctgtttcggagtcttcgctgtcagaggagctggact

SEQ ID NO: 77 - DNA, 47 bp, synthetic construct; identical to SEQ ID NO: 73
  source 1..47: mol_type = other DNA; note = VPR-behind-F

SEQ ID NO: 78 - DNA, 59 bp, synthetic construct; identical to SEQ ID NO: 74
  source 1..59: mol_type = other DNA; note = VPR-behind-R

SEQ ID NO: 79 - DNA, 47 bp, synthetic construct; identical to SEQ ID NO: 63
  source 1..47: mol_type = other DNA; note = GAL4-F-d270

SEQ ID NO: 80 - DNA, 43 bp, synthetic construct; identical to SEQ ID NO: 64
  source 1..43: mol_type = other DNA; note = GAL4-R-d270

SEQ ID NO: 81 - DNA, 51 bp, synthetic construct
  source 1..51: mol_type = other DNA; note = ALK-1-F-d351-c
tatagtggtctcaatcgggtggcggctcacgcacctcgaccatcatgaccg

SEQ ID NO: 82 - DNA, 49 bp, synthetic construct
  source 1..49: mol_type = other DNA; note = ALK-1-R-d351
tataggtctcagaggaaggacttgagatctccccccgccatgagctcca

SEQ ID NO: 83 - DNA, 53 bp, synthetic construct
  source 1..53: mol_type = other DNA; note = ALK-2-F-d351
tagtacggtctcacctccgagaaacccgccctcgcccgagccagccctcctcc

SEQ ID NO: 84 - DNA, 56 bp, synthetic construct
  source 1..56: mol_type = other DNA; note = ALK-2-R-d351
tagtaggtctcatagttttctcttcctcttccacaagtggaccatattctatcggc

SEQ ID NO: 85 - DNA, 41 bp, synthetic construct
  source 1..41: mol_type = other DNA; note = VHL-front-F
tagtaggtctcactcaatgccccggagggcggagaactggg

SEQ ID NO: 86 - DNA, 48 bp, synthetic construct
  source 1..48: mol_type = other DNA; note = VHL-front-R
tagtaggtctcacaccatctcccatccgttgatgtgcaatgcgctcct

SEQ ID NO: 87 - DNA, 47 bp, synthetic construct; identical to SEQ ID NO: 73
  source 1..47: mol_type = other DNA; note = VPR-behind-F

SEQ ID NO: 88 - DNA, 59 bp, synthetic construct; identical to SEQ ID NO: 74
  source 1..59: mol_type = other DNA; note = VPR-behind-R

SEQ ID NO: 89 - DNA, 47 bp, synthetic construct; identical to SEQ ID NO: 63
  source 1..47: mol_type = other DNA; note = GAL4-F

SEQ ID NO: 90 - DNA, 43 bp, synthetic construct; identical to SEQ ID NO: 64
  source 1..43: mol_type = other DNA; note = GAL4-R

SEQ ID NO: 91 - DNA, 53 bp, synthetic construct
  source 1..53: mol_type = other DNA; note = BRD9-1-F
tagtaggtctcaatcgggtggcggctcaatgggcaagaagcacaagaagcaca

SEQ ID NO: 92 - DNA, 56 bp, synthetic construct
  source 1..56: mol_type = other DNA; note = BRD9-R
tagtaggtctcatagtggtcttggcagaggccgcaggctctggagactgaagaaac

SEQ ID NO: 93 - DNA, 59 bp, synthetic construct
  source 1..59: mol_type = other DNA; note = BRD4-behind-F-c
tagtaggtctcaggtggcggctcaatgtctgcggagagcggccctgggacgagattgag

SEQ ID NO: 94 - DNA, 59 bp, synthetic construct
  source 1..59: mol_type = other DNA; note = BRD4-behind-R-c
agatggtctcatagtagcgggacctgtttcggagtcttcgctgtcagaggagctggact

SEQ ID NO: 95 - DNA, 45 bp, synthetic construct
  source 1..45: mol_type = other DNA; note = GAL4-front-F
agtaggtctcactcaatgaagctactgtcttctatcgaacaagca

SEQ ID NO: 96 - DNA, 49 bp, synthetic construct
  source 1..49: mol_type = other DNA; note = GAL4-front-R
tagtaggtctcacacccgatacagtcaactgtctttgacctttgttact

SEQ ID NO: 97 - DNA, 48 bp, synthetic construct
  source 1..48: mol_type = other DNA; note = ABI-F
tagtaggtctcaggtggcggctcaatggtgcctttgtatggttttact

SEQ ID NO: 98 - DNA, 41 bp, synthetic construct
  source 1..41: mol_type = other DNA; note = ABI-R
tagtaggtctcatagttagcttcttcaaatcaaccaccacc

SEQ ID NO: 99 - DNA, 45 bp, synthetic construct; identical to SEQ ID NO: 95
  source 1..45: mol_type = other DNA; note = GAL4-front-F

SEQ ID NO: 100 - DNA, 49 bp, synthetic construct; identical to SEQ ID NO: 96
  source 1..49: mol_type = other DNA; note = GAL4-front-R

SEQ ID NO: 101 - DNA, 33 bp, synthetic construct
  source 1..33: mol_type = other DNA; note = PYL-F
tagtaggtctcactcagcgccaactcaagacga

SEQ ID NO: 102 - DNA, 59 bp, synthetic construct; identical to SEQ ID NO: 74
  source 1..59: mol_type = other DNA; note = VPR(L)-R

SEQ ID NO: 103 - DNA, 51 bp, synthetic construct
  source 1..51: mol_type = other DNA; note = FKBP3-F
tatagtaggtctcaatcgggtggcggctcaagccccaagtacaccaagagc

SEQ ID NO: 104 - DNA, 41 bp, synthetic construct
  source 1..41: mol_type = other DNA; note = FKBP3-R
tagtaggtctcatagtgtcgatgtccaccagctccacctca

SEQ ID NO: 105 - DNA, 47 bp, synthetic construct; identical to SEQ ID NO: 63
  source 1..47: mol_type = other DNA; note = GAL4-F

SEQ ID NO: 106 - DNA, 43 bp, synthetic construct; identical to SEQ ID NO: 64
  source 1..43: mol_type = other DNA; note = GAL4-R

SEQ ID NO: 107 - DNA, 45 bp, synthetic construct; identical to SEQ ID NO: 95
  source 1..45: mol_type = other DNA; note = GAL4-front-F

SEQ ID NO: 108 - DNA, 49 bp, synthetic construct; identical to SEQ ID NO: 96
  source 1..49: mol_type = other DNA; note = GAL4-front-R

SEQ ID NO: 109 - DNA, 45 bp, synthetic construct
  source 1..45: mol_type = other DNA; note = FKBP12F36V-behind-F
tagtaggtctcaggtggcggctcaatgggagtgcaggtggaaacc

SEQ ID NO: 110 - DNA, 47 bp, synthetic construct
  source 1..47: mol_type = other DNA
tagtaggtctcatagtttccagttttagaagctccacatcgaagacg

SEQ ID NO: 111 - DNA, 34 bp, synthetic construct
  source 1..34: mol_type = other DNA; note = FRB-F
taatgtggtctcactcatcgagcatcctgtggca

SEQ ID NO: 112 - DNA, 39 bp, synthetic construct
  source 1..39: mol_type = other DNA; note = FRB-R
tagtaggtctcacaccgctgatcctcctgaacacgtggt

SEQ ID NO: 113 - DNA, 47 bp, synthetic construct; identical to SEQ ID NO: 73
  source 1..47: mol_type = other DNA; note = VPR-behind-F

SEQ ID NO: 114 - DNA, 59 bp, synthetic construct; identical to SEQ ID NO: 74
  source 1..59: mol_type = other DNA; note = VPR-behind-R

SEQ ID NO: 115 - DNA, 36 bp, synthetic construct
  source 1..36: mol_type = other DNA; note = TRIM-F
tagtaggtctcactcacccaatgaggactggtgtgc

SEQ ID NO: 116 - DNA, 44 bp, synthetic construct
  source 1..44: mol_type = other DNA; note = TRIM-R
tagtaggtctcacacctggatagaggttctttagaagttcttca

SEQ ID NO: 117 - DNA, 59 bp, synthetic construct; identical to SEQ ID NO: 74
  source 1..59: mol_type = other DNA; note = VPR(L)-R

SEQ ID NO: 118 - DNA, 47 bp, synthetic construct; identical to SEQ ID NO: 73
  source 1..47: mol_type = other DNA; note = VPR-F

SEQ ID NO: 119 - DNA, 38 bp, synthetic construct
  source 1..38: mol_type = other DNA; note = BRD4-BD1-F
tagtaggtctcactcaaaccccccgcccccagagactt

SEQ ID NO: 120 - DNA, 59 bp, synthetic construct; identical to SEQ ID NO: 74
  source 1..59: mol_type = other DNA; note = VPR-R

SEQ ID NO: 121 - DNA, 34 bp, synthetic construct
  source 1..34: mol_type = other DNA; note = BRD4-BD2-F
tataggtctcactcaagcaaggtgtcggagcagc

SEQ ID NO: 122 - DNA, 59 bp, synthetic construct; identical to SEQ ID NO: 74
  source 1..59: mol_type = other DNA; note = VPR-R

SEQ ID NO: 123 - DNA, 47 bp, synthetic construct; identical to SEQ ID NO: 63
  source 1..47: mol_type = other DNA; note = GAL4-F

SEQ ID NO: 124 - DNA, 43 bp, synthetic construct; identical to SEQ ID NO: 64
  source 1..43: mol_type = other DNA; note = GAL4-R

SEQ ID NO: 125 - DNA, 54 bp, synthetic construct
  source 1..54: mol_type = other DNA; note = tBRD9-F
tagtaggtctcaatcgggtggcggctcagagagcacacctattcagcaactcct

SEQ ID NO: 126 - DNA, 50 bp, synthetic construct
  source 1..50: mol_type = other DNA; note = tBRD9-R
tagtggtctcatagtgctcatcatcttaaagcctgcgtgaaggatcttct

SEQ ID NO: 127 - DNA, 36 bp, synthetic construct
  source 1..36: mol_type = other DNA; note = UAS-F
agcgaggtctcactattgcaggcgataggtaccgag

SEQ ID NO: 128 - DNA, 34 bp, synthetic construct
  source 1..34: mol_type = other DNA; note = UAS-R
agcgaggtctcaaagcgcgcccagtaccggaatg

SEQ ID NO: 129 - DNA, 32 bp, synthetic construct
  source 1..32: mol_type = other DNA; note = Fluc-F
tagtaggtctcagcttggcaatccggtactgt

SEQ ID NO: 130 - DNA, 41 bp, synthetic construct
  source 1..41: mol_type = other DNA; note = Luci-R
tataggtctcaatcccacggcgatcttgccgcccttcttgg

SEQ ID NO: 131 - DNA, 56 bp, synthetic construct
  source 1..56: mol_type = other DNA; note = PEST-R
atagggtctcatagttacacattgatcctagcagaagcacaggctgcagggtgacg

SEQ ID NO: 132 - DNA, 59 bp, synthetic construct
  source 1..59: mol_type = other DNA
tagggtctcaggatccaccggatctagccatggcttcccgccggaggtggaggagcagg

SEQ ID NO: 133 - DNA, 51 bp, synthetic construct
  source 1..51: mol_type = other DNA; note = EYFP-F
tagtaggtctcagcttatggtgagcaagggcgaggagctgttcaccggggt

SEQ ID NO: 134 - DNA, 42 bp, synthetic construct
  source 1..42: mol_type = other DNA; note = EYFP-R
atacgtaggtctcatagtttacttgtacagctcgtccatgcc

SEQ ID NO: 135 - DNA, 40 bp, synthetic construct
  source 1..40: mol_type = other DNA; note = TRE-F
agcgaggtctcactcattgtatagaaaagttggctccgaa

SEQ ID NO: 136 - DNA, 35 bp, synthetic construct
  source 1..35: mol_type = other DNA
agcgaggtctcaaagccgaccggtaatcagcctgc

SEQ ID NO: 137 - DNA, 34 bp, synthetic construct
  source 1..34: mol_type = other DNA; note = UAS-F
tagtaggtctcactcagcagtttatggcgggcgt

SEQ ID NO: 138 - DNA, 34 bp, synthetic construct
  source 1..34: mol_type = other DNA; note = 569-6-1-R
tagtaggtctcaggtcccgacatggtggctcagc

SEQ ID NO: 139 - DNA, 58 bp, synthetic construct
  source 1..58: mol_type = other DNA; note = dre-R
tagtaggtctcatagtgccacaatcagctatccatcagtcgagaattggctcccacgg

SEQ ID NO: 140 - DNA, 59 bp, synthetic construct
  source 1..59: mol_type = other DNA
tagtggtctcagaccatgcctaagaagaagaggaaggtttacccatacgatgttccaga

SEQ ID NO: 141 - DNA, 42 bp, synthetic construct
  source 1..42: mol_type = other DNA; note = TRE-3G-R
tagtaggtctcaggtcactttacgagggtaggaagtggtacg

SEQ ID NO: 142 - DNA, 39 bp, synthetic construct
  source 1..39: mol_type = other DNA; note = TRE3G-F
tagtaggtctcactcattcgtcttcaagaattcctcgag

SEQ ID NO: 143 - DNA, 59 bp, synthetic construct
  source 1..59: mol_type = other DNA; note = TRE-cre-F
aggtctcagaccatgccaaagaagaagcggaaggtcaatttactgaccgtacaccaaaa

SEQ ID NO: 144 - DNA, 59 bp, synthetic construct
  source 1..59: mol_type = other DNA; note = CreNLS-R-ACTA
taggtctcatagtttagaccttccgcttcttcttatcgccatcttccagcaggcgcacc

SEQ ID NO: 145 - DNA, 39 bp, synthetic construct; identical to SEQ ID NO: 142
  source 1..39: mol_type = other DNA; note = TRE-F

SEQ ID NO: 146 - DNA, 58 bp, synthetic construct; identical to SEQ ID NO: 139
  source 1..58: mol_type = other DNA

SEQ ID NO: 147 - DNA, 59 bp, synthetic construct; identical to SEQ ID NO: 144
  source 1..59: mol_type = other DNA; note = CreNLS-R-ACTA

SEQ ID NO: 148 - DNA, 39 bp, synthetic construct; identical to SEQ ID NO: 142
  source 1..39: mol_type = other DNA; note = TRE-F

SEQ ID NO: 149 - DNA, 39 bp, synthetic construct; identical to SEQ ID NO: 142
  source 1..39: mol_type = other DNA

SEQ ID NO: 150 - DNA, 42 bp, synthetic construct; identical to SEQ ID NO: 141
  source 1..42: mol_type = other DNA; note = TRE-3G-R

SEQ ID NO: 151 - DNA, 33 bp, synthetic construct
  source 1..33: mol_type = other DNA; note = A3G-1-F
tagtaggtctcagacctagagatccgcggccgc

SEQ ID NO: 152 - DNA, 31 bp, synthetic construct
  source 1..31: mol_type = other DNA; note = A3G-m-R
tagtaggtctcagtttcgtgtctgccccgca

SEQ ID NO: 153 - DNA, 51 bp, synthetic construct
  source 1..51: mol_type = other DNA; note = A3G-m-F
tagtaggtctcaaaacctacctgtgctatgaggtggagcggatgcacaacg

SEQ ID NO: 154 - DNA, 45 bp, synthetic construct
  source 1..45: mol_type = other DNA; note = A3G-2-R
tagtaggtctcacctttttctgctcgccgctcaggaaggcgggct

SEQ ID NO: 155 - DNA, 34 bp, synthetic construct
  source 1..34: mol_type = other DNA; note = A3G-3-F
tagtaggtctcaaaggccatcgtggacctgctgt

SEQ ID NO: 156 - DNA, 34 bp, synthetic construct
  source 1..34: mol_type = other DNA; note = A3G-3-R
tagtaggtctcatagtaggcacagtcgaggctga

SEQ ID NO: 157 - DNA, 39 bp, synthetic construct; identical to SEQ ID NO: 142
  source 1..39: mol_type = other DNA; note = TRE3G-F

SEQ ID NO: 158 - DNA, 42 bp, synthetic construct; identical to SEQ ID NO: 141
  source 1..42: mol_type = other DNA; note = TRE-3G-R-GACC

SEQ ID NO: 159 - DNA, 34 bp, synthetic construct
  source 1..34: mol_type = other DNA; note = ABE(TRE-F)-gacc
tatgtggtctcagaccggagagccgccaccatga

SEQ ID NO: 160 - DNA, 41 bp, synthetic construct
  source 1..41: mol_type = other DNA; note = SpG-1-R
tatgaggtctcaccacgtacatatcccgcccattctgcagg

SEQ ID NO: 161 - DNA, 39 bp, synthetic construct
  source 1..39: mol_type = other DNA; note = SpG-2-F
tagtggtctcagtggaccaggaactggacatcaaccggc

SEQ ID NO: 162 - DNA, 56 bp, synthetic construct
  source 1..56: mol_type = other DNA
tagtggtctcatagtttagactttcctcttcttcttggggccgctgtcacctccca

SEQ ID NO: 163 - DNA, 39 bp, synthetic construct; identical to SEQ ID NO: 142
  source 1..39: mol_type = other DNA

SEQ ID NO: 164 - DNA, 42 bp, synthetic construct; identical to SEQ ID NO: 141
  source 1..42: mol_type = other DNA

SEQ ID NO: 165 - DNA, 34 bp, synthetic construct
  source 1..34: mol_type = other DNA
taagtaggtctcagaccggtcgacccgccaccaa

SEQ ID NO: 166 - DNA, 29 bp, synthetic construct
  source 1..29: mol_type = other DNA
tagtaggtctcatgggctgcaggtcgagg

SEQ ID NO: 167 - DNA, 34 bp, synthetic construct
  source 1..34: mol_type = other DNA
tagtaggtctcacccaagccgccaccatgaaacg

SEQ ID NO: 168 - DNA, 41 bp, synthetic construct; identical to SEQ ID NO: 160
  source 1..41: mol_type = other DNA

SEQ ID NO: 169 - DNA, 39 bp, synthetic construct; identical to SEQ ID NO: 161
  source 1..39: mol_type = other DNA

SEQ ID NO: 170 - DNA, 56 bp, synthetic construct; identical to SEQ ID NO: 162
  source 1..56: mol_type = other DNA

SEQ ID NO: 171 - DNA, 42 bp, synthetic construct; identical to SEQ ID NO: 141
  source 1..42: mol_type = other DNA

SEQ ID NO: 172 - DNA, 39 bp, synthetic construct; identical to SEQ ID NO: 142
  source 1..39: mol_type = other DNA

SEQ ID NO: 173 - DNA, 45 bp, synthetic construct
  source 1..45: mol_type = other DNA
tagtaggtctcagaccatgaaacggacagccgacggaagcgagtt

SEQ ID NO: 174 - DNA, 49 bp, synthetic construct
  source 1..49: mol_type = other DNA
atgatggtctcatcatgatgttgctgtagaagaagtacttggcggtagc

SEQ ID NO: 175 - DNA, 44 bp, synthetic construct
  source 1..44: mol_type = other DNA
agtaggtctcaatgaactttttcaagaccgagattaccctggcc

SEQ ID NO: 176 - DNA, 33 bp, synthetic construct
  source 1..33: mol_type = other DNA
tagtaggtctcagtctcatgtagccgatactca

SEQ ID NO: 177 - DNA, 55 bp, synthetic construct
  source 1..55: mol_type = other DNA
tagtaggtctcaagacttcaaaagagccagatgtttctctagggtccacatggct

SEQ ID NO: 178 - DNA, 35 bp, synthetic construct
  source 1..35: mol_type = other DNA
tagtaggtctcatagtaaggcacagtcgaggctga

SEQ ID NO: 179 - DNA, 51 bp, synthetic construct
  source 1..51: mol_type = other DNA
atagtggtctcactcaatgaatttactgaccgtacaccaaaatttgcctgc

SEQ ID NO: 180 - DNA, 45 bp, synthetic construct
  source 1..45: mol_type = other DNA
accaggttcgttcactcatggaaaatagcgatcgctgccaggata

SEQ ID NO: 181 - DNA, 44 bp, synthetic construct
  source 1..44: mol_type = other DNA
tagtaggtctcagatacgtaatctggcatttctggggattgctt

SEQ ID NO: 182 - DNA, 59 bp, synthetic construct
  source 1..59: mol_type = other DNA
actaggtctcttagtttagactttccgcttcttctttggatcgccatcttccagcaggc

SEQ ID NO: 183 - DNA, 37 bp, synthetic construct
  source 1..37: mol_type = other DNA
agtggtctcactcatgatagtaggaggcttggtaggt

SEQ ID NO: 184 - DNA, 31 bp, synthetic construct
  source 1..31: mol_type = other DNA
tagtaggtctcccggtgtttcgtcctttcca

SEQ ID NO: 185 - DNA, 37 bp, synthetic construct; identical to SEQ ID NO: 183
  source 1..37: mol_type = other DNA

SEQ ID NO: 186 - DNA, 31 bp, synthetic construct; identical to SEQ ID NO: 184
  source 1..31: mol_type = other DNA

SEQ ID NO: 187 - DNA, 48 bp, synthetic construct
  source 1..48: mol_type = other DNA
ctcaggtctcactatatttggctccggtgcccgtcagtgggcagagcg

SEQ ID NO: 188 - DNA, 31 bp, synthetic construct
  source 1..31: mol_type = other DNA
tagtaggtctcatacctgtgttctggcggca

SEQ ID NO: 189 - DNA, 34 bp, synthetic construct
  source 1..34: mol_type = other DNA
agtgggtctcaacgggaaagctgggtagatcagt

SEQ ID NO: 190 - DNA, 33 bp, synthetic construct
  source 1..33: mol_type = other DNA
tagtaggtctcaggtacaccggtgccaccatgc

SEQ ID NO: 191 - DNA, 37 bp, synthetic construct
  source 1..37: mol_type = other DNA
tagtaggtctcagcagaagtgggttctctagttagcc

SEQ ID NO: 192 - DNA, 38 bp, synthetic construct
  source 1..38: mol_type = other DNA
ctcaggtctcaccgtacgtatgttcccatagtaacgcc

SEQ ID NO: 193 - DNA, 48 bp, synthetic construct
  source 1..48: mol_type = other DNA
tagtggtctcatagtcaacagggcgctaaaatccatgtcggcgattga

SEQ ID NO: 194 - DNA, 32 bp, synthetic construct
  source 1..32: mol_type = other DNA
tataggtctcactgccaccggtgccaccatgc

SEQ ID NO: 195 - DNA, 48 bp, synthetic construct; identical to SEQ ID NO: 193
  source 1..48: mol_type = other DNA

SEQ ID NO: 196 - DNA, 32 bp, synthetic construct; identical to SEQ ID NO: 194
  source 1..32: mol_type = other DNA

SEQ ID NO: 197 - DNA, 36 bp, synthetic construct
  source 1..36: mol_type = other DNA
tagtaggtctcagactggcgtgtacggtgggcgcct

SEQ ID NO: 198 - DNA, 42 bp, synthetic construct; identical to SEQ ID NO: 134
  source 1..42: mol_type = other DNA

SEQ ID NO: 199 - DNA, 47 bp, synthetic construct; identical to SEQ ID NO: 63
  source 1..47: mol_type = other DNA

SEQ ID NO: 200 - DNA, 49 bp, synthetic construct
  source 1..49: mol_type = other DNA
tagtaggtctcaagcccgatacagtcaactgtctttgacctttgttact

SEQ ID NO: 201 - DNA, 47 bp, synthetic construct
  source 1..47: mol_type = other DNA
tagtaggtctcaggctcaggctcaatggatgctgatgagggtcaaga

SEQ ID NO: 202 - DNA, 41 bp, synthetic construct
  source 1..41: mol_type = other DNA
tagtaggtctcatagtgctcatgtggaagcggtgctcccct

SEQ ID NO: 203 - DNA, 59 bp, synthetic construct
  source 1..59: mol_type = other DNA
ataggtctcaggctcaggctcaatggaagatatacaaacaaatgcggaactgaaaagca

SEQ ID NO: 204 - DNA, 41 bp, synthetic construct
  source 1..41: mol_type = other DNA
tagtaggtctcatagtcttcagcagggctctgtgttctcct

SEQ ID NO: 205 - DNA, 43 bp, synthetic construct
  source 1..43: mol_type = other DNA
tagcatggtctcaatgaagctactgtcttctatcgaacaagca

SEQ ID NO: 206 - DNA, 49 bp, synthetic construct
  source 1..49: mol_type = other DNA
tagtaggtctcatagtcgatacagtcaactgtctttgacctttgttact

SEQ ID NO: 207 - DNA, 39 bp, synthetic construct
  source 1..39: mol_type = other DNA
tagtaggtctcactcaatggatgctgatgagggtcaaga

SEQ ID NO: 208 - DNA, 53 bp, synthetic construct
  source 1..53: mol_type = other DNA
tagtaggtctcatcattgagcctgagccgctcatgtggaagcggtgctcccct

SEQ ID NO: 209 - DNA, 57 bp, synthetic construct
  source 1..57: mol_type = other DNA
tagtaggtctcactcaatggaagatatacaaacaaatgcggaactgaaaagcactca

SEQ ID NO: 210 - DNA, 53 bp, synthetic construct
  source 1..53: mol_type = other DNA
tagtaggtctcatcattgagcctgagcccttcagcagggctctgtgttctcct

SEQ ID NO: 211 - DNA, 59 bp, synthetic construct
  source 1..59: mol_type = other DNA
aggtctcaggtggcggctcaatggccggcgaaggagatcagcaggacgctgcgcacaac

SEQ ID NO: 212 - DNA, 49 bp, synthetic construct
  source 1..49: mol_type = other DNA
tagtggtctcatagtcaagcaaagtattactttgtctggacttatttca

SEQ ID NO: 213 - DNA, 55 bp, synthetic construct
  source 1..55: mol_type = other DNA
tagtaggtctcactcaatggccggcgaaggagatcagcaggacgctgcgcacaac

SEQ ID NO: 214 - DNA, 49 bp, synthetic construct
  source 1..49: mol_type = other DNA
tagtggtctcacacccaagcaaagtattactttgtctggacttatttca

SEQ ID NO: 215 - DNA, 45 bp, synthetic construct; identical to SEQ ID NO: 109
  source 1..45: mol_type = other DNA

SEQ ID NO: 216 - DNA, 47 bp, synthetic construct; identical to SEQ ID NO: 110
  source 1..47: mol_type = other DNA

SEQ ID NO: 217 - DNA, 38 bp, synthetic construct
  source 1..38: mol_type = other DNA
tatagtggtctcactcaatgggagtgcaggtggaaacc

SEQ ID NO: 218 - DNA, 47 bp, synthetic construct
  source 1..47: mol_type = other DNA
tagtaggtctcacaccttccagttttagaagctccacatcgaagacg

SEQ ID NO: 219 - DNA, 54 bp, synthetic construct
  source 1..54: mol_type = other DNA
tagtaggtctcaggtggcggctcaatgaagctactgtcttctatcgaacaagca

SEQ ID NO: 220 - DNA, 49 bp, synthetic construct; identical to SEQ ID NO: 206
  source 1..49: mol_type = other DNA

SEQ ID NO: 221 - DNA, 45 bp, synthetic construct; identical to SEQ ID NO: 95
  source 1..45: mol_type = other DNA

SEQ ID NO: 222 - DNA, 49 bp, synthetic construct; identical to SEQ ID NO: 96
  source 1..49: mol_type = other DNA

SEQ ID NO: 223 - DNA, 39 bp, synthetic construct
  source 1..39: mol_type = other DNA
tagtaggtctcactcaagagctgaccccaagaagaagag

SEQ ID NO: 224 - DNA, 59 bp, synthetic construct
  source 1..59: mol_type = other DNA
tagggtctcacaccaaacaaacttgtgtcaaatatactcagcccggtgctgatgtgcat

SEQ ID NO: 225 - DNA, 47 bp, synthetic construct; identical to SEQ ID NO: 73
  source 1..47: mol_type = other DNA

SEQ ID NO: 226 - DNA, 59 bp, synthetic construct; identical to SEQ ID NO: 74
  source 1..59: mol_type = other DNA

SEQ ID NO: 227 - DNA, 54 bp, synthetic construct; identical to SEQ ID NO: 219
  source 1..54: mol_type = other DNA

SEQ ID NO: 228 - DNA, 49 bp, synthetic construct; identical to SEQ ID NO: 206
  source 1..49: mol_type = other DNA

SEQ ID NO: 229 - DNA, 42 bp, synthetic construct
  source 1..42: mol_type = other DNA
tagtaggtctcactcagagagcacacctattcagcaactcct

SEQ ID NO: 230 - DNA, 48 bp, synthetic construct
  source 1..48: mol_type = other DNA
tagtaggtctcacaccgctcatcatcttaaagcctgcgtgaaggatct

SEQ ID NO: 231 - DNA, 59 bp, synthetic construct
  source 1..59: mol_type = other DNA
tagtggtctcactcaatgagtcggctggataaatctaaagtcataaactctgctctgga

SEQ ID NO: 232 - DNA, 36 bp, synthetic construct
  source 1..36: mol_type = other DNA
tagtaggtctcacggtggtaggtgtctctctttcct

SEQ ID NO: 233 - DNA, 45 bp, synthetic construct
  source 1..45: mol_type = other DNA
tagtaggtctcaaccgattctatgccaccacttctgagacaagca

SEQ ID NO: 234 - DNA, 53 bp, synthetic construct
  source 1..53: mol_type = other DNA
tagtggtctcacgatactgagccgccaccggccggcccgccgctttcgcactt

SEQ ID NO: 235 - DNA, 45 bp, synthetic construct; identical to SEQ ID NO: 109
  source 1..45: mol_type = other DNA

SEQ ID NO: 236 - DNA, 47 bp, synthetic construct; identical to SEQ ID NO: 110
  source 1..47: mol_type = other DNA

SEQ ID NO: 237 - DNA, 59 bp, synthetic construct; identical to SEQ ID NO: 231
  source 1..59: mol_type = other DNA

SEQ ID NO: 238 - DNA, 36 bp, synthetic construct; identical to SEQ ID NO: 232
  source 1..36: mol_type = other DNA

SEQ ID NO: 239 - DNA, 45 bp, synthetic construct; identical to SEQ ID NO: 233
  source 1..45: mol_type = other DNA

SEQ ID NO: 240 - DNA, 53 bp, synthetic construct; identical to SEQ ID NO: 234
  source 1..53: mol_type = other DNA

SEQ ID NO: 241 - DNA, 50 bp, synthetic construct
  source 1..50: mol_type = other DNA
tagtaggtctcaggtggcggctcagagagcacacctattcagcaactcct

SEQ ID NO: 242 - DNA, 50 bp, synthetic construct; identical to SEQ ID NO: 126
  source 1..50: mol_type = other DNA

SEQ ID NO: 243 - DNA, 46 bp, synthetic construct
  source 1..46: mol_type = other DNA
gatatggtctcactcactaaaagatgattctcttccttcaaatcca

SEQ ID NO: 244 - DNA, 59 bp, synthetic construct; identical to SEQ ID NO: 74
  source 1..59: mol_type = other DNA

SEQ ID NO: 245 - DNA, 51 bp, synthetic construct
  source 1..51: mol_type = other DNA
tagtaggtctcaaatggacagaatcaagaaacagctacgtgaatgggatga

SEQ ID NO: 246 - DNA, 59 bp, synthetic construct; identical to SEQ ID NO: 74
  source 1..59: mol_type = other DNA

SEQ ID NO: 247 - DNA, 47 bp, synthetic construct
  source 1..47: mol_type = other DNA
tagtaggtctcacattaaggtttcagcaggcaacacacattcgggaa

SEQ ID NO: 248 - DNA, 55 bp, synthetic construct; identical to SEQ ID NO: 213
  source 1..55: mol_type = other DNA

SEQ ID NO: 249 - DNA, 1171 bp, synthetic construct
  source 1..1171: mol_type = other DNA
  misc_feature 4..24: note = SV40 NLS
  misc_feature 55..1024: note = Micro-deleted Cre
  misc_feature 1025..1099: note = SV40 NLS
  misc_feature 1118..1165: note = Nucleoplasmin NLS
atgccaaagaagaagcggaaggtcggtggcggctcacccgggggtggcggctcaatgaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatccaaagaagaagcggaaagtcactagtggtggcggctcaaagcgtcctgctgctactaagaaagctggtcaagctaagaaaaagaaatctaga

SEQ ID NO: 250 - DNA, 34 bp, synthetic construct
  source 1..34: mol_type = other DNA; note = LoxP site
ataacttcgtatagcatacattatacgaagttat

SEQ ID NO: 251 - DNA, 32 bp, synthetic construct
  source 1..32: mol_type = other DNA; note = Rox site
taactttaaataattggcattatttaaagtta

SEQ ID NO: 252 - DNA, 238 bp, synthetic construct
  source 1..238: mol_type = other DNA; note = pUAS-1
  misc_feature 1..93: note = GAL4 DBD binding sites
  misc_feature 111..238: note = 3G miniPromoter
cggagtactgtcctccgagcggagtactgtcctccgagcggagtactgtcctccgagcggagtactgtcctccgagcggagttctgtcctccgagcggagactctagactggcgtgtacggtgggcgcctataaaagcagagctcgtttagtgaaccgtcagatcgcctggagcaattccacaacacttttgtcttataccaactttccgtaccacttcctaccctcgtaaagtgacc

SEQ ID NO: 253 - DNA, 281 bp, synthetic construct
  source 1..281: mol_type = other DNA; note = pUAS-2
  misc_feature 23..39: note = GAL4 DBD binding site
  misc_feature 42..58: note = GAL4 DBD binding site
  misc_feature 66..82: note = GAL4 DBD binding site
  misc_feature 85..101: note = GAL4 DBD binding site
  misc_feature 109..125: note = GAL4 DBD binding site
  misc_feature 137..153: note = GAL4 DBD binding site
  misc_feature 156..172: note = GAL4 DBD binding site
  misc_feature 180..196: note = GAL4 DBD binding site
  misc_feature 203..243: note = LateADE miniPromoter
cgataggtaccgagtttctagacggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccggctagcggggggctataaaagggggtgggggcgttcgtcctcactctagatctgcgatctaagtaagcttggcattccggtactg

SEQ ID NO: 254 - DNA, 20 bp, synthetic construct
  source 1..20: mol_type = other DNA
gttacgaaaacctaggggtg

SEQ ID NO: 255 - DNA, 22 bp, synthetic construct
  source 1..22: mol_type = other DNA
tgaaagtggcatcttgaaaggg

SEQ ID NO: 256 - DNA, 21 bp, synthetic construct
  source 1..21: mol_type = other DNA
acccttgcattccaataccac

SEQ ID NO: 257 - DNA, 20 bp, synthetic construct
  source 1..20: mol_type = other DNA
agatccagggacacggtgct

SEQ ID NO: 258 - DNA, 18 bp, synthetic construct
  source 1..18: mol_type = other DNA
gtgggaaacagccgtcag

SEQ ID NO: 259 - DNA, 18 bp, synthetic construct
  source 1..18: mol_type = other DNA
cactgagcactgaaggcc

SEQ ID NO: 260 - DNA, 20 bp, synthetic construct
  source 1..20: mol_type = other DNA
aaaaccgaggggtaagaatc

SEQ ID NO: 261 - DNA, 56 bp, synthetic construct
  source 1..56: mol_type = other DNA
acactctttccctacacgacgctcttccgatctataggataggagtgatggacagg

SEQ ID NO: 262 - DNA, 52 bp, synthetic construct
  source 1..52: mol_type = other DNA
gactggagttcagacgtgtgctcttccgatctctgctgctcctcaatacacc

SEQ ID NO: 263 - DNA, 20 bp, synthetic construct
  source 1..20: mol_type = other DNA
gacaaaccagaagccgctcc

SEQ ID NO: 264 - DNA, 25 bp, synthetic construct
  source 1..25: mol_type = other DNA
tctcttgtggtttcctagcttctga

SEQ ID NO: 265 - DNA, 24 bp, synthetic construct
  source 1..24: mol_type = other DNA
actttcccctgagtttaagtgatg

SEQ ID NO: 266 - DNA, 20 bp, synthetic construct
  source 1..20: mol_type = other DNA
gaacacaaagcatagactgc

SEQ ID NO: 267 - DNA, 24 bp, synthetic construct
  source 1..24: mol_type = other DNA
acatttgggcttctttctagttga

SEQ ID NO: 268 - DNA, 25 bp, synthetic construct
  source 1..25: mol_type = other DNA
cctgatgtaatgactagactgaggc

SEQ ID NO: 269 - DNA, 20 bp, synthetic construct
  source 1..20: mol_type = other DNA
ggcccagactgagcacgtga

SEQ ID NO: 270 - DNA, 65 bp, synthetic construct
  source 1..65: mol_type = other DNA
tggaggaagcagggcttcctttcctctgccatcaatgatggtgatgatggtgcgtgctcagtctg

SEQ ID NO: 271 - DNA, 20 bp, synthetic construct
  source 1..20: mol_type = other DNA
aaatgccagattacgtatcc

SEQ ID NO: 272 - DNA, 27 bp, synthetic construct
  source 1..27: mol_type = other DNA
tcgctgccaggatatacgtaatctggc

SEQ ID NO: 273 - DNA, 20 bp, synthetic construct
  source 1..20: mol_type = other DNA
gtcaaccagtatcccggtgc

SEQ ID NO: 274 - DNA, 20 bp, synthetic construct
  source 1..20: mol_type = other DNA
cgaacgcactgatttcgacc

SEQ ID NO: 275 - DNA, 20 bp, synthetic construct
  source 1..20: mol_type = other DNA
cttttcctctgttgagctcg

SEQ ID NO: 276 - DNA, 20 bp, synthetic construct
  source 1..20: mol_type = other DNA
gaatcagtgctggagaatgg

SEQ ID NO: 277 - DNA, 23 bp, synthetic construct
  source 1..23: mol_type = other DNA
cgtgctacttcatgtggtcgggg

SEQ ID NO: 278 - DNA, 23 bp, synthetic construct
  source 1..23: mol_type = other DNA
cttctacttgtcggccatgatgt